(19) United States
(12) Patent Application Publication
(10) Pub. No.: US 2008/0096829 A1
(43) Pub. Date: Apr. 24, 2008

(54) Macrolone Compounds

(76) Inventors: Sulejman Alihodzic, Zagreb (HR); Catherine Simone Victoire Frydrych, Essex (GB); Erie Hunt, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/718,732

(22) PCT Filed: Nov. 9, 2005
(86) PCT No.: PCT/EP05/12039

§ 371 Date: May 7, 2007

(30) Foreign Application Priority Data
Nov. 11, 2004 (GB) 0424961.1

(51) Int. Cl. A61K 31/7048; A61P 31/00; C07H 17/08

(52) U.S. Cl. 514/29; 536/7.1

(57) ABSTRACT

A compound of formula (I) 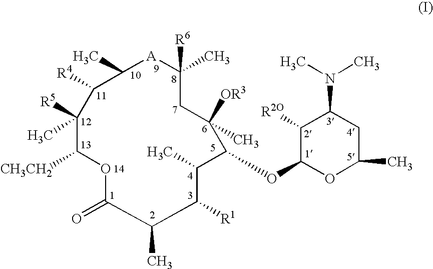 For use as antibacterial agents, compositions containing same, processes for their preparation and their use in therapy.

The present invention relates to novel semi-synthetic macrolides having antimicrobial activity, in particular antibacterial activity. More particularly, the invention relates to 14- and 15-membered macrolides substituted at the 3 position, to processes for their preparation, to compositions containing them and to their use in medicine.

Macrolide antibacterial agents are known to be useful in the treatment or prevention of bacterial infections. However, the emergence of macrolide-resistant bacterial strains has resulted in the need to develop new macrolide compounds.

According to the present invention, we have now found novel 14- and 15-membered macrolides substituted at the 3 position which also have antimicrobial activity.

Thus, the present invention provides compounds of general formula (I) 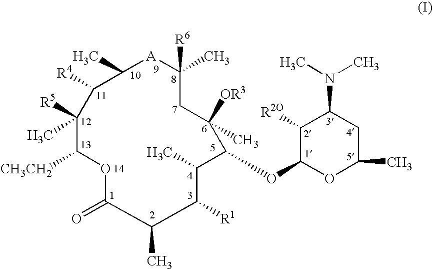
wherein
A is a bivalent radical —C(O)—, —N(R⁷)—CH₂—, —CH(NR⁸R⁹)— or —C(═NR¹⁰)—, or A and R⁴ taken together with the intervening atoms form a cyclic group having the following formula: 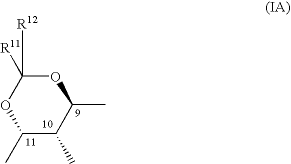
and R¹ is a group having the following formula: 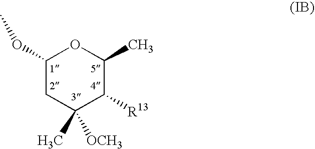
wherein R¹³ is —OC(O)(CH₂)_{d}U¹R¹⁴, —OC(O)N(R¹⁵)(CH₂)_{d}U¹R¹⁴, —O(CH₂)_{d}U¹R¹⁴, 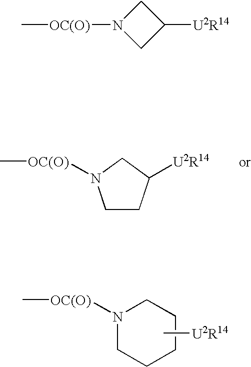
or
A is the bivalent radical —N(R⁷)—CH₂— and R¹ is a group having the following formula: 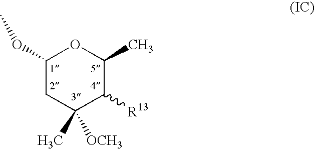
wherein R¹³ is —NHC(O)(CH₂)_{d}U¹R¹⁴;
R² is hydrogen or a hydroxyl protecting group;
R³ is hydrogen, C₁₋₄alkyl, or C₃₋₆alkenyl optionally substituted by 9- or 10-membered fused bicyclic heteroaryl;
R⁴ is hydroxy, C₃₋₆alkenyloxy optionally substituted by 9- or 10-membered fused bicyclic heteroaryl, or C₁₋₆alkoxy optionally substituted by C₁₋₆alkoxy or —O(CH₂)ₑNR⁷R¹⁶, or R⁴ and A taken together with the intervening atoms form a cyclic group of formula (IA),
R⁵ is hydroxy, or
R⁴ and R⁵ taken together with the intervening atoms form a cyclic group having the following formula: 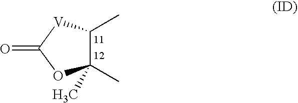
wherein V is a bivalent radical —CH₂—, —CH(CN)—, —O—, —N(R¹⁷)— or —CH(SR¹⁷)—, with the proviso that when R¹ is a group of formula (IC), V is —O—;
R⁶ is hydrogen or fluorine;
R⁷ is hydrogen or C₁₋₆alkyl;
R⁸ and R⁹ are each independently hydrogen, C₁₋₆alkyl or —C(O)R¹⁸, or
R⁸ and R⁹ together form ═CH(CR¹⁸R¹⁹)_{f}aryl, ═CH(CR¹⁸R¹⁹)_{f}heterocyclyl, ═CR¹⁸R¹⁹ or ═C(R¹⁸)C(O)OR⁸, wherein the alkyl, aryl and heterocyclyl groups are optionally substituted by up to three groups independently selected from R²⁰;
R¹⁰ is —OR²¹;
R¹¹ and R¹² are each independently hydrogen, C₁₋₆alkyl, heteroaryl, or aryl optionally substituted by one or two groups independently selected from hydroxyl and C₁₋₆alkoxy;
R¹⁴ is a heterocyclic group having the following formula: 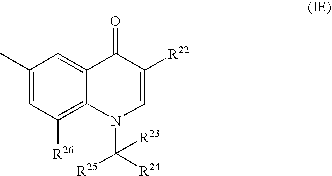
R¹⁵, R¹⁶, R¹⁸ and R¹⁹ are each independently hydrogen or C₁₋₆alkyl;
R¹⁷ is hydrogen or C₁₋₄alkyl optionally substituted by a group selected from optionally substituted phenyl, optionally substituted 5- or 6-membered heteroaryl and optionally substituted 9- or 10-membered fused bicyclic heteroaryl;
R²⁰ is halogen, cyano, nitro, trifluoromethyl, azido, —C(O)R²⁷, —C(O)OR²⁷, —OC(O)R²⁷, —OC(O)OR²⁷, —NR²⁸C(O)R²⁹, —C(O)NR²⁸R²⁹, —NR²⁸R²⁹, hydroxy, C₁₋₆alkyl, —S(O)_{g}C₁₋₆alkyl, C₁₋₆alkoxy, —(CH₂)ₕaryl or —(CH₂)ₕheteroaryl, wherein the alkoxy group is optionally substituted by up to three groups independently selected from —NR¹⁸R¹⁹, halogen and —OR¹⁸, and the aryl and heteroaryl groups are optionally substituted by up to five groups independently selected from halogen, cyano, nitro, trifluoromethyl, azido, —C(O)R³⁰, —C(O)OR³⁰, —OC(O)OR³⁰, —NR³¹C(O)R³², —C(O)NR³¹R³², —NR³¹R³², hydroxy, C₁₋₆alkyl and C₁₋₆alkoxy;
R²¹ is hydrogen, C₁₋₆alkyl, C₃₋₇cycloalkyl, C₃₋₆alkenyl or a 5- or 6-membered heterocyclic group, wherein the alkyl, cycloalkyl, alkenyl and heterocyclic groups are optionally substituted by up to three groups independently selected from optionally substituted 5- or 6-membered heterocyclic group, optionally substituted 5- or 6-membered heteroaryl, —OR³³, —S(O)ᵢR³³, —NR³³R³⁴, —CONR³³R³⁴, halogen and cyano;
R²² is —C(O)OR³⁵, —C(O)NHR³⁵, —C(O)CH₂NO₂ or —C(O)CH₂SO₂R⁷;
R²³ and R²⁴ are each independently hydrogen or methyl;
R²⁵ and R²⁶ are linked to form a bivalent radical —OCH₂—, —CH₂O—, —O(CH₂)₂—, —CH₂OCH₂— or —(CH₂)₂O—;
R²⁷ is hydrogen, C₁₋₁₀alkyl, —(CH₂)ⱼaryl or —(CH₂)ⱼheteroaryl;
R²⁸ and R²⁹ are each independently hydrogen, —OR¹⁸, C₁₋₆alkyl, —(CH₂)ₖaryl or —(CH₂)ₖheterocyclyl;
R³⁰ is hydrogen, C₁₋₁₀alkyl, —(CH₂)ₘaryl or —(CH₂)ₘheteroaryl;
R³¹ and R³² are each independently hydrogen, —OR¹⁸, C₁₋₆alkyl, —(CH₂)ₙaryl or —(CH₂)ₙheterocyclyl;
R³³ and R³⁴ are each independently hydrogen, C₁₋₄alkyl or C₁₋₄alkoxyC₁₋₄alkyl;
R³⁵ is hydrogen,
C₁₋₆alkyl optionally substituted by up to three groups independently selected from halogen, cyano, C₁₋₄alkoxy optionally substituted by phenyl or C₁₋₄alkoxy, —C(O)C₁₋₆alkyl, —C(O)OC₁₋₆alkyl, —OC(O)C₁₋₆alkyl, —OC(O)OC₁₋₆alkyl, —C(O)NR³⁶R³⁷,
—NR³⁶R³⁷ and phenyl optionally substituted by nitro or —C(O)OC₁₋₆alkyl,
—(CH₂)ₚC₃₋₇cycloalkyl,
—(CH₂)ₚheterocyclyl,
—(CH₂)ₚheteroaryl,
—(CH₂)ₚaryl,
C₃₋₆alkenyl, or
C₃₋₆alkynyl;
   R³⁶ and R³⁷ are each independently hydrogen or C₁₋₆alkyl optionally substituted by phenyl or —C(O)OC₁₋₆alkyl, or
   R³⁶ and R³⁷, together with the nitrogen atom to which they are bound, form a 5- or 6-membered heterocyclic group optionally containing one additional heteroatom selected from oxygen, sulfur and N—R³⁸;
   R³⁸ is hydrogen or methyl;
   R³⁹ is hydrogen, C₁₋₄alkyl, C₃₋₇cycloalkyl, optionally substituted phenyl or benzyl, acetyl or benzoyl;
   U¹ is a bivalent radical —W(CH₂)_{q}X—, —W(CH₂)_{q}—, —W(CH₂)_{q}X(CH₂)ᵣY—, —W(CH₂)_{q}X(CH₂)ᵣ—, —W(CH₂)_{q}X(CH₂)ᵣY(CH₂)ₛZ- or —W(CH₂)_{q}X(CH₂)ᵣY(CH₂)ₛ—;
   U² is U¹ or a bivalent radical —O—, —N(R³⁹)—, —S(O)ₜ— or —CH₂—;
   W, X, Y and Z are each independently a bivalent radical —N(R³⁹)—, —O—, —S(O)ₜ—, —N(R³⁹)C(O)—, —C(O)N(R³⁹)— or —N[C(O)R³⁹]—;
   d is an integer from 2 to 5;
   e is an integer from 2 to 4;
   f, h, j, k, m, n and p are each independently integers from 0 to 4;
   g, i and t are each independently integers from 0 to 2; and
   q, r and s are each independently integers from 2 to 5;
   with the proviso that when R²³ and R²⁴ are each hydrogen, R²⁵ and R²⁶ are not linked to form the bivalent radical —CH₂O—, and
   when R²³ and R²⁴ are each hydrogen and U¹ is —W(CH₂)_{q}X(CH₂)ᵣY—, —W(CH₂)_{q}X(CH₂)ᵣ—, —W(CH₂)_{q}X(CH₂)ᵣY(CH₂)ₛZ- or —W(CH₂)_{q}X(CH₂)ᵣY(CH₂)ₛ, R²⁵ and R²⁶ are not linked to form the bivalent radical —OCH₂—;
   and pharmaceutically acceptable derivatives thereof.

The term “pharmaceutically acceptable” as used herein means a compound which is suitable for pharmaceutical use. Salts and solvates of compounds of the invention which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of the invention and their pharmaceutically acceptable salts and solvates. The term “pharmaceutically acceptable derivative” as used herein means any pharmaceutically acceptable salt, solvate or prodrug, eg ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives. Examples of pharmaceutically acceptable derivatives are salts, solvates, esters, carbamates and phosphate esters. Additional examples of pharmaceutically acceptable derivatives are salts, solvates and esters. Further examples of pharmaceutically acceptable derivatives are salts and esters, such as salts.

The compounds of the present invention may be in the form of and/or may be administered as a pharmaceutically acceptable salt. For a review on suitable salts see Berge et al., J. Pharm. Sci., 1977, 66, 1-19.

Typically, a pharmaceutical acceptable salt may be readily prepared by using a desired acid or base as appropriate. The salt may precipitate from solution and be collected by filtration or may be recovered by evaporation of the solvent. For example, an aqueous solution of an acid such as lactobionic acid may be added to a solution of a compound of formula (I) in a solvent such as acetonitrile, acetone or THF, and the resulting mixture evaporated to dryness, redissolved in water and lyophilised to obtain the acid addition salt as a solid. Alternatively, a compound of formula (I) may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The skilled person will appreciate that where the compound of formula (I) contains more than one basic group bis salts (2:1 acid:compound of formula (I)) or tris salts (3:1 acid:compound of formula (I)) may also be formed and are salts according to the present invention.

Suitable addition salts are formed from inorganic or organic acids which form non-toxic salts and examples are lactobionate, mandelate (including (S)-(+)-mandelate, (R)-(−)-mandelate and (R,S)-mandelate), hydrochloride, hydrobromide, hydroiodide, sulfate, bisulfate, nitrate, phosphate, hydrogen phosphate, acetate, trifluoroacetate, maleate, malate, fumarate, lactate, tartrate, citrate, formate, gluconate, succinate, ethyl succinate (4-ethoxy-4-oxo-butanoate), pyruvate, oxalate, oxaloacetate, saccharate, benzoate, alkyl or aryl sulphonates (eg methanesulphonate, ethanesulphonate, benzenesulphonate or p-toluenesulphonate) and isethionate. In one embodiment, suitable salts include lactobionate, citrate, succinate, (L)-(+)-tartrate, (S)-(+)-mandalete and bis-(S)-(+)-mandalete, for example lactobionate, citrate, succinate and (L)-(+)-tartrate, such as lactobionate and citrate.

Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

Compounds of the invention may have both a basic and an acidic centre may therefore be in the form of zwitterions.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compounds of the invention are within the scope of the invention. The salts of the compound of formula (I) may form solvates (eg hydrates) and the invention also includes all such solvates.

The term “prodrug” as used herein means a compound which is converted within the body, eg by hydrolysis in the blood, into its active form that has medical effects. Pharmaceutically acceptable prodrugs are described in T. Higuchi and V. Stella, “Prodrugs as Novel Delivery Systems”, Vol. 14 of the A.C.S. Symposium Series; Edward B. Roche, ed., “Bioreversible Carriers in Drug Design”, American Pharmaceutical Association and Pergamon Press, 1987; and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of formula (I) in vivo, when the prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Representative examples of prodrugs include (but are not limited to) acetate, formate and benzoate derivatives of alcohol, sulfhydryl and amine functional groups of the compounds of formula (I). Further, in the case of a carboxylic acid (—COOH), esters may be employed, such as methyl esters, ethyl esters, and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

References hereinafter to a compound according to the invention include both compounds of formula (I) and their pharmaceutically acceptable derivatives.

With regard to stereoisomers, the compounds of formula (I) have more than one asymmetric carbon atom. In the general formula (I) as drawn, the solid wedge shaped bond indicates that the bond is above the plane of the paper. The broken bond indicates that the bond is below the plane of the paper. The wavy bond ( [symbol] ) indicates that the bond can be either above or below the plane of the paper. Thus, when R′ is a group of formula (IC), the present invention includes both epimers at the 4″ carbon, and mixtures thereof.

It will be appreciated that the substituents on the macrolide may also have one or more asymmetric carbon atoms. Thus, the compounds of structure (I) may occur as individual enantiomers or diastereomers. All such isomeric forms are included within the present invention, including mixtures thereof.

Where a compound of the invention contains an alkenyl group, cis (Z) and trans (E) isomerism may also occur. The present invention includes the individual stereoisomers of the compound of the invention and, where appropriate, the individual tautomeric forms thereof, together with mixtures thereof.

Separation of diastereoisomers or cis and trans isomers may be achieved by conventional techniques, eg by fractional crystallisation, chromatography or HPLC. A stereoisomeric mixture of the agent may also be prepared from a corresponding optically pure intermediate or by resolution, such as by HPLC, of the corresponding mixture using a suitable chiral support or by fractional crystallisation of the diastereoisomeric salts formed by reaction of the corresponding mixture with a suitable optically active acid or base, as appropriate.

The compounds of formula (I) may be in crystalline or amorphous form. Furthermore, some of the crystalline forms of the compounds of structure (I) may exist as polymorphs, which are included in the present invention.

Compounds wherein R² represents a hydroxyl protecting group are in general intermediates for the preparation of other compounds of formula (I).

When the group OR² is a protected hydroxyl group this is conveniently an ether or an acyloxy group. Examples of particularly suitable ether groups include those in which R² is a trialkylsilyl (i.e. trimethylsilyl). When the group OR² represents an acyloxy group, then examples of suitable groups R² include acetyl or benzoyl.

When R¹³ is 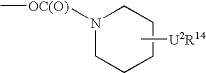
the —U²R¹⁴ group is typically attached at the 3- or 4-position on the piperidine ring.

The term “alkyl” as used herein as a group or a part of a group refers to a straight or branched hydrocarbon chain containing the specified number of carbon atoms. For example, C₁₋₁₀alkyl means a straight or branched alkyl containing at least 1, and at most 10, carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl. A C₁₋₄alkyl group is preferred, for example methyl, ethyl, n-propyl, isopropyl, n-butyl, isobutyl or t-butyl.

The term “C₃₋₇cycloalkyl” group as used herein refers to a non-aromatic monocyclic hydrocarbon ring of 3 to 7 carbon atoms such as, for example, cyclopropyl, cyclobutyl, cyclopentyl, cyclohexyl or cycloheptyl.

The term “alkoxy” as used herein refers to a straight or branched chain alkoxy group containing the specified number of carbon atoms. For example, C₁₋₆alkoxy means a straight or branched alkoxy containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy and hexyloxy. A C₁₋₄alkoxy group is preferred, for example methoxy, ethoxy, propoxy, prop-2-oxy, butoxy, but-2-oxy or 2-methylprop-2-oxy.

The term “alkenyl” as used herein as a group or a part of a group refers to a straight or branched hydrocarbon chain containing the specified number of carbon atoms and containing at least one double bond. For example, the term “C₂₋₆alkenyl” means a straight or branched alkenyl containing at least 2, and at most 6, carbon atoms and containing at least one double bond. Similarly, the term “C₃₋₆alkenyl” means a straight or branched alkenyl containing at least 3, and at most 6, carbon atoms and containing at least one double bond. Examples of “alkenyl” as used herein include, but are not limited to, ethenyl, 2-propenyl, 3-butenyl, 2-butenyl, 2-pentenyl, 3-pentenyl, 3-methyl-2-butenyl, 3-methylbut-2-enyl, 3-hexenyl and 1,1-dimethylbut-2-enyl. It will be appreciated that in groups of the form —O—C₂₋₆alkenyl, the double bond is preferably not adjacent to the oxygen.

The term “alkynyl” as used herein as a group or a part of a group refers to a straight or branched hydrocarbon chain containing the specified number of carbon atoms and containing at least one triple bond. For example, the term “C₃₋₆alkynyl” means a straight or branched alkynyl containing at least 3, and at most 6, carbon atoms and containing at least one triple bond. Examples of “alkynyl” as used herein include, but are not limited to, propynyl, 1-butynyl, 2-butynyl, 1-pentynyl and 3-methyl-1-butynyl.

The term “aryl” as used herein refers to an aromatic carbocyclic moiety such as phenyl, biphenyl or naphthyl, for example phenyl.

The term “heteroaryl” as used herein, unless otherwise defined, refers to an aromatic heterocycle of 5 to 10 members, having at least one heteroatom selected from nitrogen, oxygen and sulfur, and containing at least 1 carbon atom, including both mono and bicyclic ring systems. Examples of heteroaryl rings include, but are not limited to, furanyl, thiophenyl, pyrrolyl, pyrazolyl, imidazolyl, oxazolyl, isoxazolyl, thiazolyl, isothiazolyl, triazolyl, oxadiazolyl, tetrazolyl, thiadiazolyl, pyridyl, pyridazinyl, pyrazinyl, pyrimidinyl, triazinyl, quinolinyl, isoquinolinyl, 1,2,3,4-tetrahydroisoquinolinyl, benzofuranyl, benzimidazolyl, benzothienyl, benzoxazolyl, 1,3-benzodioxazolyl, indolyl, benzothiazolyl, furylpyridine, oxazolopyridyl and benzothiophenyl.

The term “5- or 6-membered heteroaryl” as used herein as a group or a part of a group refers to a monocyclic 5- or 6-membered aromatic heterocycle containing at least one heteroatom independently selected from oxygen, nitrogen and sulfur. Examples include, but are not limited to, furanyl, thiophenyl, pyrrolyl, pyrazolyl, imidazolyl, oxazolyl, isoxazolyl, thiazolyl, isothiazolyl, triazolyl, oxadiazolyl, tetrazolyl, pyridyl, pyridazinyl, pyrazinyl, pyrimidinyl and triazinyl.

The term “9- or 10-membered fused bicyclic heteroaryl” as used herein as a group or a part of a group refers to a 9- or 10-membered fused bicyclic heteroaryl containing at least one heteroatom selected from oxygen, nitrogen and sulphur. Examples include, but are not limited to, quinolinyl, isoquinolinyl, 1,2,3,4-tetrahydroisoquinolinyl, benzofuranyl, benzimidazolyl, benzothienyl, benzoxazolyl, 1,3-benzodioxazolyl, indolyl, benzothiazolyl, furylpyridine, oxazolopyridyl and benzothiophenyl.

The term “heterocyclyl” as used herein, unless otherwise defined, refers to a monocyclic or bicyclic 3- to 10-membered saturated or non-aromatic, unsaturated hydrocarbon ring containing at least one heteroatom selected from oxygen, nitrogen and sulfur. Preferably, the heterocyclyl ring has five or six ring atoms. Examples of heterocyclyl groups include, but are not limited to, pyrrolidinyl, tetrahydrofuranyl, tetrahydrothiophenyl, imidazolidinyl, pyrazolidinyl, piperidyl, piperazinyl, morpholino, tetrahydropyranyl and thiomorpholino.

The term “5- or 6-membered heterocyclic group” as used herein as a group or part of a group refers to a monocyclic 5- or 6-membered saturated hydrocarbon ring containing at least one heteroatom independently selected from oxygen, nitrogen and sulfur. Examples of such heterocyclyl groups include, but are not limited to, pyrrolidinyl, tetrahydrofuranyl, tetrahydrothiophenyl, imidazolidinyl, pyrazolidinyl, piperidyl, piperazinyl, morpholino, tetrahydropyranyl and thiomorpholino.

The term “halogen” refers to a fluorine, chlorine, bromine or iodine atom, such as fluorine or chloride.

The terms “optionally substituted phenyl”, “optionally substituted phenyl or benzyl”, “optionally substituted 5- or 6-membered heteroaryl”, “optionally substituted 9- or 10-membered fused bicyclic heteroaryl” or “optionally substituted 5- or 6-membered heterocyclic group” as used herein refer to a group which is substituted by 1 to 3 groups independently selected from halogen, C₁₋₄alkyl, C₁₋₄alkoxy, hydroxy, nitro, cyano, amino, C₁₋₄alkylamino or diC₁₋₄alkylamino, phenyl and 5- or 6-membered heteroaryl.

As the skilled person will appreciate, the compounds of formula (I) are derivatives of known 14- and 15-membered macrolides derived from erythromycin A that have antibacterial activity and contain a cladinose moiety with a hydroxy group or amino group at 4″ position. The 14- and 15-membered macrolides which may be derivatised according to the invention include, for example, the following:
azithromycin,
11-O-methyl-azithromycin,
azithromycin 11,12-carbonate,
6-O-methyl erythromycin A,
6-O-methyl erythromycin A 9-oxime,
6-O-methyl erythromycin A 9-O-alkyl-oximes,
erythromycin 9-oxime,
erythromycin 9-O-alkyl-oximes,
erythromycin 11,12-carbonate,
erythromycin 9-oxime 11,12-carbonate,
6-O-methyl-11-deoxy-11-amino-erythromycin A 11,12-carbamate,
(9S)-9-dihydro-erythromycin, and
(9S)-9-dihydro-erythromycin 9,11-ethylidene acetal,
   and the above macrolides in which the 4″ hydroxyl group is replaced by an amino group to give a macrolide having (R), (S) or (R,S) stereochemistry at the 4″ position.

In the compounds of formula (I), the heterocyclic group of formula (IE) (R¹⁴) is attached to the 4″ position of the 14- or 15-membered macrolide via a linker chain. Linker chains suitable for use according to the present invention include, for example, the following: —OC(O)(CH₂)₂NH(CH₂)₃—.

Representative examples of A include —C(O)—, —N(R⁷)—CH₂— and —C(═NR¹⁰)—, for example —C(O)— and —C(═NR¹⁰)—.

A representative example of R¹ is

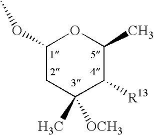

A representative example of R² is hydrogen.

Representative examples of R³ include hydrogen and C₁₋₄alkyl, such as hydrogen and methyl, for example hydrogen.

In one embodiment, R⁴ and R⁵ are hydroxy, or R⁴ and R⁵ taken together with the intervening atoms form a cyclic group having the following structure: 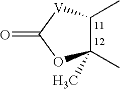
wherein V is a bivalent radical selected from —CH₂—, —CH(CN)—, —O—, —N(R¹⁷)— or —CH(SR¹⁷)—, such as —O—.

In a further embodiment, R⁴ and R⁵ are hydroxy.

A representative example of R⁶ is hydrogen.

A representative example of R⁷ is C₁₋₄alkyl, for example methyl.

In one embodiment, R¹¹ and R¹² are each independently hydrogen or C₁₋₆alkyl. In a further embodiment, one of R¹¹ and R¹² is hydrogen and the other is methyl.

A representative example of R¹³ is —OC(O)(CH₂)_{d}U¹R¹⁴.

Representative examples of R¹⁴ include heterocyclic groups having one of the following formulae:

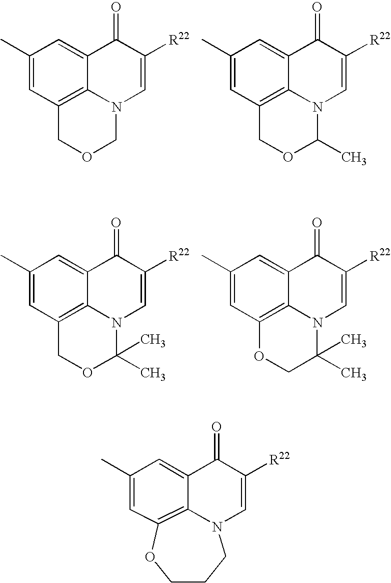

For example, R¹⁴ may be a heterocyclic group having one of the following formulae:

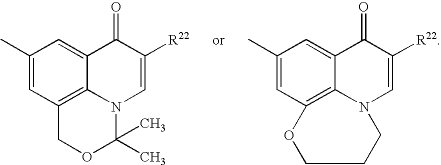

In one embodiment, R¹⁵ is hydrogen.

In one embodiment, R¹⁷ is hydrogen.

Representative examples of R²¹ include hydrogen and C₁₋₄alkyl optionally substituted by —OR³³, for example hydrogen and methyl optionally substituted by —OR³³.

In one embodiment, R²² is —C(O)OR³⁵, —C(O)NHR³⁵ or —C(O)CH₂NO₂. A representative example of R²² is —C(O)₂R³⁵.

In one embodiment, when one of R²³ and R²⁴ is hydrogen and the other is methyl, or R²³ and R²⁴ are each methyl, R²⁵ and R²⁶ are linked to form a bivalent radical —OCH₂—, —CH₂O—, —O(CH₂)₂—, —CH₂OCH₂— or —(CH₂)₂O—; when R²³ and R²⁴ are each hydrogen and U¹ is —W(CH₂)_{q}X— or W(CH₂)_{q}—, R²⁵ and R²⁵ are linked to form a bivalent radical —OCH₂—, —O(CH₂)₂—, —CH₂OCH₂— or —(CH₂)₂O—; or when R²³ and R²⁴ are each hydrogen and U¹ is —W(CH₂)_{q}X(CH₂)ᵣY—, —W(CH₂)_{q}X(CH₂)ᵣ—, —W(CH₂)_{q}X(CH₂)ᵣY(CH₂)ₛZ- or —W(CH₂)_{q}X(CH₂)ᵣY(CH₂)ₛ—, R²⁵ and R²⁶ are linked to form a bivalent radical —O(CH₂)₂—, —CH₂OCH₂— or —(CH₂)₂O.

In one embodiment, one of R²³ and R²⁴ is hydrogen and the other is methyl, or R²³ and R²⁴ are each methyl.

In one embodiment, R²⁵ and R²⁶ are linked to form a bivalent radical —OCH₂—, —CH₂O— or —(CH₂)₂O—, such as —CH₂O—. In a further embodiment, R²⁵ and R²⁶ are linked to form a bivalent radical —O(CH₂)₂—, —CH₂OCH₂— or —(CH₂)₂O—.

A representative example of R³³ is C₁₋₄alkyl, for example methyl.

A representative example of R³⁵ is hydrogen.

A representative example of R³⁹ is hydrogen or methyl, such as hydrogen.

In one embodiment, U¹ is —W(CH₂)_{q}X— or W(CH₂)_{q}—.

A representative example of U¹ is —W(CH₂)_{q}—.

A representative example of V is —O—.

A representative example of W is —N(R³⁵)—.

A representative examples of d is 2.

A representative example of q is 3.

It is to be understood that the present invention covers all combinations of the embodiments and representative examples described hereinabove. It is also to be understood that the present invention encompasses compounds of formula (I) in which a particular group or parameter, for example R⁷, R¹⁸, R¹⁹, R²⁰, R²⁷, R²⁸, R²⁹, R³⁰, R³¹, R³², R³³, R³⁴, R³⁶, R³⁷, R³⁸, R³⁹, g, h, i, j, k, m, n and t may occur more than once. In such compounds it will be appreciated that each group or parameter is independently selected from the values listed.

Compounds of the invention include:
4″-O-{[(2-{[3-(6-carboxy-3-(R,S)-methyl-7-oxo-1H,7H-[1,3]oxazino[5,4,3-ij]quinolin-9-yl)propyl]amino}propionyl]-6-O-methyl erythromycin A,
4″-O-{[(2-{[3-(6-carboxy-3-(R,S)-methyl-7-oxo-1H,7H-[1,3]oxazino[5,4,3-ij]quinolin-9-yl)propyl]amino}propionyl]-azithromycin,
4″-O-{[(2-{[3-(6-carboxy-3-(R,S)-methyl-7-oxo-1H,7H-[1,3]oxazino[5,4,3-ij]quinolin-9-yl)propyl]amino}propionyl]-azithromycin-11,12-carbonate,
4″-[(2-{[3-(6-carboxy-3,3-dimethyl-7-oxo-1H,7H-[1,3]oxazino[5,4,3-ij]quinolin-9-yl)propyl]amino}propionyl]-azithromycin,
4″-O-[(2-{[3-(6-carboxy-3,3-dimethyl-7-oxo-1H,7H-[1,3]oxazino[5,4,3-ij]quinolin-9-yl)propyl]amino}propionyl]-6-O-methyl erythromycin A,
4″-O-[(2-{[3-(6-carboxy-3,3-dimethyl-7-oxo-1H,7H-[1,3]oxazino[5,4,3-ij]quinolin-9-yl)propyl]amino}propionyl]-azithromycin-11,12-carbonate,
4″-O-[(2-{[3-(6-carboxy-7-oxo-1H,7H-[1,3]oxazino[5,4,3-ij]quinolin-9-yl)propyl]amino}propionyl]-6-O-methyl erythromycin A,
4″-O-[(2-{[3-(6-carboxy-7-oxo-1H,7H-[1,3]oxazino[5,4,3-ij]quinolin-9-yl)propyl]amino}propionyl]-azithromycin,
4″-O-[(2-{[3-(6-carboxy-7-oxo-1H,7H-[1,3]oxazino[5,4,3-ij]quinolin-9-yl)propyl]amino}propionyl]-azithromycin-11,12-carbonate,
4″-O-[(2-{[3-(6-carboxy-3,3-dimethyl-7-oxo-1H,7H-[1,3]oxazino[5,4,3-ij]quinolin-9-yl)propyl]amino}propionyl]-erythromycin A (9E)-oxime,
4″-O-[(2-{[3-(6-carboxy-3,3-dimethyl-7-oxo-1H,7H-[1,3]oxazino[5,4,3-ij]quinolin-9-yl)propyl]amino}propionyl]-erythromycin A (9E)-methoxymethyl oxime,
4″-O-[(2-{[3-(7-carboxy-8-oxo-3,4-dihydro-2H,8H-[1,4]oxazepino[2,3,4-ij]quinolin-10-yl)propyl]amino}propionyl]-6-O-methyl erythromycin A,
4″-O-[(2-{[3-(7-carboxy-8-oxo-3,4-dihydro-2H,8H-[1,4]oxazepino[2,3,4-ij]quinolin-10-yl)propyl]amino}propionyl]-erythromycin (9E)-methoxime,
4″-O-[(2-{[3-(7-carboxy-8-oxo-3,4-dihydro-2H,8H-[1,4]oxazepino[2,3,4-ij]quinolin-10-yl)propyl]amino}propionyl]-azithromycin-11,12-carbonate,
4″-O-[(2-{[3-(7-carboxy-8-oxo-3,4-dihydro-2H,8H-[1,4]oxazepino[2,3,4-ij]quinolin-10-yl)propyl]amino}propionyl]-azithromycin,
4″-O-[(2-{[3-(7-carboxy-8-oxo-3,4-dihydro-2H,8H-[1,4]oxazepino[2,3,4-ij]quinolin-10-yl)propyl]amino}propionyl]-erythromycin (9E)-oxime,
4″-O-[(2-{([3-(7-carboxy-8-oxo-3,4-dihydro-2H,8H-[1,4]oxazepino[2,3,4-ij]quinolin-10-yl)propyl]amino}propionyl]-erythromycin (9E)-methoxymethyl-oxime,
4″-O-[(2-{[3-(6-carboxy-3,3-dimethyl-7-oxo-2,3-dihydro-7H-[1,4]oxazino[2,3,4-ij]quinolin-9-yl)propyl]amino}propionyl]-erythromycin (9E)-methoxymethyl-oxime,
4″-O-[(2-{[3-(6-carboxy-3,3-dimethyl-7-oxo-2,3-dihydro-7H-[1,4]oxazino[2,3,4-ij]quinolin-9-yl)propyl]amino}propionyl]-6-O-methyl erythromycin A,
4″-O-[(2-{[3-(6-carboxy-3,3-dimethyl-7-oxo-2,3-dihydro-7H-[1,4]oxazino[2,3,4-ij]quinolin-9-yl)propyl]amino}propionyl]-erythromycin A (9E)-methoxime,
4″-O-[(2-{[3-(6-carboxy-3,3-dimethyl-7-oxo-2,3-dihydro-7H-[1,4]oxazino[2,3,4-ij]quinolin-9-yl)propyl]amino}propionyl]-azithromycin,
4″-O-{2-[3-(6-carboxy-3,3-dimethyl-7-oxo-1H,7H-2-[1,3]oxazino[5,4,3-ij]quinolin-9-yl)thio)ethoxy]ethylcarbamoyl}-erythromycin A 9(E)-methyloxime trifluoroacetic acid salt,
O-[(2-{[3-(6-carboxy-3,3-dimethyl-7-oxo-1H,7H-[1,3]oxazino[5,4,3-ij]quinolin-9-yl)propyl]methylamino}propionyl]-6-O-methyl erythromycin A,
4″-O-[(2-{[3-(6-carboxy-3,3-dimethyl-7-oxo-1H,7H-[1,3]oxazino[5,4,3-q′]quinolin-9-yl)propyl]methylamino}ethyl]-6-O-methyl erythromycin A,
4″-O-{3-[2-{[6-carboxy-7-oxo-2,3-dihydro-7H-[1,4]oxazino[2,3,4-ij]quinolin-9-yl]thio}ethylamino]propionyl}-erythromycin A 9(E)-oxime,
4″-O-[3-{3-(7-carboxy-3,4-dihydro-8-oxo-1H,8H-[1,4]oxazepino[6,5,4,-ij]quinolin-10-yl)propylamino}propionyl]-6-O-methyl erythromycin A,
4″-O-[3-{3-(7-carboxy-3,4-dihydro-8-oxo-1H,8H-[1,4]oxazepino[6,5,4,-ij]quinolin-10-yl)propylamino}propionyl]erythromycin A (9E)-methoxymethyl oxime,
4″-O-{3-[2-{[7-carboxy-8-oxo-3,4-dihydro-2H,8H-[1,4]oxazepino[2,3,4-ij]quinolin-10-yl]thio}ethylamino]propionyl}-erythromycin A (9E)-methoxymethyloxime,
4″-O-{3-[2-{[7-carboxy-8-oxo-3,4-dihydro-2H,8H-[1,4]oxazepino[2,3,4-ij]quinolin-10-yl]thio}ethylamino]propionyl}-erythromycin A (9E)-oxime,
4″-O-{2-[2-(6-carboxy-3,3-dimethyl-7-oxo-1H,7H-2-[1,3]oxazino[5,4,3-ij]quinolin-9-yl)thio)ethoxy]ethylcarbamoyl}-erythromycin A 9(E)-(2-diethylaminoethyl)oxime,
4″-O-{2-[3-(6-carboxy-3,3-dimethyl-7-oxo-1H,7H-2-[1,3]oxazino[5,4,3-ij]quinolin-9-yl)propyloxy]ethylcarbamoyl}-erythromycin A 9(E)-cyanomethyloxime,
4″-O-{2-[3-(6-carboxy-7-oxo-1H,7H-2-[1,3]oxazino[5,4,3-ij]quinolin-9-yl)propyloxy]ethylcarbamoyl}-erythromycin A 9(E)-(diethylaminoethyl)oxime,
4″-O-{2-[3-(6-carboxy-7-oxo-1H,7H-2-[1,3]oxazino[5,4,3-ij]quinolin-9-yl)propyloxy]ethylcarbamoyl}-erythromycin A 9(E)-methoxymethyloxime,
4″-O-{2-[3-(6-carboxy-7-oxo-1H,7H-2-[1,3]oxazino[5,4,3-ij]quinolin-9-yl)propyloxy]ethylcarbamoyl}-erythromycin A 9(E)-methyloxime,
4″-O-{2-[2-(6-carboxy-7-oxo-1H,7H-2-[1,3]oxazino[5,4,3-ij]quinolin-9-yl)-propyloxy]ethylcarbamoyl}-erythromycin A 9(E)-oxime,
4″-O-{2-[3-(6-carboxy-7-oxo-1H,7H-2-[1,3]oxazino[5,4,3-ij]quinolin-9-yl)propyloxy]ethylcarbamoyl}-erythromycin A 9(E)-(cyanomethyl)oxime,
4″-O-{2-[3-(6-carboxy-3,3-dimethyl-7-oxo-1H,7H-2-[1,3]oxazino[5,4,3-ij]quinolin-9-yl)propyloxy]ethylcarbamoyl}-erythromycin A 9(E)-methoxymethyloxime,
4″-O-{2-[3-(6-carboxy-3,3-dimethyl-7-oxo-1H,7H-2-[1,3]oxazino[5,4,3-ij]quinolin-9-yl)propyloxy]ethylcarbamoyl}-erythromycin A 9(E)-methyloxime,
4″-O-{2-[3-(6-carboxy-3,3-dimethyl-7-oxo-1H,7H-2-[1,3]oxazino[5,4,3-ij]quinolin-9-yl)propyloxy]ethylcarbamoyl}-6-O-methyl-erythromycin A,
4″-O-{2-[3-(6-carboxy-3,3-dimethyl-7-oxo-1H,7H-2-[1,3]oxazino[5,4,3-ij]quinolin-9-yl)propyloxy]ethylcarbamoyl}-erythromycin A 9(E)-(2-diethylaminoethyl)oxime,
4″-O-{2-[3-(6-carboxy-3,3-dimethyl-7-oxo-1H,7H-2-[1,3]oxazino[5,4,3-ij]quinolin-9-yl)propyloxy]ethylcarbamoyl}-erythromycin A 9(E)-oxime,
4″-O-{2-[3-(6-carboxy-3,3-dimethyl-7-oxo-1H,7H-2-[1,3]oxazino[5,4,3-ij]quinolin-9-yl-propyloxy]-ethylcarbamoyl}-(9S)-9-O-11-O-ethylidene-9-dihydroerythromycin A,
4″-O-{2-[2-(6-carboxy-3,3-dimethyl-7-oxo-1H,7H-2-[1,3]oxazino[5,4,3-ij]quinolin-9-yl)thio)ethoxy]ethylcarbamoyl}-erythromycin A 9(E)-oxime trifluoroacetic acid salt, and
4″-O-{2-[3-(6-carboxy-3,3-dimethyl-7-oxo-1H,7H-2-[1,3]oxazino[5,4,3-ij]quinolin-9-yl)thio)ethoxy]-ethylcarbamoyl}-erythromycin A 9(E)-methoxymethyloxime trifluoroacetic acid salt,
   and pharmaceutically acceptable derivatives thereof.

Compounds of the invention also include:
4″-O-[(2-{[3-(6-carboxy-3,3-dimethyl-7-oxo-1H,7H-[1,3]oxazino[5,4,3-ij]quinolin-9-yl)propyl]amino}propionyl]-6-O-methyl erythromycin A;
4″-O-[(2-{[3-(7-carboxy-8-oxo-3,4-dihydro-2H,8H-[1,4]oxazepino[2,3,4-ij]quinolin-10-yl)propyl]amino}propionyl]-6-O-methyl erythromycin A;
4″-O-[(2-{[3-(7-carboxy-8-oxo-3,4-dihydro-2H,8H-[1,4]oxazepino[2,3,4-ij]quinolin-10-yl)propyl]amino}propionyl]-erythromycin (9E)-methoxime;
4″-O-[(2-{[3-(7-carboxy-8-oxo-3,4-dihydro-2H,8H-[1,4]oxazepino[2,3,4-ij]quinolin-10-yl)propyl]amino}propionyl]-erythromycin (9E)-oxime;
4″-O-[(2-{[3-(7-carboxy-8-oxo-3,4-dihydro-2H,8H-[1,4]oxazepino[2,3,4-ij]quinolin-10-yl)propyl]amino}propionyl]-erythromycin (9E)-methoxymethyl oxime;
4″-O-[(2-{[3-(6-carboxy-3,3-dimethyl-7-oxo-2,3-dihydro-7H-[1,4]oxazino[2,3,4-ij]quinolin-9-yl)propyl]amino}propionyl]-erythromycin A (9E)-methoxime;
   and pharmaceutically acceptable derivatives thereof.

One or more compounds according to the invention exhibit antimicrobial activity, in particular antibacterial activity, against a range of clinical pathogenic microorganisms. Using a standard microtiter broth serial dilution test, one or more compounds of the invention have been found to exhibit useful levels of activity against a range of pathogenic microorganisms. For example, the compounds of the invention may be active against strains of Staphylococcus aureus, Streptopococcus pneumoniae, Moraxella catarrhalis, Streptococcus pyogenes, Haemophilus influenzae, Enterococcus faecalis, Chlamydia pneumoniae, Mycoplasma pneumoniae and Legionella pneumophila. The compounds of the invention may also be active against resistant strains, for example erythromycin resistant strains. Thus, for example, the compounds of the invention may be active against erythromycin resistant strains of Streptococcus pneumoniae, Streptococcus pyogenes and Staphylococcus aureus.

The compounds of the invention may therefore be used for treating a variety of diseases caused by pathogenic microorganisms, in particular bacteria, in human beings and animals. It will be appreciated that reference to treatment includes acute treatment or prophylaxis as well as the alleviation of established symptoms.

However, it will appreciated by person skilled in the art that compounds of the invention may have different levels of activity against different strains of the same bacteria.

Thus, according to another aspect of the present invention we provide a compound of formula (I) or a pharmaceutically acceptable derivative thereof for use in therapy.

According to a further aspect of the invention we provide a compound of formula (I) or a pharmaceutically acceptable derivative thereof for use in the treatment or prophylaxis of systemic or topical microbial infections in a human or animal body.

According to a further aspect of the invention we provide the use of a compound of formula (I) or a pharmaceutically acceptable derivative thereof in the manufacture of a medicament for use in the treatment or prophylaxis of systemic or topical microbial infections in a human or animal body.

According to a yet further aspect of the invention we provide a method of treatment of the human or non-human animal body to combat microbial infections comprising administration to a body in need of such treatment of an effective amount of a compound of formula (I) or a pharmaceutically acceptable derivative thereof.

While it is possible that, for use in therapy, a compound of the invention may be administered as the raw chemical it is preferable to present the active ingredient as a pharmaceutical formulation eg when the agent is in admixture with a suitable pharmaceutical excipient, such as a diluent or carrier selected with regard to the intended route of administration and standard pharmaceutical practice.

Accordingly, in one aspect, the present invention provides a pharmaceutical composition or formulation comprising a compound of the invention or a pharmaceutically acceptable derivative thereof in association with a pharmaceutically acceptable excipient such as a diluent and/or carrier. The excipient, such as a diluent and/or carrier, must be “acceptable” in the sense of being compatible with the other ingredients of the formulation and not deleterious to the recipient thereof.

In another aspect, the invention provides a pharmaceutical composition comprising, as active ingredient, a compound of the invention or a pharmaceutically acceptable derivative thereof in association with a pharmaceutically acceptable excipient such as a diluent and/or carrier for use in therapy, and in particular, in the treatment of human or animal subjects suffering from a condition susceptible to amelioration by an antimicrobial compound.

In another aspect, the invention provides a pharmaceutical composition comprising a therapeutically effective amount of the compounds of the present invention, (including combinations thereof) and a pharmaceutically acceptable excipient such as a diluent and/or carrier.

There is further provided by the present invention a process of preparing a pharmaceutical composition, which process comprises mixing a compound of the invention or a pharmaceutically acceptable derivative thereof, together with a pharmaceutically acceptable excipient, diluent and/or carrier.

The compounds of the invention may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable excipients, such as diluents and/or carriers. Acceptable excipients, such as diluents and carriers, for therapeutic use are well known in the pharmaceutical art, and are described, for example, in Remington's Pharmaceutical Sciences, Mack Publishing Co. (A. R. Gennaro edit. 1985). The choice of pharmaceutical excipient, such as diluent and/or carrier, can be selected with regard to the intended route of administration and standard pharmaceutical practice. The pharmaceutical compositions may comprise as—or in addition to—the excipient, any suitable binder(s), lubricant(s), suspending agent(s), coating agent(s), and/or solubilising agent(s), as required.

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO 91/11172, WO 94/02518 and WO 98/55148.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see International Patent Application No. WO 02/00196 (SmithKline Beecham).

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual.

There may be different composition/formulation requirements depending on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by: inhalation; in the form of a suppository or pessary; topically in the form of a lotion, solution, cream, ointment or dusting powder; transdermally, for example, by use of a skin patch; orally in the form of tablets containing excipients such as starch or lactose, in capsules or ovules either alone or in admixture with excipients, in the form of elixirs, solutions or suspensions containing flavouring or colouring agents; or parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

It is to be understood that not all of the compounds need be administered by the same route. Likewise, if the composition comprises more than one active component, then those components may be administered by different routes.

The compositions of the invention include those in a form especially formulated for parenteral, oral, buccal, rectal, topical, implant, ophthalmic, nasal or genito-urinary use. For some applications, the agents of the present invention are delivered systemically (such as orally, buccally, sublingually), more preferably orally. Hence, preferably the agent is in a form that is suitable for oral delivery.

If the compound of the present invention is administered parenterally, then examples of such administration include one or more of: intravenously, intraarterially, intraperitoneally, intrathecally, intraventricularly, intraurethrally, intrasternally, intracranially, intramuscularly or subcutaneously administering the agent, and/or by using infusion techniques.

For parenteral administration, the compound is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The compounds according to the invention may be formulated for use in human or veterinary medicine by injection (eg by intravenous bolus injection or infusion or via intramuscular, subcutaneous or intrathecal routes) and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilising, solubilising and/or dispersing agents. Alternatively the active ingredient may be in sterile powder form for reconstitution with a suitable vehicle, eg sterile, pyrogen-free water, before use.

The compounds of the invention can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

The compounds of the invention may also be presented for human or veterinary use in a form suitable for oral or buccal administration, for example in the form of solutions, gels, syrups, mouth washes or suspensions, or a dry powder for constitution with water or other suitable vehicle before use, optionally with flavouring and colouring agents. Solid compositions such as tablets, capsules, lozenges, pastilles, pills, boluses, powder, pastes, granules, bullets or premix preparations may also be used. Solid and liquid compositions for oral use may be prepared according to methods well known in the art. Such compositions may also contain one or more pharmaceutically acceptable carriers and optionally other excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The compounds of the invention may also be administered orally in veterinary medicine in the form of a liquid drench such as a solution, suspension or dispersion of the active ingredient together with a pharmaceutically acceptable carrier or excipient.

The compounds of the invention may also, for example, be formulated as suppositories eg containing conventional suppository bases for use in human or veterinary medicine or as pessaries eg containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (eg eye ear or nose drops) or pour-ons.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water.

Alternatively, it can be formulated as a suitable lotion or cream, suspended or dissolved in, for example, as a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds may also be dermally or transdermally administered, for example, by use of a skin patch.

For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

As indicated, the compound of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134AT′″) or 1,1,1,2,3,3,3-heptafluoropropane (HFA 227EA), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, eg using a mixture of ethanol and the propellant as the solvent, which may additionally contain a lubricant, e.g. sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebuliser.

The compounds of the invention may also be used in combination with other therapeutic agents. The invention thus provides, in a further aspect, a combination comprising a compound of the invention or a pharmaceutically acceptable derivative thereof together with a further therapeutic agent.

When a compound of the invention or a pharmaceutically acceptable derivative thereof is used in combination with a second therapeutic agent active against the same or different disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the present invention may, for example, be used for topical administration with other active ingredients such as corticosteroids or antifungals as appropriate.

The individual components of such combinations may be administered either sequentially or simultaneously in separate or combined pharmaceutical formulations, by any convenient route. However, the combinations referred to above may conveniently be presented for use in the form of a pharmaceutical formulation and thus pharmaceutical formulations comprising a combination, as defined above, together with a pharmaceutically acceptable excipient such as a carrier, comprises a further aspect of the invention.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, as are known for such compounds in the art.

The compositions may contain from 0.01-99% of the active material. For topical administration, for example, the composition will generally contain from 0.01-10%, more preferably 0.01-1% of the active material.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

For oral and parenteral administration to humans, the daily dosage level of the agent may be in single or divided doses.

For systemic administration the daily dose as employed for adult human treatment it will range from 2-100 mg/kg body weight, preferably 5-60 mg/kg body weight, which may be administered in 1 to 4 daily doses, for example, depending on the route of administration and the condition of the patient. When the composition comprises dosage units, each unit will preferably contain 200 mg to 1 g of active ingredient. The duration of treatment will be dictated by the rate of response rather than by arbitrary numbers of days.

Compounds of general formula (I) and pharmaceutically acceptable derivatives thereof may be prepared by the general methods outlined hereinafter, said methods constituting a further aspect of the invention. In the following description, the groups R¹ to R³⁹, U¹, U², V, W, X, Y, Z, d, e, f, g, h, i, j, k, m, n, p, q, r, s and t have the meaning defined for the compounds of formula (I) unless otherwise stated.

The groups U^{1z}R^{14z}, U^{2z}R^{14z}, X^{z}R^{14z} and R^{14z} are U¹R¹⁴, U²R¹⁴, XR¹⁴ and R¹⁴ as defined for formula (I) or groups convertible to U¹R¹⁴, U²R¹⁴, XR¹⁴ and R¹⁴. Conversion of a group U^{1z}R^{14z}, U^{2z}R^{14z}, X^{z}R^{14z} or R^{14z} to a U¹R¹⁴, U²R¹⁴, XR¹⁴ or R¹⁴ group typically arises if a protecting group is needed during the reactions described below. A comprehensive discussion of the ways in which such groups may be protected and methods for cleaving the resulting protected derivatives is given by for example T. W. Greene and P. G. M Wuts in Protective Groups in Organic Synthesis 2^{nd} ed., John Wiley & Son, Inc 1991 and by P. J. Kocienski in Protecting Groups, Georg Thieme Verlag 1994 which are incorporated herein by reference. Examples of suitable amino protecting groups include acyl type protecting groups (eg formyl, trifluoroacetyl and acetyl), aromatic urethane type protecting groups (eg benzyloxycarbonyl (Cbz) and substituted Cbz, and 9-fluorenylmethoxycarbonyl (Fmoc)), aliphatic urethane protecting groups (eg t-butyloxycarbonyl (Boc), isopropyloxycarbonyl and cyclohexyloxycarbonyl) and alkyl type protecting groups (eg benzyl, trityl and chlorotrityl). Examples of suitable oxygen protecting groups may include for example alkyl silyl groups, such as trimethylsilyl or tert-butyldimethylsilyl; alkyl ethers such as tetrahydropyranyl or tert-butyl; or esters such as acetate. Hydroxy groups may be protected by reaction of for example acetic anhydride, benzoic anhydride or a trialkylsilyl chloride in an aprotic solvent. Examples of aprotic solvents are dichloromethane, N,N-dimethylformamide, dimethylsulfoxide, tetrahydrofuran and the like.

The compounds of general formula (I) and derivatives thereof may be purified by conventional methods known in the art. For example, the compounds may be purified by HPLC using an aqueous solution of an acid such as formic acid with an organic co-solvent such as acetonitrile or methanol.

In one embodiment of the invention, compounds of formula (I) wherein R¹³ is —OC(O)(CH₂)_{d}U¹R¹⁴ and d is an integer from 2 to 5 may be prepared by reaction of a 4″ hydroxy compound of formula (II) wherein R² may be a hydroxy protecting group with a suitable activated and protected derivative of the carboxylic acid (III), followed where necessary by subsequent removal of the hydroxyl protecting group R² and conversion of the U^{1z}R^{14z} group to U¹R¹⁴.

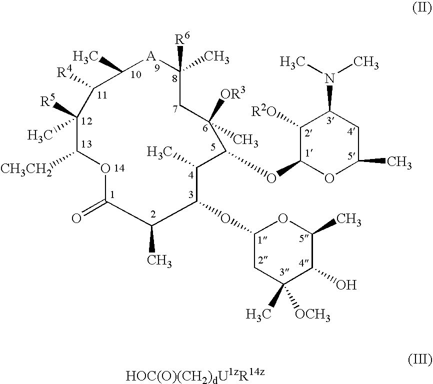

Compounds of formula (I) wherein R¹³ is —NHC(O)(CH₂)_{d}U¹R¹⁴ and d is an integer from 2 to 5 may be prepared by reaction of a 4″ amine compound of formula (IIA) with a carboxylic acid compound of formula (IIIA), or a suitable activated and protected derivative thereof, followed where necessary by subsequent conversion of the U^{1z}R^{14z} group to U¹R¹⁴.

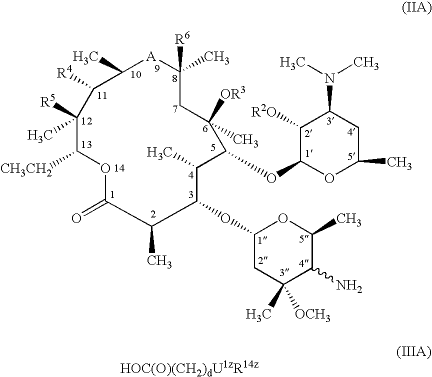

Suitable activated derivatives of the carboxyl group in the compounds of formula (III) or (IIIA) include the corresponding acyl halide, mixed anhydride or activated ester such as a thioester. The reaction is preferably carried out in a suitable aprotic solvent such as a halohydrocarbon (eg dichloromethane) or N,N-dimethylformamide optionally in the presence of a tertiary organic base such as dimethylaminopyridine or triethylamine or in the presence of inorganic base (eg sodium hydroxide) and at a temperature within the range of 0° to 120° C. The compounds of formula (II) or (IIA) and (III) or (IIIA) may also be reacted in the presence of a carbodiimide such as dicyclohexylcarbodiimide (DCC).

In another embodiment of the invention, compounds of formula (I) wherein R¹³ is —OC(O)N(R¹⁵)(CH₂)_{d}U¹R¹⁴, 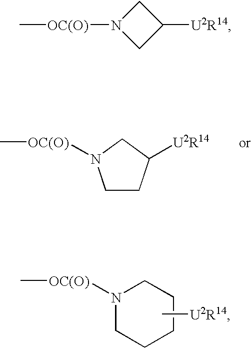
may be prepared by reaction of a suitable activated compound of formula (IIB) wherein R² is optionally a hydroxy protecting group and R⁴⁰ is an activating group such as imidazolyl or halogen, with a suitable protected derivative of an amine (IV), (IVA), (IVB) or (IVC), followed where necessary by subsequent removal of the hydroxyl protecting group R² and conversion of the U^{1z}R^{14z} or U^{2z}R^{14z} group to U¹R¹⁴ or U²R¹⁴.

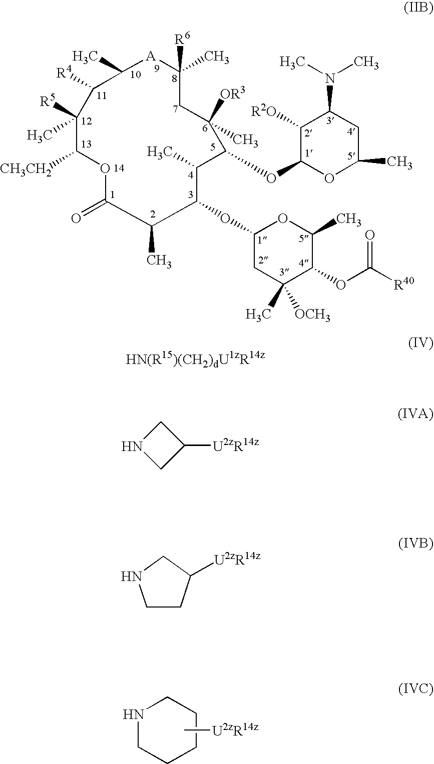

The reaction is preferably carried out in a suitable aprotic solvent such as N,N-dimethylformamide in the presence of an organic base such as 1,8-diazabiyclo[5.4.0]undec-7-ene (DBU).

In another embodiment of the invention, compounds of formula (I) wherein R¹³ is —O(CH₂)_{d}U¹R¹⁴, U¹ is —W(CH₂)_{q}X— or —W(CH₂)_{q}—, and W is —N(R³⁹)— may be prepared by reaction of a 4″ aldehyde compound of formula (IIC) wherein A, R⁴ and R⁵ may be suitably protected and d′ is an integer from 1 to 4, with a suitable protected derivative of the amine (V) or (VA), followed where necessary by subsequent removal of the hydroxyl protecting group R² and conversion of the X^{z}R^{14z} or R^{14z} group to XR¹⁴ or R¹⁴.

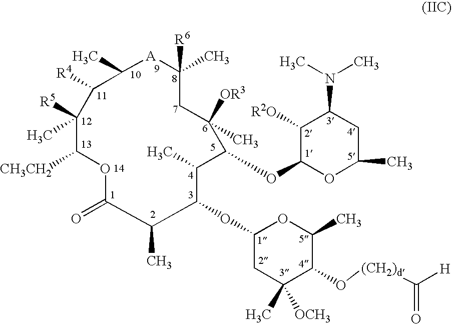

The reductive amination reaction is preferably carried out in a solvent such as methanol and DMF under neutral pH to mildly acidic conditions. A suitable reducing agent is, for example, sodium cyanoborohydride, and suitable reagents for adjusting acidity are acetic acid and sodium acetate.

Compounds of formula (IIC) where d′ is 1 may be prepared from suitably protected compounds of formula (VI) by oxidative cleavage for example using osmium tetroxide and sodium periodate. Where d′ is 2, hydroboration of suitably protected compounds of formula (VI) with 9-BBN, or other suitable boranes, followed by treatment with peroxide and then oxidation yields compounds of formula (IIC), d′ is 2. For d′=3 or 4, compounds of formula (VI) may be chain extended using olefin cross-metathesis (H. E. Blackwell et al. J. Am. Chem. Soc., 2000, 122, 58-71) with a suitably functionalised olefin, for example but-2-ene-1,4-diol, followed by double bond reduction and oxidation of the terminal alcohol. Compounds of formula (VI) can be formed by palladium-catalysed allylation of suitably protected 4″ hydroxy compounds, for example when A is —C(O)—, by 2′,11-bis-silylation and conversion of the 9-ketone to a bicyclic ketal by interaction with the 12-OH and an alcohol, for example methanol.

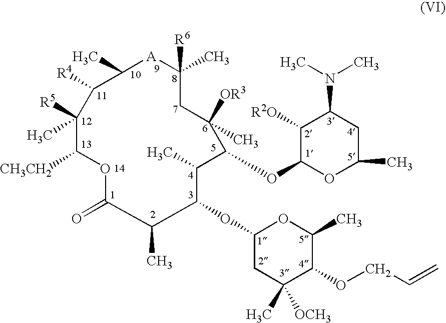

In another embodiment of the invention, compounds of formula (I) wherein R¹³ is —OC(O)(CH₂)_{d}U¹R¹⁴, d is an integer from 2 to 5, U is —W(CH₂)_{q}X—, and W is —N(R³⁹)—, —O— or —S—, may be prepared by reaction of compounds of formula (VII) 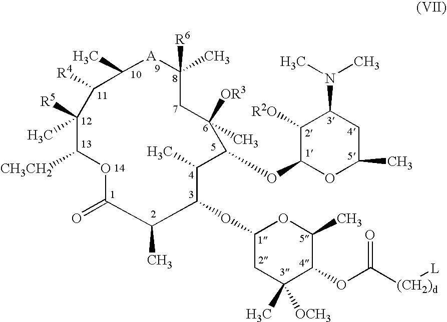
wherein d is an integer from 2 to 5 and L is a suitable leaving group, with HU^{1z}R^{14z} (VIII) in which W is —N(R³⁹)—, —O— or —S—.

Similarly, compounds of formula (I) wherein R¹³ is —OC(O)N(R¹⁵)(CH₂)_{d}U¹R¹⁴, U is —W(CH₂)_{q}X— or —W(CH₂)_{q}—, and W is —N(R³⁹)— or —S—, may be prepared by reaction of compounds of formula (VIIA) 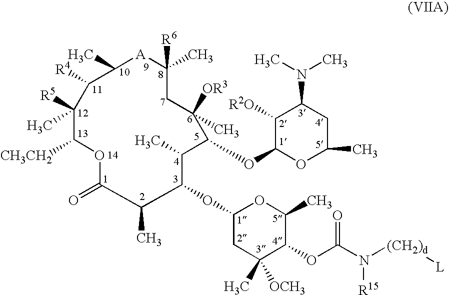
wherein d is an integer from 2 to 5 and L is a suitable leaving group, with HU^{1z}R^{14z} (VIII) in which W is —N(R³⁹)— or —S—.

Similarly, compounds of formula (I) wherein R¹³ is —O(CH₂)_{d}U¹R¹⁴, U¹ is —W(CH₂)₁X— or formula (VIIB) 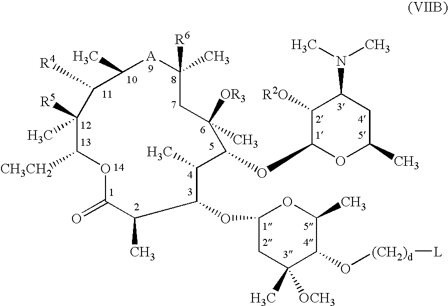
wherein d is an integer from 2 to 5 and L is a suitable leaving group, with HU^{1z}R^{14z} (VIII) in which W is —N(R³⁹)— or —S—.

Further, compounds of formula (I) wherein R¹³ is —NHC(O)(CH₂)_{d}U¹R¹⁴, d is an integer from 2 to 5, U¹ is —W(CH₂)_{q}X— or —W(CH₂)_{q}—, and W is —N(R³⁹)— or —S—, may be prepared by reaction of compounds of formula (VIIC) 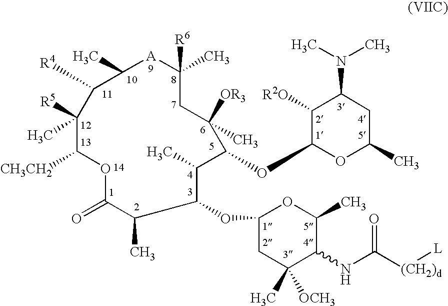
wherein d is an integer from 2 to 5 and L is a suitable leaving group, with HU^{1z}R^{14z} (VIII) in which W is —N(R³⁹)— or —S—.

The reaction between (VII), (VIIA), (VIIB) or (VIIC) and (VIII) is preferably carried out in a solvent such as a halohydrocarbon (eg dichloromethane), an ether (eg tetrahydrofuran or dimethoxyethane), acetonitrile or ethyl acetate and the like, dimethylsulfoxide, N,N-dimethylformamide or 1-methyl-pyrrolidinone and in the presence of a base, followed, if desired, by removal of the hydroxyl protecting group R² and conversion of the U^{1z}R^{14z} group to U¹R¹⁴. Examples of the bases which may be used include organic bases such as diisopropylethylamine, triethylamine and 1,8-diazabicyclo[5.4.0]undec-7-ene, and inorganic bases such as potassium hydroxide, cesium hydroxide, tetraalkylammonium hydroxide, sodium hydride and potassium hydride. Suitable leaving groups for this reaction include halide (eg chloride, bromide or iodide) or a sulfonyloxy group (eg tosyloxy or methanesulfonyloxy).

Compounds of formula (VII) and (VIIC) may be prepared by reaction of a compound of formula (II) or (IIA), wherein R² is a hydroxyl protecting group, with a suitable activated derivative of the carboxylic acid HOC(O)(CH₂)_{d}L (IX), wherein L is a suitable leaving group as above defined. Suitable activated derivatives of the carboxyl group are those defined above for carboxylic acids (III) or (IIIA). The reaction is carried out using the conditions described above for the reaction of a compound of formula (II) or (IIA) with carboxylic acid (III) or (IIIA).

In another embodiment of the invention, compounds of formula (I) wherein R¹³ is —O(CH₂)_{d}U¹R¹⁴, U¹ is —O(CH₂)_{q}X— or —O(CH₂)_{q}—, may be prepared by reaction of compounds of formula (X) 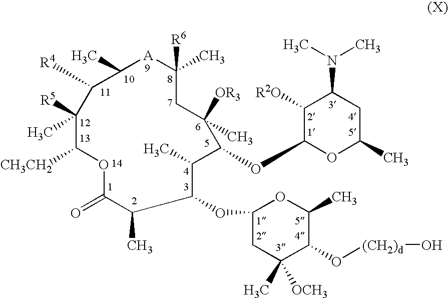
wherein d is an integer from 2 to 5 with a suitable compound of formula HU^{1z}R^{14z} (VIII), for example a compound of formula (XI) 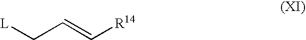
in which L is a suitable leaving group, in the presence of a catalyst such as tetrakis(triphenylphosphine)palladium.

In one embodiment of the invention, compounds of formula (I) wherein R¹³ is —OC(O)(CH₂)_{d}U¹R¹⁴, d is 2, U¹ is as above defined, and W is —N(R³⁹)— or —S—, may be prepared by Michael reaction of a compound of formula (XII), wherein R² is optionally a hydroxyl protecting group 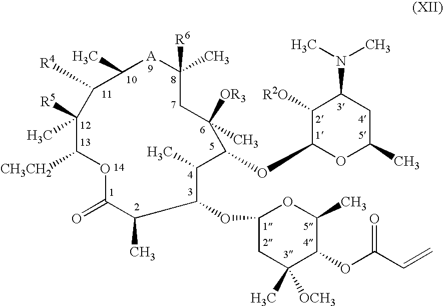
with a compound of formula HU^{1z}R^{14z} (VIII). The Reaction is Suitably Carried Out in a solvent such as dimethylsulfoxide, N,N-dimethylformamide, 1-methyl-pyrrolidinone, a halohydrocarbon (eg dichloromethane), an ether (eg tetrahydrofuran or dimethoxyethane), acetonitrile or alcohol (e.g methanol or isopropanol) and the like, and in the presence of a base, followed, if desired, by removal of hydroxyl protecting group R² and conversion of the U^{1z}R^{14z} group to U¹R¹⁴. Similarly, compounds of formula (I) wherein R¹³ is —OC(O)(CH₂)_{d}U¹R¹⁴, d is 2, U¹ is as above defined and Y is —O— may also be prepared by Michael reaction in a solvent such as dimethylsulfoxide, N,N-dimethylformamide, 1-methyl-pyrrolidinone, a halohydrocarbon (eg dichloromethane), an ether (eg tetrahydrofuran or dimethoxyethane) or acetonitrile, and in the presence of a base.

Compounds of formula (I) may be converted into other compounds of formula (I). Thus, for example, compounds of formula (I) wherein W is —S(O)ₜ— and t is 1 or 2 may be prepared by oxidation of the corresponding compound of formula (I) wherein t is 0. The oxidation is preferably carried out using a peracid, eg peroxybenzoic acid, followed by treatment with a phosphine, such as triphenylphosphine. The reaction is suitably carried out in an organic solvent such as methylene chloride. Compounds of formula (I) wherein, for example, W is —N(R³⁹)— and R³⁹ is C₁₋₄alkyl can be prepared from compounds wherein R³⁹ is hydrogen by reductive alkylation. Compounds of formula (I) wherein Y is —N(R³⁹)— and R³⁹ is acetyl or benzoyl can be prepared from compounds wherein R³⁹ is hydrogen by acylation.

Compounds of formula (II), (IIA) and (IIB), wherein A is —C(O)—, —N(R⁷)—CH₂— or —CH(NR⁸R⁹)—, R⁴ or R⁵ are hydroxy or R⁴ and R⁵ taken together with the intervening atoms form a cyclic group having the following structure: 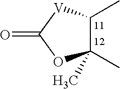
wherein V is a bivalent radical selected from —O— and —N(R¹⁷)—, and R³ is C₁₋₄alkyl, or C₃₋₆alkenyl optionally substituted by 9- or 10-membered fused bicyclic heteroaryl are known compounds or they may be prepared by analogous methods to those known in the art. Thus they can be prepared according to the procedures described in EP 0 307 177, EP 0 248 279, WO 00/78773 and WO 97/42204.

Compounds of formula (II), (IIA), (IIB) and (IIC) wherein A is —N(CH₃)—CH₂—, R⁴ or R⁵ are hydroxy or R⁴ and R⁵ taken together with the intervening atoms form a cyclic group having the following structure: 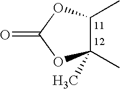
and R⁶ is hydrogen are known compounds or they may be prepared by analogous methods to those known in the art, such as the procedures described in EP 0 508 699, J. Chem. Res. Synop. (1988, pages 152-153) and U.S. Pat. No. 6,262,030.

Compounds of formula (II), (IIA) and (IIB), wherein A is —C(═NR¹⁰)—, R⁴ or R⁵ are hydroxy or R⁴ and R⁵ taken together with the intervening atoms form a cyclic group having the following structure: 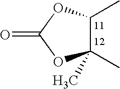
and R⁶ is hydrogen, are known compounds or they may be prepared by analogous methods to those known in the art, such as the procedures described in EP 0 284 203. Compounds of formula (II), (IIA), (IIB) and (IIC) wherein A is —C(O)—, R⁴ and R⁵ taken together with the intervening atoms form a cyclic group having the following structure: 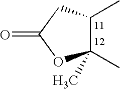
R⁶ is hydrogen and R³ is C₁₋₄alkyl may be prepared by decarboxylation of a compound of formula (XIII), wherein R⁴¹ is a hydroxy protecting group followed, if required, by removal of the protecting group R² or R⁴¹.

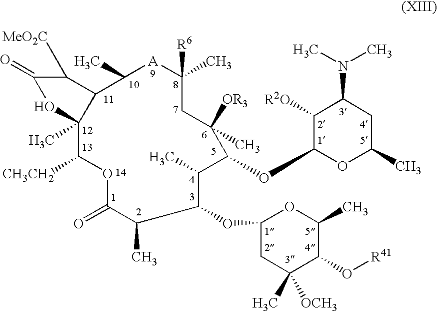

The decarboxylation may be carried out in the presence of a lithium salt such as lithium chloride, usually in an organic solvent such as dimethylsulfoxide.

Compounds of formula (II), (IIA), (IIB) and (IIC) wherein A is —C(O)—, R⁴ and R⁵ taken together with the intervening atoms form a cyclic group having the following structure: 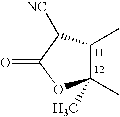
and R³ is C₁₋₄alkyl may be prepared according to the procedures described in WO 02/50091 and WO 02/50092.

Compounds of formula (III) and (IIIA) wherein U¹ is —W(CH₂)_{q}N(R³⁹)— or —W(CH₂)_{q}—, wherein W is —N(R³⁹)—, —O— or —S—, may be prepared by reaction of HU^{1z}R^{14z} (VIII), wherein U^{1z} has the meaning defined above with R⁴²OC(O)(CH₂)_{d}L (XIV) wherein R⁴² is carboxyl protecting group and L is a suitable leaving group, followed by removal of R⁴². Suitable R⁴² carboxyl protecting groups include t-butyl, allyl or benzyl.

Compounds of formula (III) and (IIIA) may also be prepared by reaction of HU^{1z}R^{14z} (VIII) with acrylonitrile followed by hydrolysis of the nitrile to the acid, or by reaction of HU^{1z}R^{14z} (VIII) with t-butyl acrylate followed by removal of the t-butyl group.

Compounds of formula (VIII) wherein U¹ is —W(CH₂)_{q}X— in which X is —N(R³⁹)—, —O— or —S—, may be prepared by reaction of a compound of formula R^{14z}L (XV), wherein L is a suitable leaving group such as chlorine, fluorine or bromine, with a compound of formula —W(CH₂)_{q}X— (XVI) in which X is —N(R³⁹)—, —O— or —S—.

Compounds of formula (I) wherein R¹³ is —O(CH₂)_{d}U¹R¹⁴, U¹ is —W(CH₂)_{q}X— or —W(CH₂)_{q}—, and W is —C(O)N(R³⁹)—, may be prepared by reaction of compounds of formula (XVII) 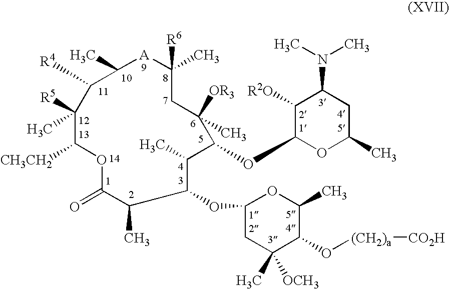
with a suitable amine compound.

All publications, including but not limited to patents and patent applications, cited in this specification are herein incorporated by reference as if each individual publication were specifically and individually indicated to be incorporated by reference herein as though fully set forth.

In order that the invention may be more fully understood the following examples are given by way of illustration only.

### EXAMPLES

The following abbreviations are used in the text: 9-BBN for 9-borabicyclo[3.3.1]nonane, BOC for t-butoxycarbonyl, DBU for 1,8-diazabicyclo[5.4.0]undec-7-ene, DCM for dichloromethane, DMF for N,N-dimethylformamide, DMSO for dimethyl sulfoxide, EtOAc for ethyl acetate, EtOH for ethanol, MeCN for acetonitrile, MeOH for methanol, TFA for trifluoroacetic acid, THF for tetrahydrofuran, MgSO₄ for anhydrous magnesium sulphate, Na₂SO₄ for anhydrous sodium sulphate, TFA for trifluoro acetic acid, and SCX chromatography for strong cation exchange chromatography.

2′-O-Acetyl-6-O-methyl-erythromycin A may be prepared by the procedure described by W. R. Baker et al. in J. Org. Chem. 1988, 53, 2340 and 2′-O-acetyl-azithromycin-11,12-carbonate may be prepared by the procedure described by S. Djokic et al. in J. Chem. Res. (S) 1988, 152.

Erythromycin A (9E)-oxime may be prepared by the procedure described by R. R. Wilkening in EP 0 508 726 A1.

Erythromycin A (9E) methoxime may be prepared by the procedure described by J. R. Everett et al. in J. Chem. Soc. Perkin 2, 1989, 11, 1719-1728.

6-O-Methyl erythromycin A (9E)-oxime may be prepared by the procedure described by R. A. Dominguez et al in US 2003023053.

2′-O-acetyl-azithromycin and 2′-O-Acetyl-azithromycin-11,12-carbonate may be prepared by the procedures described by S. Djokic et al. in J. Chem. Res. (S) 1988, 152.

2′-O-Acetyl-erythromycin A-(9E)-O-acetyl-oxime may be prepared by the procedure described by J Berge et. al. in WO 2004039822.

2′-O-Acetyl-erythromycin A 11,12-carbonate may be prepared by the procedure described by L. Freidberg et. al. in U.S. Pat. No. 4,686,207A.

Erythromycin A-(9E)-O-methoxymethyloxime may be prepared by the procedure described by Gasc, Jean Claude et al. in Journal of Antibiotics., 1991, 44(3), 313-30.

Erythromycin A (9E)-O-(1-methoxy-1-methylethyl)-oxime may be prepared by the procedure described by S. Morimoto et al. in U.S. Pat. No. 4,990,602.

Erythromycin A (9E)-O-(2-diethylaminoethyl)-oxime may be prepared by the procedure described by S. Gouin d'Ambrieres et al. in U.S. Pat. No. 4,349,545.

(9S)-9-O,11-O-Ethylidene-9-dihydroerythromycin A may be prepared by the procedure described by E. Hunt et al. in J. Antibiotics, 1989, 42, 293-298.

Preparative reverse phase HPLC refers to the use of an C18 column with a gradient of MeCN containing 0.1% TFA in water containing 0.1% TFA as eluent.

Mass directed automatic preparative HPLC refers to the use of Waters Atlantis dC18 5 micron columns with a gradient of MeCN containing 0.1% HCO₂H in H₂O containing 0.1% HCO₂H as eluent.

### Intermediate 1: 9-(3-Aminopropyl)-3-methyl-7-oxo-1H,7H-[1,3]oxazino[5,4,3-ij]quinoline-6-carboxylic acid

### a) 2-[(2-Hydroxymethyl-4-iodo-phenylamino)-methylene]malonic acid diethyl ester

A mixture of (2-amino-5-iodo-phenyl)methanol (2.22 g, 8.9 mmol) (C. Alabaster et al., J. Med. Chem., 1988, 31(10), 2048) and diethyl ethoxymethylenemalonate (1.93 g, 8.9 mmol) was heated to 115° C. After 5 h the reaction mixture was concentrated and the residue was chromatographed on silica gel eluting with 0 to 5% [9:1 methanol/20M ammonia] in dichloromethane to give the title compound. (3.43 g) as a beige solid; ESMS m/z 420.0 [M+H]⁺.

### b) 2-[(2-Acetoxymethyl-4-iodo-phenylamino)-methylene]malonic acid diethyl ester

To a solution of Intermediate 1a (14.22 g, 33.9 mmol) in triethylamine (15.5 mL) and dichloromethane (150 mL) was added dropwise a solution of acetyl chloride (4.73 mL) in dichloromethane (45 mL). After 1 h the solution was washed with water, the organic phase dried (MgSO₄), filtered, and concentrated in vacuo to give a residue which was purified by chromatography (silica gel, 0-6% diethyl ether in dichloromethane) to give the title compound (14.58 g) as a pale yellow solid; ESMS m/z 462.1 [M+H]⁺.

### c) 8-Acetoxymethyl-6-iodo-4-oxo-1,4-dihydro-quinoline-3-carboxylic acid ethyl ester

A suspension of Intermediate 1b (14.57 g, 31.6 mmol) and diphenyl ether (200 mL) was heated to 260° C. using a Dean-Stark apparatus. After the collection of ethanol was over the reaction mixture was allowed to cool down. The residue was filtered off, washed with diisopropyl ether then dried in a dessicator to give the title compound (9.8 g) as a grey solid; ESMS m/z 416.0 [M+H]⁺.

### d) 8-Hydroxymethyl-6-iodo-4-oxo-1,4-dihydro-quinoline-3-carboxylic acid ethyl ester

A suspension of Intermediate 1c (1 g, 2.4 mmol) and sodium ethoxide (0.165 g, 2.4 mmol) in ethanol (50 mL) was heated at 80° C. for 2 h. The reaction mixture was allowed to cool down then the product was preabsorbed on silica gel and purified by chromatography eluting with 0 to 25% [9:1 methanol/20M ammonia] in dichloromethane to give the title compound. (0.73 g) as a beige solid; ESMS m/z 374.0 [M+H]⁺.

### e) 9-Iodo-3-methyl-7-oxo-1H,7H-[1,3]oxazino[5,4,3-ij]quinoline-6-carboxylic acid ethyl ester

A mixture of Intermediate 1d (0.50 g, 1.34 mmol), 4-toluenesulfonic acid (0.211 g, 1.11 mmol) and 1,1-diethoxyethane (1.4 mL, 13.4 mmol) in N-methylpyrrolidinone (1 mL) was heated at 80° C. for 7 h. The reaction mixture was allowed to cool and diluted with chloroform (40 mL). The resultant solution was washed with 5% aqueous sodium hydrogen carbonate solution. The organic layer was separated, dried and evaporated to yield the title compound (0.478 g) as a pale yellow solid; ESMS m/z 399.9 [M+H]⁺.

### f) 9-(3-tert-Butoxycarbonylamino-prop-1-ynyl)-3-methyl-7-oxo-1H,7H-[1,3]oxazino[5,4,3-ij]quinoline-6-carboxylic acid ethyl ester

A mixture of Intermediate 1e (0.478 g, 1.19 mmol), copper (I) iodide (0.025 g, 0.13 mmol) and triethylamine (5.8 mL, 42 mmol) were suspended in dry acetonitrile (12 mL). The light green suspension was heated to 50° C. whilst argon was bubbled through. After 20 min, dichlorobis(triphenylphosphine)palladium (II) (0.025 g, 0.036 mmol) and N-tert-butoxycarbonylpropargylamine (0.314 g, 2.02 mmol) were added and the mixture was heated at 50° C. for 2 h. The reaction mixture was cooled, concentrated in vacuo and purified by chromatography (silica gel, 0 to 5% [9:1 methanol/20M ammonia] in dichloromethane to give the title compound (0.56 g) as a beige solid; ESMS m/z 427.2 [M+H]⁺.

### g) 9-(3-tert-Butoxycarbonylamino-propyl)-3-methyl-7-oxo-1H,7H-[1,3]oxazino[5,4,3-ij]quinoline-6-carboxylic acid ethyl ester

A solution of Intermediate 1f (0.507 g, 1.19 mmol) in dichloromethane (30 mL) and was treated with 10% palladium on carbon (0.20 g) and hydrogenated at room temperature and atmospheric pressure for 17 h. The reaction mixture was filtered and concentrated to give the title compound (0.53 g) as an yellow solid; ESMS m/z 431.2 [M+H]⁺.

### h) 9-(3-tert-Butoxycarbonylamino-propyl)-3-methyl-7-oxo-1H,7H-[1,3]oxazino[5,4,3-ij]quinoline-6-carboxylic acid sodium salt

A solution of Intermediate 1 g (0.51 g, 1.19 mmol) in tetrahydrofuran (10 mL) was treated with 2M sodium hydroxide (0.65 mL). After stirring at 50° C. for 17 h the mixture was treated with solid carbon dioxide and concentrated to give the title compound as a pale yellow solid (0.486 g); ESMS m/z 403.1 [M+H]⁺.

### i) 9-(3-aminopropyl)-3-methyl-7-oxo-1H,7H-[1,3]oxazino[5,4,3-ij]quinoline-6-carboxylic acid trifluoroacetate salt

A solution of Intermediate 1 h (0.486 g, 1.19 mmol) in dichloromethane (1 mL) was treated with trifluoroacetic acid (1 mL) and the reaction stirred at room temperature for 0.25 h. The solution was evaporated to dryness to give the title compound (0.50 g) as a white solid; ESMS m/z 303.1 [M+H]⁺.

### Intermediate 2: 2-(3-Aminopropyl)-4-oxo-8,9-dihydro-4H,7H-10-oxa-6a-aza-cyclohepta[de]naphthalene-5-carboxylic trifluoroacetate salt

### a) 8-Fluoro-1-(3-hydroxypropyl)-6-iodo-4-oxo-1,4-dihydro-quinoline-3-carboxylic acid ethyl ester

A suspension of 8-fluoro-6-iodo-4-oxo-1,4-dihydro-quinoline-3-carboxylic acid ethyl ester (J. Tucker et al., WO 99/32450) (0.722 g, 2 mmol), sodium iodide (0.3 g, 2 mmol), sodium carbonate (0.636 g, 6 mmol) and 3-bromo propanol (1.8 mL, 20 mmol) in dimethylformamide (5 mL) was heated at 80° C. for 3 h. The reaction mixture was cooled, filtrated and concentrated in vacuo. The residue was preabsorbed on silica gel and purified by chromatography (silica gel, 0 to 1% [9:1 methanol/20M ammonia] in dichloromethane) to give the title compound as a beige solid (0.62 g); ESMS m/z 420.1 [M+H]⁺.

### b) 2-Iodo-4-oxo-8,9-dihydro-4H,7H-10-oxa-6a-aza-cyclohepta[de]naphthalene-5-carboxylic acid ethyl ester

A solution of Intermediate 2a (0.575 g, 1.37 mmol) and 1,8-diazabicyclo[5.4.0]undec-7-ene (0.205 mL, 1.37 mmol) in dimethylformamide (14 mL) was heated at 100° C. for 17 h. The reaction mixture was concentrated in vacuo and the residue was purified by chromatography (silica gel, 0 to 5% [9:1 methanol/20 M ammonia] in dichloromethane) to give the title compound as a pale yellow solid (0.504 g); ESMS m/z 400.1 [M+H]⁺.

### c) 2-(3-tert-Butoxycarbonylamino-prop-1-ynyl)-4-oxo-8,9-dihydro-4H,7H-10-oxa-6a-aza-cyclohepta[de]naphthalene-5-carboxylic acid ethyl ester

A mixture of Intermediate 2b (0.493 g, 1.23 mmol), copper (I) iodide (0.026 g, 0.13 mmol) and triethylamine (6 mL, 43 mmol) were suspended in dry acetonitrile (12 mL). The suspension was heated to 50° C. whilst argon was bubbled through. After 20 min, dichlorobis(triphenylphosphine)palladium (II) (0.026 g, 0.37 mmol) and N-tert-butoxycarbonylpropargylamine (0.326 g, 2.09 mmol) were added and the mixture was heated at 50° C. for 1.5 h. The reaction mixture was cooled, concentrated in vacuo and purified by chromatography (silica gel, 0 to 5% [9:1 methanol/20M ammonia] in dichloromethane) to give the title compound (0.7 g) as a beige solid; ESMS m/z 427.3 [M+H]⁺.

### d) 2-(3-tert-Butoxycarbonylamino-propyl)-4-oxo-8,9-dihydro-4H,7H-10-oxa-6a-aza-cyclohepta[de]naphthalene-5-carboxylic acid ethyl ester

A solution of Intermediate 2c (0.7 g, 1.23 mmol) in dichloromethane (150 mL) was treated with 10% palladium on carbon (0.3 g) and hydrogenated at room temperature and atmospheric pressure for 17 h. The reaction mixture was filtered, concentrated and purified by chromatography (silica gel, 0 to 5% [9:1 methanol/20M ammonia] in dichloromethane) to give the title compound (0.5 g) as a pale yellow solid; ESMS m/z 431.4 [M+H]⁺.

### e) 2-(3-tert-Butoxycarbonylamino-propyl)-4-oxo-8,9-dihydro-4H,7H-10-oxa-6a-aza-cyclohepta[de]naphthalene-5-carboxylic acid sodium salt

A solution of Intermediate 2d (0.5 g, 1.16 mmol) in tetrahydrofuran (10 mL) and 1,4-dioxan (12 mL) was treated with 2M sodium hydroxide (0.96 mL). The reaction was stirred for 17 h at 50° C. then at 60° C. for 7 h. Solid carbon dioxide was added and the reaction mixture was concentrated to give the title compound (0.660 g) as a pale yellow solid; ESMS m/z 403.3 [M+H]⁺.

### f) 2-(3-Amino-propyl)-4-oxo-8,9-dihydro-4H,7H-10-oxa-6a-aza-cyclohepta[de]naphthalene-5-carboxylic acid trifluoroacetate salt

A solution of Intermediate 2e (0.49 g, 1.16 mmol) in dichloromethane (10 mL) was treated with trifluoroacetic acid (3 mL) and the reaction stirred at room temperature for 20 min. The solution was evaporated to give the title compound (0.5 g) as a beige solid; ESMS m/z 303.3 [M+H]⁺.

### Intermediate 3: 9-(3-Aminopropyl)-3,3-dimethyl-7-oxo-1H,7H-[1,3]oxazino[5,4,3-ij]quinoline-6-carboxylic acid trifluoroacetate salt

### a) 9-Iodo-3,3-dimethyl-7-oxo-1H,7H-[1,3]oxazino[5,4,3-ij]quinoline-6 carboxylic acid ethyl ester

A suspension of Intermediate 1d (3.45 g, 9.24 mmol), p-toluene sulfonic acid (1.45 g, 7.6 mmol) and dimethoxypropane (11.3 mL, 91.7 mmol) in N-methylpyrrolidinone (8 mL) was heated at 80° C. for 3 h. The reaction mixture was concentrated and the residue partitioned between water and dichloromethane. The organic extracts were combined, dried (MgSO₄), filtered, and concentrated in vacuo to give the title compound as a mixture of the ethyl and methyl esters; (3.18 g); ESMS m/z 414.0 and 400.0 [M+H]⁺.

### b) 9-(3-tert-Butoxycarbonylamino-prop-1-ynyl)-3,3-dimethyl-7-oxo-1H,7H-[1,3]oxazino[5,4,3-ij]quinoline-6-carboxylic acid ethyl ester

A mixture of Intermediate 3a (3.1 g, 7.66 mmol), copper (I) iodide (0.166 g, 0.87 mmol) and triethylamine (38 mL, 273 mmol) were suspended in dry acetonitrile (75 mL). The suspension was heated to 50° C. whilst argon was bubbled through. After 20 min, dichlorobis(triphenylphosphine)palladium (II) (0.166 g, 0.238 mmol) and N-tert-butoxycarbonylpropargylamine (2 g, 13 mmol) were added and the mixture was heated at 50° C. for 1.5 h. The reaction mixture was cooled, concentrated in vacuo and purified by chromatography (silica gel, 0 to 5% [9:1 methanol/20 M ammonia] in dichloromethane) to give the title compound (3 g) as a mixture of the ethyl and methyl esters; ESMS m/z 441.3 and 427.3 [M+H]⁺.

### c) 9-(3-tert-Butoxycarbonylaminopropyl)-3,3-dimethyl-7-oxo-1H,7H-[1,3]oxazino[5,4,3-ij]quinoline-6-carboxylic acid ethyl ester

A solution of Intermediate 3b (3 g, 7.2 mmol) in dichloromethane (100 mL) and methanol (100 mL) was treated with 10% palladium on carbon (1 g) and hydrogenated at room temperature and atmospheric pressure for 65 h. The reaction mixture was filtered and concentrated to give the title compound (1.3 g) as a mixture of the ethyl and methyl esters; ESMS m/z 445.4 and 431.3 [M+H]⁺.

### d) 9-(3-tert-Butoxycarbonylaminopropyl)-3,3-dimethyl-7-oxo-1H,7H-[1,3]oxazino[5,4,3-ij]quinoline-6-carboxylic acid sodium salt

A solution of Intermediate 3c (1.3 g, 3 mmol) in tetrahydrofuran (30 mL) was treated with 2M sodium hydroxide (1.7 mL). The reaction was stirred for 17 h at 50° C. then solid carbon dioxide was added and the reaction mixture was concentrated to give the title compound (1.4 g) as a white solid; ESMS m/z 417.3[M+H]⁺.

### e) 9-(3-Aminopropyl)-3,3-dimethyl-7-oxo-1H,7H-[1,3]oxazino[5,4,3-ij]quinoline-6-carboxylic acid trifluoroacetate salt

A solution of Intermediate 3d (1.4 g, 3 mmol) in dichloromethane (50 mL) was treated with trifluoroacetic acid (15 mL) and the reaction stirred at room temperature for 20 min. The solution was evaporated to dryness then purified by preparative reverse phase HPLC to give the title compound (1.13 g) as a beige solid; ESMS m/z 317.3 [M+H]⁺.

### Intermediate 4: 9-(3-Aminopropyl)-7-oxo-1H,7H-[1,3]oxazino[5,4,3-ij]quinoline-6-carboxylic acid trifluoroacetate salt

### a) Ethyl 9-iodo-7-oxo-1H,7H-[1,3]oxazino[5,4,3-ij]quinoline-6-carboxylate

A suspension of Intermediate 1d (0.44 g, 1.18 mmol) and potassium carbonate 0.488 g, 3.54 mmol) in DMF (8 mL) was treated with chloroiodomethane (0.8 mL, 11 mmol) and the resultant mixture heated at 100° C. After 17 h the reaction was cooled and evaporated to yield the crude product. Chromatography over silica gel eluting with 0-5% methanol in dichloromethane gave the title compound (0.30 g) as a pale green solid; ESMS m/z 386.0 [M+H]⁺.

### b) Ethyl 9-[3-({[(1,1-dimethylethyl)oxy]carbonyl}amino)-1-propyn-1-yl]-7-oxo-1H,7H-[1,3]oxazino[5,4,3-ij]quinoline-6-carboxylate

A mixture of Intermediate 4a (0.297 g, 0.77 mmol), copper (I) iodide (0.016 g, 0.087 mmol) and triethylamine (3.75 mL, 27 mmol) were suspended in dry acetonitrile (8 mL). The suspension was heated to 50° C. whilst argon was bubbled through. After 20 min, dichlorobis(triphenylphosphine)palladium (II) (0.016 g, 0.024 mmol) and N-tert-butoxycarbonylpropargylamine (0.203 g, 1.3 mmol) were added and the mixture was heated at 50° C. for 1.5 h. The reaction mixture was cooled, concentrated in vacuo and purified by chromatography (silica gel, 0 to 5% methanol in dichloromethane to give the title compound (0.23 g); ESMS m/z 413.2 [M+H]⁺.

### c) Ethyl 9-[3-({[(1,1-dimethylethyl)oxy]carbonyl}amino)propyl]-7-oxo-1H,7H-[1,3]oxazino[5,4,3-ij]quinoline-6-carboxylate

A solution of Intermediate 4b (0.225 g, 0.545 mmol) in dichloromethane (10 mL) and methanol (10 mL) was treated with 10% palladium on carbon (0.1 g) and hydrogenated at room temperature and atmospheric pressure for 16 h. The reaction mixture was filtered and concentrated to give the title compound (0.21 g); ESMS m/z 417.2 [M+H]⁺.

### d) 9-[3-({[(1,1-Dimethylethyl)oxy]carbonyl}amino)propyl]-7-oxo-1H,7H-[1,3]oxazino[5,4,3-ij]quinoline-6-carboxylic acid sodium salt

A solution of Intermediate 4c (0.205 g, 0.5 mmol) in tetrahydrofuran (5 mL) was treated with 2M sodium hydroxide (0.40 mL). The reaction was stirred for 17 h at 50° C. then solid carbon dioxide was added and the reaction mixture was concentrated to give the title compound (0.21 g) as a pale yellow solid; ESMS m/z 389.1 [M+H]⁺.

### e) 9-(3-Aminopropyl)-7-oxo-1H,7H-[1,3]oxazino[5,4,3-ij]quinoline-6-carboxylic acid trifluoroacetate salt

A solution of Intermediate 4d (0.20 g, 0.5 mmol) in dichloromethane (3 mL) was treated with trifluoroacetic acid (3 mL) and the reaction stirred at room temperature for 20 min. The solution was evaporated to dryness to give the title compound (0.20 g); ESMS m/z 289.1 [M+H]⁺.

### Intermediate 5: 9-(3-Aminopropyl)-3,3-dimethyl-7-oxo-2,3-dihydro-7H-[1,4]oxazino[2,3,4-ij]quinoline-6-carboxylic acid

### a) Ethyl (2Z)-3-(dimethylamino)-2-[(2,3,5-trifluorophenyl)carbonyl]-2-propenoate

A suspension of 2,3-difluoro-5-iodobenzoic acid (2.84 g, 10 mmol) in dichloromethane (50 mL) was treated with oxalyl chloride (1.3 mL, 14.9 mmol). After 1.5 h the resultant solution was evaporated, re-dissolved in toluene (50 mL) and re-evaporated to yield the intermediate acid chloride. This crude material was dissolved in toluene (50 mL) and treated with triethylamine (2.1 mL, 15 mmol) and ethyl 3-dimethylaminopropeneoate (1.86 g, 12.9 mmol). The mixture was stirred for 2 h at 90° C., cooled, filtered and evaporated. The crude product was purified by chromatography over silica gel eluting with 0-70% ethyl acetate in hexane to give the title compound (3.05 g); ESMS m/z 410.2 [M+H]⁺.

### b) Ethyl (2Z)-3-[(2-hydroxy-1,1-dimethylethyl)amino]-2-[(2,3,5-trifluorophenyl)carbonyl]-2-propenoate

Intermediate 5a (0.408 g, 1 mmol) was dissolved in ethanol (5 mL) and 2,2-dimethyl-2-aminoethanol (0.098 g, 1.1 mmol). After stirring for 1.25 h the mixture was evaporated and the crude product purified by chromatography over silica gel eluting with 0-40% ethyl acetate in dichloromethane to give the title compound (0.45 g); ESMS m/z 454.2 [M+H]⁺.

### c) Ethyl 8-iodo-3,3-dimethyl-6-oxo-2,3,3a,6-tetrahydrobenzo[de]chromene-5-carboxylate

Intermediate 5b (0.405 g, 0.89 mmol) was dissolved in DMF (9 mL) and treated with diazabicycloundecane (0.27 mL, 1.78 mmol). After heating at 60° C. for 16 h the mixture was cooled and the DMF evaporated and the crude product purified by chromatography over silica gel eluting with 0-40% ethyl acetate in dichloromethane to give the title compound (0.34 g); ESMS m/z 414.2 [M+H]⁺.

### d) Ethyl 8-[3-({[(1,1-dimethylethyl)oxy]carbonyl}amino)-1-propyn-1-yl]-3,3-dimethyl-6-oxo-2,3,3a,6-tetrahydrobenzo[de]chromene-5-carboxylate

Through a mixture of Intermediate 5c (0.337 g, 0.816 mmol) and copper (I) iodide (0.017 g, 0.086 mmol) suspended in acetonitrile (8 mL) at 50° C. was bubbled argon. After 20 min dichlorobis(triphenylphosphine)palladium (II) (0.017 g, 0.024 mmol) and N-tert-butoxycarbonylpropargylamine (0.216 g, 1.38 mmol) were added and heating continued for a further 1 h. The mixture was cooled, the acetonitrile evaporated and the crude product purified by chromatography over silica gel eluting with 0-2% methanol in dichloromethane to give the title compound (0.35 g); ESMS m/z 441.4 [M+H]⁺.

### e) Ethyl 8-[3-({[(1,1-dimethylethyl)oxy]carbonyl}amino)propyl]-3,3-dimethyl-6-oxo-2,3,3a,6-tetrahydrobenzo[de]chromene-5-carboxylate

A solution of Intermediate 5d (0.35 g, 0.795 mmol) in dichloromethane (20 mL) was treated with 10% palladium on carbon (0.35 g) and hydrogenated at room temperature and atmospheric pressure for 16 h. The reaction mixture was filtered and concentrated to give the title compound (0.34 g); ESMS m/z 445.4 [M+H]⁺.

### f) 8-[3-({[(1,1-Dimethylethyl)oxy]carbonyl}amino)-1-propyl]-3,3-dimethyl-6-oxo-2,3,3a,6-tetrahydrobenzo[de]chromene-5-carboxylic acid sodium salt

A solution of Intermediate 5e (0.34 g, 0.76 mmol) in tetrahydrofuran (5 mL) was treated with 2M sodium hydroxide (0.42 mL). The reaction was stirred for 17 h at 50° C. then solid carbon dioxide was added and the reaction mixture was concentrated to give the title compound (0.32 g); ESMS m/z 417.3 [M+H]⁺.

### g) 8-(3-Aminopropyl)-3,3-dimethyl-6-oxo-2,3,3a,6-tetrahydrobenzo[de]chromene-5-carboxylic acid trifluoroacetate salt

A solution of Intermediate 5f (0.316 g, 0.76 mmol) in dichloromethane (3 mL) was treated with trifluoroacetic acid (3 mL) and the reaction stirred at room temperature for 20 min. The solution was evaporated to dryness to yield the title compound (0.24 g) as a beige solid; ESMS m/z 317.3 [M+H]⁺.

### Intermediate 6: 4″-O-Propenoylerythromycin A (9E)-oxime

### a) 2′-O-Acetylerythromycin A (9E)-acetyloxime

A solution of erythromycin A (9E)-oxime (8.5 g, 1.3) in dichloromethane (130 mL) was treated with sodium bicarbonate (2.09 g) followed by acetic anhydride (2.35 mL). After stirring overnight at room temperature the mixture was diluted with dichloromethane and washed with water. The organic layer was separated, dried and evaporated. The crude product was taken up in ethyl acetate and rewashed with saturated aqueous sodium bicarbonate. The organic layer was separated, dried and evaporated to yield the title compound as a solid; ESMS m/z 833.9 [M+H⁺].

### b) 2′-O-Acetyl-4″-O-propenoyl erythromycin A (9E)-acetyloxime

A mixture of Intermediate 6a (8 g), triethylamine (4 mL) and 3-chloropropionyl chloride (1.37 mL) in toluene (200 mL) was stirred at 20° C. for 20 h. The reaction mixture was concentrated by evaporation under reduced pressure then partitioned between a saturated solution of NH₄Cl and ethyl acetate. The organic phase was dried over Na₂SO₄, filtered and evaporated under reduced pressure. The crude product was purified by flash-chromatography 0-10% (9:1 MeOH/20 M NH₃) in dichloromethane affording the title compound (4.0 g); ESMS m/z 887.6 [M+H]⁺.

### c) 4″-O-Propenoylerythromycin A (9E)-oxime

Intermediate 6b (4.0 g) was dissolved in MeOH (200 mL) and stirred at 55° C. for 20 h, then at room temperature for 72 h. The solvent was evaporated under reduced pressure affording the title compound (3.53 g); ESMS m/z 803.5 [M+H]⁺.

### Intermediate 7: 4″-O-Propenoyl erythromycin A (9E)-methoxime

### a) 2′-O-Acetylerythromycin A (9E)-methoxime

A solution of erythromycin A (9E) methoxime (5.7 g) in dichloromethane (70 mL) was treated with triethylamine (2.25 mL) followed by acetic anhydride (1.18 mL). After stirring overnight at room temperature the mixture was diluted with dichloromethane and washed with aqueous sodium bicarbonate. The organic layer was separated, dried and evaporated to yield the title compound as a solid; ESMS m/z 805.8 [M+H⁺].

### b) 2′-O-Acetyl-4″-O-propenoyl erythromycin A (9E)-methoxime

Using a similar procedure to that described in Intermediate 6b, Intermediate 7a (5.3 g) gave the title compound as a white solid; ESMS m/z 859.8 [M+H⁺].

### c) 4″-O-Propenoyl erythromycin A (9E)-methoxime

Using a similar procedure to that described in Intermediate 6c, Intermediate 7b (4.17 g) gave the title compound as a white solid; ESMS m/z 817.6 [M+H⁺].

### Intermediate 8: 4″-O-Propenoyl-6-O-methylerythromycin A

### a) 2′-O-Acetyl-4″-O-propenoyl-6-O-methyl-erythromycin A

To a solution of 2′-O-acetyl-6-O-methyl-erythromycin A (1.1 g) in dichloromethane (20 mL) pyridine (1.7 mL) and acryloyl chloride (1.1 mL) were added at 0° C. After 2 h a further addition of pyridine (1.7 mL) and of acryloyl chloride (1.1 mL) was performed. The reaction mixture was quenched with a saturated solution of NH₄Cl (10 mL) and extracted with dichloromethane (3×20 mL). The organic phase was washed with a saturated solution of NaHCO₃ (10 mL), water (10 mL), dried (Na₂SO₄), filtered and evaporated under reduced pressure. The crude product was purified by flash-chromatography, 0-10% (9:1 MeOH/20M NH₃) in dichloromethane, affording the title compound (470 mg); ESMS m/z 844 [M+H]⁺.

### b) 4″-O-Propenoyl-6-O-methyl-erythromycin A

Intermediate 8a (1.82 g) was dissolved in MeOH (100 mL) and stirred at 60° C. for 4 h, then at room temperature for 16 h. The solvent was evaporated under reduced pressure and the crude product was purified by flash chromatography (eluent: MeOH/DCM/NH₄OH 5/90/0) affording the title compound; ESMS m/z 802 [M+H]⁺.

### Intermediate 9: 4″-O-Propenoyl-azithromycin-11,12-carbonate

### a) 2′-O-Acetyl-4″-O-propenoyl-azithromycin-11,12-carbonate

A solution of 2′-O-acetyl-azithromycin-11,12-carbonate (10.9 g) in toluene (300 mL) was stirred at room temperature under argon atmosphere. To this solution triethylamine (12.66 mL) and 3-chloro-propionyl chloride (1.94 mL) were added in two portions over a period of 10 minutes. After 20 minutes the solution was diluted with a saturated aqueous solution of NaHCO₃ (300 mL) and extracted with toluene (4×80 mL). The collected organic phase was dried, filtered and concentrated under reduced pressure affording the title compound (11.0 g); ESMS m/z 872 [M+H]⁺.

### b) 4″-O-Propenoyl-azithromycin-11,12-carbonate

A solution of Intermediate 9a (11.0 g) in MeOH (200 mL) was stirred at room temperature for 48 h. The solvent was evaporated under reduced pressure affording the title compound (9.81 g); ESMS m/z 872 [M+H]⁺.

### Intermediate 10: 4″-O-Propenoyl-azithromycin

To a solution of Intermediate 9a (1.3 g, 1.49 mmol) in acetonitrile (50 mL), a saturated aqueous solution of potassium carbonate (30 mL) was added at room temperature. The resulting mixture was heated to 70° C. for 8 h. The mixture was then diluted with water (100 mL), extracted with ethyl acetate (4×30 mL). The combined organic phases were dried, filtered and evaporated. The crude product was purified by chromatography over silica eluting with 0-10% (9:1 MeOH/20M NH₃) in dichlormethane to give the title compound as a white solid; ESMS m/z 872 [M+H]⁺.

### Intermediate 11: 4″-O-Propenoylerythromycin A (9E)-methoxymethyloxime

### a) Erythromycin A (9E)-methoxymethyloxime

A solution of erythromycin A (9E)-oxime (10.11 g, 13.5 mmol) dissolved in dichloromethane (25 mL) at 5° C. was treated with tetra-n-butylammonium bromide (0.486 g) and 2M sodium hydroxide solution (25 mL). The resultant mixture was stirred vigorously and chloromethoxymethane (1.5 mL, 20 mmol), added in a single portion. After 0.75 h the mixture was quenched by the addition of brine (50 mL) and the organic material extracted with chloroform (2×150 mL). The combined organic phases were dried and evaporated to yield the crude product. Crystallisation from ethyl acetate gave the title compound as white solid; ESMS m/z 793.8 [M+H]⁺.

### b) 2′-O-Acetylerythromycin A (9E)-methoxymethyloxime

A suspension of Intermediate 11a (3.94 g, 4.96 mmol) in dichloromethane (20 mL) was treated with acetic anhydride (0.57 g, 5.58 mmol). After stirring for 48 h water (30 mL) was added and the pH of the mixture adjusted to 9-10 by the addition of 2M sodium hydroxide solution. The organic material was extracted with dichloromethane (2×50 mL) and the combined extracts were dried and evaporated to yield the title compound as a white solid; ESMS m/z 835.8 [M+H]⁺.

### c) 2′-O-Acetyl-4″-O-propenylerythromycin A (9E)-methoxymethyloxime

A solution of Intermediate 11b (3.8 g, 4.55 mmol) dissolved in toluene (50 mL) and triethylamine was treated with 3-chloropropionyl chloride (0.54 mL, 5.7 mmol). After stirring for 24 h the mixture was diluted with ethyl acetate (100 mL) and washed with water (2×50 mL). The organic phase was separated, dried and evaporated to give the crude product. Purification by chromatography over silica gel eluting with 0-10% (9:1 MeOH/20 M NH₃) in dichlormethane to give the title compound as a white solid; ESMS m/z 889.9 [M+H]⁺.

### d) 4″-O-Propenylerythromycin A (9E)-methoxymethyloxime

A solution of Intermediate 11c (1.80 g, 2.02 mmol) dissolved in methanol (50 mL) was stirred at 50° C. for 16 h. The methanol was evaporated to yield the title compound (83%) as a white solid; ESMS m/z 877.7 [M+H]⁺.

### Intermediate 12 4″-O-Allyl-6-O-methyl-erythromycin A

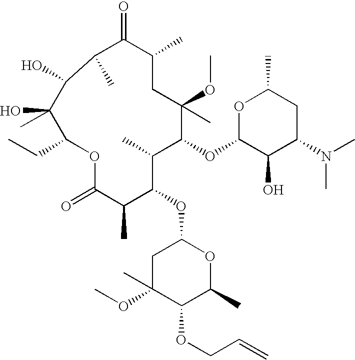

### a) 4″-O-(1-Imidazol-1-yl-carbonyl)-6-O-methyl-erythromycin A

6-O-Methyl-erythromcyin A (108 g, 0.144 mol) in dry THF (500 mL) under an atmosphere of argon was treated portionwise with carbonyldiimidazole (43.2 g, 0.267 mol) with ice bath cooling. After 1 h the cooling bath was removed. After 24 h additional THF (300 mL) was added to dissolve any solid material formed followed by the dropwise addition of water (500 mL). After stirring for 2.5 h a thick white precipitate had formed. The mixture was filtered under vacuum, washed with cold water (2×250 mL) and dried under vacuum to yield the title compound (100 g) as a white solid. The mother liquors yielded further title compound (12 g) after standing overnight; ESMS m/z 842.7 [M+H]⁺.

### b) 4″-O-(Allyloxycarbonyl)-6-O-methyl-erythromycin A

Intermediate 12a (50 g, 59.3 mmol) in DCM (200 mL) was cooled to 0° C. and treated with allyl alcohol (23.8 mL) and DBU (9.1 mL, 61 mmol). The reaction was stirred at 0° C. for 2.5 h and at 20° C. for 1.75 h. The reaction mixture was quenched with 3% aqueous citric acid (100 mL), the phases separated, and the organic phase washed with saturated sodium hydrogen carbonate and brine. After drying and evaporation to dryness, the residue was triturated with petroleum ether (bp 40-60° C.) to give the title compound as a white solid (41 g); ESMS m/z 832.6 [M+H]⁺.

### c) 4″-O-(Allyloxycarbonyl)-9-dihydro-9-methoxy-2′,11-bis-O-trimethylsilyl-9,12-anhydro-6-O-methyl-erythromycin A

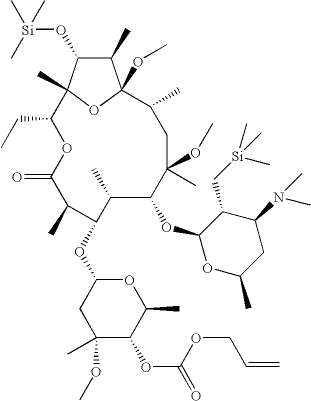

Intermediate 12b (50 g, 0.06 mol) in dry pyridine (150 mL) under argon was treated dropwise with chlorotrimethylsilane (36.3 mL). After 4.5 h at room temperature the reaction mixture was evaporated to dryness under reduced pressure and the residue taken up in toluene (300 mL) and re-evaporated. This process was repeated a further one time. The resultant solid thus obtained was dissolved in methanol (250 mL) and stirred for 10 h at room temperature. The methanol was evaporated and the residue partitioned between diethyl ether (400 mL) and saturated sodium bicarbonate solution (500 mL). The organic layer was separated and the aqueous phase extracted with fresh diethyl ether (2×200 mL) The combined organic phases were washed with water, dried and evaporated to give a white foam (57 g). To a solution of this material in dry pyridine (150 mL) was added chlorotrimethylsilane (25.0 mL). After 2 h the pyridine was evaporated and the resultant solid partitioned between diethyl ether and saturated sodium bicarbonate solution (sufficient aqueous was used to ensure a terminal pH=9). The layers were separated and the aqueous phase was extracted with diethyl ether (2×200 mL). The combined organics were washed with water (400 mL) and brine (150 mL). Drying over sodium sulfate and evaporation yielded a syrup which was dissolved in toluene and re-evaporated. After drying under high vacuum overnight a white solid was obtained (60 g). Crystallisation from acetonitrile (250 mL) gave 38.2 g of a white solid; ESMS m/z 990.7 [M+H]⁺.

### d) 4″-O-Allyl-6-O-methyl-erythromycin A

The Intermediate 12c (15.0 g, 15.2 mmol) in dry THF (100 mL) under argon was treated with tetrakis(triphenylphosphine)palladium (0.36 g) and the resultant mixture heated at reflux for 1.5 h. Monitoring by lc/ms indicates a 3:2 mixture of the desired product and the 4″-hydroxy derivative. Allyl t-butyl carbonate (5 mL) (F. Houlihan et al, Can. J. Chem. 1985, 63, 153) was added and heating continued for a further 3.75 h. After cooling and standing overnight at 20° C. the THF was evaporated and the dark brown residue taken up in 40/60 petroleum ether (100 mL). The solution was treated with charcoal, filtered and evaporated. The solid was then taken up in acetonitrile and re-evaporated and dried under vacuum overnight to yield 15.89 g. The product (12.8 g) was dissolved in acetonitrile (25 mL) and 10% aqueous acetic acid (130 mL). After stirring at 20° C. for 6 h diethyl ether (50 mL) was added and the layers separated, the organic layer was extracted with water and the combined aqueous extracts made basic by the addition of potassium carbonate. The organic product was extracted with EtOAc (2×100 mL), dried and evaporated to give the title compound as a solid (10.0 g); ESMS m/z 946.7 [M+H]⁺.

### Intermediate 13 4″-O-(2-oxoethyl)-6-O-methyl-erythromycin A

Intermediate 12 (95.8 g, 121 mmol) in DCM (1 L) and methanol was cooled to −78° C. and TFA (18 mL) added. Ozonized oxygen was bubbled through until a blue colour developed (1.25 h). Argon was bubbled through the mixture to flush out the ozone, then dimethyl sulfide (35 mL) and triethylamine (50.4 mL) were added. The reaction was stirred at −78° C. for 30 min then removed from the cooling bath. After 0.5 h the reaction was warmed to 0° C. in a water bath and stirred for a further 0.5 h. The reaction mixture was washed with water (500 mL), dried (Na₂SO₄) and evaporated to dryness. The residue was dissolved in toluene and evaporated three times to give the title compound (103.7 g) which was used without purification; ES m/z 822.7 [M+MeOH+H]⁺, 834.6 [M+HCO₂]⁻.

### Intermediate 14: 9-[(2-Aminoethyl)thio]-7-oxo-2,3-dihydro-7H-[1,4]oxazino[2,3,4-ij]quinoline-6-carboxylic acid

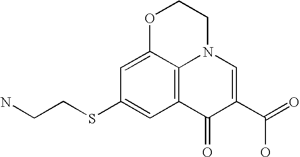

### a) 3-(Dimethylamino)-2-[(2,3,5-trifluorophenyl)carbonyl]-2-propenoic acid ethyl ester

DMF (20 drops) and oxalyl chloride (15 mL, 171 mmol) were added to a solution of 2,3,5-trifluorobenzoic acid (20.12 g, 114 mmol) in dichloromethane (500 mL) at 0° C. under argon. After 15 minutes the reaction was removed from the ice bath and stirring continued at 20° C. until gas evolution had stopped. The mixture was concentrated and the residue mixed with toluene and reconcentrated. The residue was dissolved in toluene (500 mL), and triethylamine (23.9 mL, 171 mmol) and treated with ethyl 3-(dimethylamino)-2-propenoate (21.27 g, 149 mmol). After heating the resultant mixture at 90° C. under argon for 3 hours the reaction was concentrated and the residue purified by chromatography (silica gel, 50 to 80% diethylether in hexane) to give the title compound (6.02 g); ESMS m/z 324.2 [M+Na]⁺.

### b) 3-[(2-Hydroxyethyl)amino]-2-[(2,3,5-trifluorophenyl)carbonyl]-2-propenoic acid ethyl ester

2-Aminoethanol (0.68 mL, 11.2 mmol) in ethanol (5 mL) was added to a stirred solution of Intermediate 14a (3.08 g, 10.2 mmol) in ethanol (35 mL) at 20° C. and stirred for 16 hours. The mixture was concentrated and the residue purified by chromatography (silica gel, 0 to 4% methanol in dichloromethane) to give the title compound (2.74 g); ESMS m/z 318.4 [M+H]⁺.

### c) 9-Fluoro-7-oxo-2,3-dihydro-7H-[1,4]oxazino[2,3,4-ij]quinoline-6-carboxylic acid ethyl ester

A mixture of Intermediate 14b (2.70 g, 8.51 mmol) and DBU (1.3 mL, 8.51 mmol) in DMF (40 mL) was stirred at 70° C. for 2 days. The mixture was concentrated and the yellow residue washed with dichloromethane. The residual solid was collected and dried to give the title compound (2.06 g); ESMS m/z 278.3 [M+H]⁺.

### d) 9-{[2-({([(1,1-Dimethylethyl)oxy]carbonyl}amino)ethyl]thio}-7-oxo-2,3-dihydro-7H-[1,4]oxazino[2,3,4-ij]quinoline-6-carboxylic acid ethyl ester

A mixture of Intermediate 14c (2.04 g, 7.35 mmol), 1,1-dimethylethyl (2-mercaptoethyl)carbamate (5 mL, 29.4 mmol) and potassium carbonate (4.06 g, 29.4 mmol) in DMSO (20 mL) was stirred at 100° C. for 5 hours. The mixture was concentrated and the residue partitioned between dichloromethane and water. The organic layer was dried, concentrated, and the residue purified by chromatography (silica gel, 0 to 4% methanol in dichloromethane) to give the title compound (2.00 g); ESMS m/z 435.3 [M+H]⁺.

### e) 9-[(2-Aminoethyl)thio]-7-oxo-2,3-dihydro-7H-[1,4]oxazino[2,3,4-ij]quinoline-6-carboxylic acid

A mixture of Intermediate 14d (1.89 g, 4.35 mmol), 2M hydrochloric acid (1.5 mL) and dioxan (1.5 mL) were stirred together for 16 hours. The mixture was concentrated and the residue triturated with dioxan to give the title compound (2.00 g) as a yellow solid; ESMS m/z 307.3 [M+H]⁺.

### Intermediate 15: 10-(3-Aminopropyl)-8-oxo-3,4-dihydro-1H,8H-[1,4]oxazepino[6,5,4-ij]quinoline-7-carboxylic acid

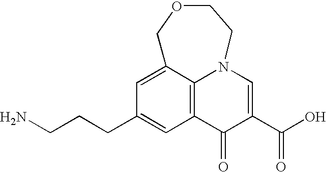

### a) (2-Amino-5-bromophenyl)methanol

1.0M Lithium aluminium hydride in THF (175 mL, 175 mmol) was added to a solution of 2-amino-5-bromobenzoic acid methyl ester (40.37 g, 175 mmol) in THF (400 mL) by cannula at 0° C. under argon. Stirring was continued for 1 hour after addition. Water (6.4 mL), 2M sodium hydroxide (6.4 mL) and water (12.8 mL) were added sequentially and the resultant mixture stirred at 20° C. for 30 minutes before filtration through celite. The solution was evaporated and the residue crystallised from ethyl acetate/hexane to give the title compound as a white solid (18.50 g); ESMS m/z 202.1 [M+H]⁺.

### b) N-[4-Bromo-2-(hydroxymethyl)phenyl]-2-chloroacetamide

To a suspension of Intermediate 15a (2.48 g, 12.28 mmol) in dry diethyl ether (70 mL) at 0° C. was added dropwise a solution of chloroacetyl chloride (1.39 g) in dry toluene (25 mL). To the white suspension was added a solution of triethylamine (5.1 mL) in dry toluene (25 mL). After 1 h the reaction mixture was treated with water and chloroform. The organic phase was dried, filtered, and concentrated in vacuo to give a brown oil (4.45 g) containing the title compound; ESMS m/z 278.0/280.0 [M+H]⁺ and the bis-acetylated compound {5-bromo-2-[(chloroacetyl)amino]phenyl}methyl chloroacetate; ESMS m/z 352.0/354.0/356.0 [M+H]⁺.

### c) 7-Bromo-1,5-dihydro-4,1-benzoxazepin-2(3M-one

A fresh solution of sodium ethoxide (22 mmol) in ethanol was prepared from sodium (500 mg) and dry ethanol (60 mL). This solution was added to Intermediate 15b (4.25 g, containing a mixture of mono N-acetylated (6 mmol) and bis-acetylated (7.3 mmol) material). The suspension was heated to 80° C. for 1.5 h then allowed to cool down. Filtration of the solid followed by trituration with hexane provided the title compound (3 g) as a beige solid; ESMS m/z 242.11244.1 [M+H]⁺.

### d) 7-Bromo-1,2,3,5-tetrahydro-4,1-benzoxazepine

To a suspension of Intermediate 15c (2.5 g, 10.3 mmol)) in dry diethyl ether (60 mL) was added a solution of lithium aluminium hydride in THF (1M, 2.58 mL). After 1.25 h an additional amount of lithium aluminium hydride in THF (1M, 10 mL) was added to the reaction mixture and stirred for one hour. After the addition of water (6 mL), the mixture was filtered and the solid washed with diethyl ether. The filtrate was evaporated and re-dissolved in water. Freeze-drying of this aqueous solution gave the title compound (2 g) as a white solid; ESMS m/z 228.1/230.1 [M+H]⁺.

### e) [(7-Bromo-2,3-dihydro-4,1-benzoxazepin-1(5H)-yl)methylidene]propanedioic acid diethyl ester

A mixture of Intermediate 15d (2 g, 8.77 mmol) and diethyl ethoxymethylenemalonate (1.77 mL, 8.77 mmol) was heated at 115° C. for 5 h. The reaction mixture was allowed to cool and purified by chromatography eluting with dichloromethane to give the crude title compound (3 g) which was used as such in the next step; ESMS m/z 398.2/400.2 [M+H]⁺.

### f) 10-Bromo-8-oxo-3,4-dihydro-1H,8H-[1,4]oxazepino[6,5,4-ij]quinoline-7-carboxylic acid ethyl ester

A mixture of Intermediate 15e (2 g, 5 mmol) in an excess of polyphosphoric acid was heated to 100° C. for 1 h. Ice was added followed by ultrasonication and dichloromethane extraction. The organic phase was dried then concentrated during this process some of the title compound (0.96 g) precipitated out. Filtration provided a white solid; ESMS m/z 352.2/354.2 [M+H]⁺. Further material (0.3 g) was obtained from the dichlormethane residues by chromatography (silica 0 to 3% methanol in dichloromethane).

### g) 10-Bromo-8-oxo-3,4-dihydro-1H,8H-[1,4]oxazepino[6,5,4-ij]quinoline-7-carboxylic acid

A solution of Intermediate 15f (1.17 g, 3.32 mmol) in 1,4-dioxan (40 mL) was treated with 2M sodium hydroxide (1.83 mL). After stirring at 50° C. for 17 h the temperature was increased to 60° C. for 2.5 h then to 70° C. for 3 h. The mixture was treated with solid carbon dioxide and concentrated to give the title compound as a white solid (1.3 g); ESMS m/z 324.1/326.1 [M+H]⁺.

### h) 10-[3-({[(1,1-Dimethylethyl)oxy]carbonyl}amino)-1-propyn-1-yl]-8-oxo-3,4-dihydro-1H,8H-[1,4]oxazepino[6,5,4-ij]quinoline-7-carboxylic acid

A suspension of Intermediate 15 g (1.07 g, 3.3 mmol), copper (I) iodide (0.063 g, 0.33 mmol) and triethylamine (13 mL) in dry DMF (22 mL) was ultrasonicated whilst argon was bubbled through. After 0.5 h, dichlorobis(triphenylphosphine)palladium (II) (0.070 g, 0.097 mmol) and N-tert-butoxycarbonylpropargylamine (0.82 g, 5.3 mmol) were added and the mixture heated at 100° C. for 1.5 h. The reaction mixture was cooled, concentrated in vacuo and purified by chromatography (silica gel, 0 to 10% [9:1 methanol/0.880 ammonia] in dichloromethane to give, after trituration with diethyl ether, the title compound (0.8 g) as a white solid; ESMS m/z 399.3 [M+H]⁺.

### i) 10-[3-({[(1,1-Dimethylethyl)oxy]carbonyl}amino)propyl]-8-oxo-3,4-dihydro-1H,8H-[1,4]oxazepino[6,5,4-ij]quinoline-7-carboxylic acid

A solution of Intermediate 15 h (0.8 g, 2 mmol) in DMF (20 mL) was treated with 10% palladium on carbon (0.6 g) and hydrogenated at room temperature and atmospheric pressure for 17 h. The reaction mixture was filtered through celite with some charcoal and concentrated to give the title compound (0.5 g) as a white solid; ESMS m/z 403.3 [M+H]⁺.

### j) 10-(3-Aminopropyl)-8-oxo-3,4-dihydro-1H,8H-[1,4]oxazepino[6,5,4-ij]quinoline-7-carboxylic acid

A solution of Intermediate 15i (0.5 g, 1.24 mmol) in dichloromethane (20 mL) was treated with trifluoroacetic acid (10 mL) and the reaction stirred at room temperature for 20 min. The solution was evaporated to dryness and triturated with diethyl ether to give the title compound (0.53 g) as a white solid; ESMS m/z 303.3 [M+H]⁺.

### Intermediate 16: 10-[(2-aminoethyl)thio]-8-oxo-3,4-dihydro-2H,8H-[1,4]oxazepino[2,3,4-ij]quinoline-7-carboxylic acid

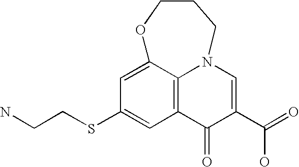

### a) 3-[(3-Hydroxypropyl)amino]-2-[(2,3,5-trifluorophenyl)carbonyl]-2-propenoic acid ethyl ester

Using a similar procedure to that described in Intermediate 14b, 3-amino-1-propanol (0.83 g, 11.0 mmol) and Intermediate 14a (3.00 g, 10.0 mmol) gave the title compound as a yellow oil (2.93 g); ESMS m/z 354.3 [M+Na]⁺.

### b) 10-Fluoro-8-oxo-3,4-dihydro-2H,8H-[1,4]oxazepino[2,3,4-ij]quinoline-7-carboxylic acid ethyl ester

Using a similar procedure to that described in Intermediate 14c, DBU (1.3 mL, 8.78 mmol) and Intermediate 16a (2.91 g, 8.78 mmol) gave the title compound as a white solid (1.79 g); ESMS m/z 292.3 [M+H]⁺.

### c) 10-{[2-({[(1,1-Dimethylethyl)oxy]carbonyl}amino)ethyl]thio}-8-oxo-3,4-dihydro-2H,8H-[1,4]oxazepino[2,3,4-ij]quinoline-7-carboxylic acid ethyl ester

Using a similar procedure to that described in Intermediate 14d, Intermediate 16b (1.76 g, 6.04 mmol) gave the title compound (1.68 g); ESMS m/z 449.4 [M+H]⁺.

### d) 10-[(2-Aminoethyl)thio]-8-oxo-3,4-dihydro-2H,8H-[1,4]oxazepino[2,3,4-ij]quinoline-7-carboxylic acid

Using a similar procedure to that described in Intermediate 14e, Intermediate 16c (1.68 g, 3.75 mmol) gave the title compound as a white solid (1.68 g); ESMS m/z 321.3 [M+H]⁺.

### Intermediate 17: 9-[2-(2-Aminoethoxy)ethylthio]-3,3-dimethyl-7-oxo-1H,7H-[1,3]oxazino[5,4,3-ij]quinolone-6-carboxylic acid trifluoroacetic acid salt

### a) 2-Amino-5-iodo-benzoic acid methyl ester

2-Amino-5-iodo-benzoic acid (10.00 g) was dissolved in methanol (200 mL). To this solution, Amberlist-15 (16.00 g) was added and the resulting mixture was heated under reflux under nitrogen for 48 h. After cooling to room temperature the mixture was filtered and the solid was washed with 5% 20M ammonia/MeOH (2×100 mL). The organic phase was dried and the solvent evaporated in vacuo. The residue was purified by flash chromatography (silica gel, ethyl acetate/hexane from 1/15 to 1/12) to give the title compound (7.00 g); ESMS m/z 277.9 [M+H]⁺.

### b) (2-Amino-5-iodo-phenyl)-methanol

Intermediate 17a (14.00 g) was dissolved in dry THF (50 mL) and 1M diisobutylaluminium hydride in THF (165 mL) was added dropwise to this solution at −30° C. The solution was then stirred at −15° C. for 1 h. To this mixture MeOH (50 mL) was added portionwise maintaining the temperature of the solution below −5° C. The resulting mixture was then at −15° C. for 18 h. The formed solid was filtered off and washed with MeOH. The organic phase was dried and the solvent was evaporated in vacuo. The residue was purified by flash chromatography (silica gel, 1-5% methanol in dichloromethane) to give the title compound (9.70 g); ESMS m/z 249.9 [M+H]⁺.

### c) 2-[(2-Hydroxymethyl-4-iodo-phenylamino)methylene]malonic acid diethyl ester

Intermediate 17b (9.70 g) and 2-ethoxymethylene-malonic acid diethyl ester (8.50 g) were mixed and heated at 110° C. in an open vessel under microwave heating for 20 min. After cooling the resulting solid was triturated with diethyl ether and filtered to give the title compound (9.78 g); ESMS m/z 419.8 [M+H]⁺.

### d) 2-[(2-Acetoxymethyl-4-iodo-phenylamino)methylene]malonic acid diethyl ester

To a solution of Intermediate 17c (9.78 g) and triethylamine (3.6 mL) in dry dichloromethane (100 mL) acetic anhydride (2.43 mL) was added at room temperature and the solution was stirred for 18 h. The solution was washed with water (2×50 mL) and with brine (50 mL). The organic phase was dried and the solvent was removed in vacuo to give the title compound (12.00 g) as a white solid; ESMS m/z 461.8 [M+H]⁺.

### e) 8-Acetoxymethyl-6-iodo-4-oxo-1,4-dihydro-quinoline-3-carboxylic acid ethyl ester

Intermediate 17d (12.00 g) was dissolved in diphenylether (25 mL) and heated at 230° C. for 45 min under microwave heating in an open vessel. The crude was triturated with diisopropyl ether to give a first batch of the title product. A second batch was obtained by flash chromatography of the mother liquors (silica gel, 0-3% methanol in dichloromethane) to obtain the title product (total 4.50 g); ESMS m/z 415.9 [M+H]⁺.

### f) 8-Hydroxymethyl-6-iodo-4-oxo-1,4-dihydro-quinoline-3-carboxylic acid ethyl ester

To a solution of Intermediate 17e (4.50 g) in EtOH (60 mL) a solution of sodium ethylate (0.330 g of sodium in 30 mL of EtOH) was added dropwise at 0° C. The resulting solution was stirred at room temperature for 1.5 h. The solution was concentrated in vacuo and the mixture was cooled at −15° C. for 18 h. The precipitated solid was filtered and washed with diisopropyl ether to give the title compound (2.86 g); ESMS m/z 373.9 [M+H]⁺.

### g) 9-Iodo-3,3-dimethyl-7-oxo-1H,7H-[1,3]oxazino[5,4,3-ij]quinolone-6-carboxylic acid ethyl ester

To a solution of 2,2-dimethoxypropane (12.4 mL) in dry DMF (12 mL) hydrated p-toluenesulfonic acid (0.83 g) and molecular sieves 3A (1.7 g) were added under nitrogen. Intermediate 17f (1.25 g) was added and the mixture was stirred at reflux under nitrogen for 1.5 h. The excess of 2,2-dimethoxypropane was evaporated, dichloromethane (15 mL) was added and the resulting solution was extracted with aqueous 5% NaHCO₃ (2×15 mL) and brine (15 mL). The organic phase was dried and the solvent was evaporated in vacuo. The residue was triturated with diisopropyl ether to give the title compound (1.03 g); ESMS m/z 413.8 [M+H]⁺.

### h) [2-(2-Hydroxyethoxy)ethyl]-arbamic acid tert-butyl ester

To a solution of 2-(2-aminoethoxy)ethanol (5.00 g) in water (35 mL) potassium hydroxide (2.93 g) was added at 0° C. To this mixture maintained at 0° C., a solution of di-tert-butyldicarbonate (11.40 g) in dioxane (17 mL) was added dropwise. The resulting mixture was stirred at 0° C. for 1 h and then 4 h at room temperature. The dioxane was evaporated and the aqueous solution was extracted with dichloromethane (2×25 mL). The organic phase was dried and evaporated in vacuo. The residue was purified by flash chromatography (silica gel, ethyl acetate/petroleum ether 40/60 to 60/40) to give the title product (8.50 g); ESMS m/z 205.3 [M+H]⁺.

### i) Toluene-4-sulfonic acid 2-(2-tert-butoxycarbonylamino-ethoxy)-ethyl ester

Intermediate 17 h (7.39 g) was dissolved in dichloromethane (75 mL) and to the solution triethylamine (5.00 mL) and p-toluenesulfonyl chloride (6.87 g) were added at 0° C. The resulting solution was stirred at room temperature overnight. The suspension was filtered off and the organic phase was evaporated in vacuo. The residue was purified by flash chromatography (silica gel, ethyl acetate/petroleum ether from 20/80 to 40/60) to give the title product (11.25 g); ESMS m/z 360.4 [M+H]⁺.

### j) Thioacetic acid S-[2-(2-tert-butoxycarbonylamino-ethoxy)-ethyl]ester

To a solution of Intermediate 17i (11.69 g) in acetone (250 mL) potassium thioacetate (7.42 g) was added; the resulting suspension was heated at reflux for 2 h. The solid was filtered off and washed with acetone. The solution was evaporated in vacuo and the residue was purified by filtration on a short silica pad (ethyl acetate/petroleum ether 20/80) to obtain the title compound (6.85 g); ESMS m/z 264.4 [M+H]⁺.

### k) 9-[2-(2-tert-Butoxycarbonylaminoethoxy)ethylthio]-3,3-dimethyl-7-oxo-1H,7H-[1,3]oxazino[5,4,3-ij]quinolone-6-carboxylic acid

A suspension of Intermediate 17 g (1.00 g), Intermediate 17j (0.64 g), copper (I) iodide (0.019 g), potassium carbonate (0.69 g) and ethylene glycol (0.28 mL) in absolute ethanol (10 mL) was degassed by nitrogen bubbling for 30 min. The mixture was then heated in a sealed tube at 120° C. for 1 h under microwave irradiation. The solvent was removed in vacuo; the residue was dissolved in dichloromethane (10 mL) and extracted with 20% aqueous citric acid. The organic phase was dried and evaporated in vacuo. The residue was purified by flash chromatography (silica gel, 0-3% methanol in dichloromethane) to give the title compound (0.85 g); ESMS m/z 478.8 [M+H]⁺.

### l) 9-(Aminoethoxy)ethylthio]-3,3-dimethyl-7-oxo-1H,7H-[1,3]oxazino[5,4,3-ij]quinolone-6-carboxylic acid ethyl ester trifluoroacetic acid salt

Intermediate 17k (0.53 g) was dissolved in dry dichloromethane (2 mL) and freshly distilled trifluoroacetic acid (1.0 mL) was added at room temperature. The resulting solution was stirred at room temperature under nitrogen for 2 h. The solvent was removed in vacuo and the residue was triturated with dry THF to give the title compound (0.45 g); ESMS m/z 378.9 [M+H]⁺.

### Intermediate 18: 9-[3-(2-Aminoethoxy)propyl]-3,3-dimethyl-7-oxo-1H,7H-2-[1,3]oxazino[5,4,3-ij]quinolone-6-carboxylic acid trifluoroacetic acid salt

### a) (2-Prop-2-ynyloxyethyl)carbamic acid tert-butyl ester

To a solution of (2-hydroxyethyl)carbamic acid tert-butyl ester (1.56 g) in dichloromethane (25 mL) benzyltrimethylammonium chloride (0.18 g) was added. To this solution aqueous 50% NaOH (30 g) and propargyl bromide (1.05 mL of 80% solution in toluene) were added and the mixture was vigorously stirred at room temperature for 3 h. The phases were diluted with dichloromethane and separated. The organic phase was washed with water (2×15 mL), dried and evaporated in vacuo. The residue was purified by flash chromatography (silica gel, 0-5% methanol in dichloromethane) to give the title compound as a pale-orange oil (1.53 g); ESMS m/z 222.4 [M+Na]⁺.

### b) 9-[3-(2-tert-Butoxycarbonylaminoethoxy)prop-1-ynyl]-3,3-dimethyl-7-oxo-1H,7H-2-[1,3]oxazino[5,4,3-ij]quinolone-6-carboxylic acid ethyl ester

Intermediate 17 g (1.95 g) and copper (I) iodide (0.031 g) were suspended in a mixture of acetonitrile (50 mL) and triethylamine (25 mL); the suspension was degassed by nitrogen bubbling for 30 min. Intermediate 18a (1.56 g) and dichlorobis(triphenylphosphine)palladium (II) (0.115 g) were added under nitrogen and the mixture was heated at 100° C. for 2 h. After this period the solvent was removed in vacuo and the residue, dissolved in dichloromethane (100 mL), was washed with water (2×50 mL). The organic phase was dried and evaporated in vacuo. The residue was purified by flash chromatography (silica gel, 0-2% methanol in dichloromethane) to give the title compound (1.63 g); ESMS m/z 485.5 [M+H]⁺.

### c) 9-[3-(2-tert-Butoxycarbonylaminoethoxy)propyl]-3,3-dimethyl-7-oxo-1H,7H-2-[1,3]oxazino[5,4,3-ij]quinolone-6-carboxylic acid ethyl ester

Intermediate 18b (1.63 g) was dissolved in dichloromethane (40 mL) and 10% palladium on carbon (0.350 g) was added. The mixture was hydrogenated at room temperature at 25 psi for 18 h. The catalyst was filtered and the solvent evaporated in vacuo to give the title compound (1.71 g); ESMS m/z 489.5 [M+H]⁺.

### d) 9-[3-(2-tert-Butoxycarbonylaminoethoxy)propyl]-3,3-dimethyl-7-oxo-1H,7H-2-[1,3]oxazino[5,4,3-ij]quinolone-6-carboxylic acid

To a solution of Intermediate 18c (1.71 g) in dioxane (15 mL) 2N NaOH (3.3 mL) was added. The resulting mixture was stirred at room temperature for 3 h. The solvent was evaporated in vacuo and the crude was partitioned between water and dichloromethane. Formic acid (0.5 mL) was added to adjust pH of the aqueous phase to 4. The organic phase was dried and evaporated in vacuo, to give the title compound (1.54 g); ESMS m/z 461.5 [M+H]⁺.

### e) 9-[3-(2-Aminoethoxy)propyl]-3,3-dimethyl-7-oxo-1H,7H-2-[1,3]oxazino[5,4,3-ij]quinolone-6-carboxylic acid trifluoroacetic acid salt

A solution of Intermediate 18d (0.28 g) in dry dichloromethane (1.5 mL) was treated with freshly distilled trifluoroacetic acid (0.6 mL) and stirred at room temperature under nitrogen for 2 h. The solvent was evaporated in vacuo; the residue was re-evaporated from dichloromethane (2×) and triturated with dry THF to give the title compound as a solid (0.20 g); ESMS m/z 361.4 [M+H]⁺.

### Intermediate 19: 9-[3-(2-Aminoethoxy)propyl]-7-oxo-1H,7H-2-[1,3]oxazino[5,4,3-ij]quinolone-6-carboxylic acid trifluoroacetic acid

### a) 9-Iodo-7-oxo-1H,7H-2-[1,3]oxazino[5,4,3-ij]quinolone-6-carboxylic acid ethyl ester

To a solution of Intermediate 17f (5.71 g) in dry DMF (40 mL), anhydrous potassium carbonate (6.34 g) and chloroiodomethane (11.1 mL) were added and the resulting suspension was stirred at 100° C. under nitrogen for 18 h. The solvent was evaporated in vacuo and the residue was purified by flash chromatography (silica gel, 0-2% methanol in dichloromethane) to give the title compound (2.24 g); ESMS m/z 385.8 [M+H]⁺.

### b) 9-[3-(2-tert-Butoxycarbonylaminoethoxy)prop-1-ynyl]-6-oxo-1H,7H-2-[1,3]oxazino[5,4,3-ij]quinolone-6-carboxylic acid ethyl ester

Intermediate 19a (2.2 g) and copper (I) iodide (0.032 g) were suspended in a mixture of CH₃CN (50 mL) and triethylamine (25 mL); the suspension was degassed by nitrogen for 30 min. Intermediate 18a (1.71 g) and dichlorobis(triphenylphosphine)palladium (II) (0.120 g) were added under nitrogen and the mixture was heated at reflux for 2 h. After this period solvent was removed and dichloromethane (50 mL) was added and washed with aqueous 5% citric acid (2×20 mL). The organic phase was dried and evaporated in vacuo. The residue was purified by flash chromatography (silica gel, 0-2% methanol in dichloromethane) to give a brown solid that was triturated with diisopropyl ether to give the title compound (0.85 g); ESMS m/z 456.9 [M+H]⁺.

### c) 9-[3-(2-tert-Butoxycarbonylaminoethoxy)propyl]-7-oxo-1H,7H-2-[1,3]oxazino[5,4,3-ij]quinolone-6-carboxylic acid ethyl ester

Intermediate 19b (0.85 g) was dissolved in EtOH (30 mL) and wet (50% dispersion in water) 10% palladium on carbon (0.16 g) was added. The mixture was hydrogenated at room temperature at 30 psi for 2.5 h. The catalyst was filtered off and the solvent evaporated in vacuo to give the title compound (0.65 g); ESMS m/z 460.9 [M+H]⁺.

### d) 9-[3-(2-tert-Butoxycarbonylaminoethoxy)propyl]-7-oxo-1H,7H-2-[1,3]oxazino[5,4,3-ij]quinolone-6-carboxylic acid

To a solution of Intermediate 19c (0.65 g) in THF (8 mL) 2N aqueous NaOH (1.5 mL) was added. The resulting mixture was heated at reflux for 3 h. The mixture was acidified with 5% aqueous citric acid and extracted with dichloromethane (3×30 mL). The organic phase was dried and evaporated in vacuo, to give the title compound (0.58 g); ESMS m/z 432.9 [M+H]⁺.

### e) 9-[3-(2-Aminoethoxy)propyl]-7-oxo-1H,7H-2-[1,3]oxazino[5,4,3-ij]quinolone-6-carboxylic acid trifluoroacetic acid salt

A solution of Intermediate 19d (0.58 g) in dichloromethane (15 mL) was treated with trifluoroacetic acid (1 mL) and stirred at room temperature for 4 h. The solvent was evaporated in vacuo; the residue was re-evaporated from dichloromethane (2×) and triturated with diisopropylether to give the title compound as a solid (0.55 g); ESMS m/z 333.0 [M+H]⁺.

### Intermediate 20: 2′-O-Acetyl-4″-O-(imidazol-1-yl-carbonyl)-erythromycin A-9(E)-O-(2-diethylaminoethyl)-oxime

### a) 2′-O-Acetyl-erythromycin A-9(E)-O(2-diethylaminoethyl)-oxime

To a solution of erythromycin A-9(E)-O(2-diethylaminoethyl)-oxime (4.05 g) in dichloromethane (50 mL) sodium hydrogen carbonate (0.60 g) was added followed by acetic anhydride (0.68 mL). After stirring overnight at room temperature the mixture was diluted with dichloromethane and washed with 5% aqueous sodium carbonate. The organic layer was separated, dried and evaporated to yield the title product (4.10 g) as a solid; ESMS m/z 890.3 [M+H]⁺.

### b) 2′-O-Acetyl-4″-O-(imidazol-1-yl-carbonyl)-erythromycin A-9(E)-O(2-diethylaminoethyl)-oxime

To a solution of Intermediate 20a (3.70 g) in dry THF (125 mL) 4-dimethylaminopyridine (2.54 g) and carbonyl diimidazole (3.37 g) were added. The resulting mixture was stirred under nitrogen at room temperature for 6 h. The solvent was removed in vacuo and the residue was dissolved in ethyl acetate (150 mL) and washed with 5% aqueous potassium dihydrogen phosphate (2×100 mL) and brine (100 mL). The organic phase was dried and evaporated in vacuo to give the title compound (4.10 g) as white foam; ESMS m/z 984.3 [M+H]⁺.

### Intermediate 21: 2′-O-Acetyl-4″-O-(imidazol-1-yl-carbonyl)-erythromycin A-9(E)-O-methoxymethyloxime

### a) 2′-O-Acetyl-erythromycin A-9(E)-O-methoxymethyloxime

To a solution of erythromycin A-9(E)-O-methoxymethyloxime (4.05 g) in dichloromethane (50 mL) sodium hydrogen carbonate (0.60 g) was added followed by acetic anhydride (0.68 mL). After stirring overnight at room temperature the mixture was diluted with dichloromethane and washed with 5% aqueous sodium hydrogen carbonate. The organic layer was separated, dried and evaporated to yield the title product (4.10 g) as a solid; ESMS m/z 835.8 [M+H]⁺.

### b) 2′-O-Acetyl-4″-O-(imidazol-1-yl-carbonyl)-erythromycin A-9(E)-O-methoxymethyloxime

To a solution of Intermediate 21a (1.10 g) in dry THF (40 mL) 4-dimethylaminopyridine (0.80 g) and carbonyl diimidazole (1.10 g) were added. The resulting mixture was stirred under nitrogen at room temperature for 6 h. To the solution ethyl acetate and 5% aqueous potassium hydrogen phosphate were added (100 mL each) and the phases were separated. The organic phase was dried and evaporated in vacuo to give the title compound (1.10 g) as white foam; ESMS m/z 929.9 [M+H]⁺.

### Intermediate 22: 2′-O-Acetyl-4″-O-(imidazol-1-yl-carbonyl)-erythromycin A-9(E)-O-methyloxime

### a) 2′-O-Acetyl-erythromycin A-9(E)-O-methyloxime

To a solution of erythromycin A-9(E)-O-methyloxime (7.65 g) in dichloromethane (100 mL) sodium hydrogen carbonate (1.26 g) was added followed by acetic anhydride (1.42 mL). After stirring overnight at room temperature the mixture was diluted with dichloromethane and washed with 5% aqueous sodium hydrogen carbonate. The organic layer was separated, dried and evaporated to yield the title product (7.50 g) as a solid; ESMS m/z 805.6 [M+H]⁺.

### b) 2′-O-Acetyl-4″-O-(imidazol-1-yl-carbonyl)-erythromycin A-9(E)-O-methyloxime

To a solution of Intermediate 22a (0.50 g) in dry THF (20 mL) 4-dimethylaminopyridine (0.38 g) and carbonyl diimidazole (0.50 g) were added; the resulting mixture was stirred under nitrogen at room temperature for 6 h. To the solution ethyl acetate and 5% aqueous potassium dihydrogen phosphate were added (50 mL each) and the phases were separated. The organic phase was dried and evaporated in vacuo to give the title compound (0.50 g) as white foam; ESMS m/z 899.8 [M+H]⁺.

### Intermediate 23: 2′-O-Acetyl-4″-O-(imidazol-1-yl-carbonyl)-erythromycin A-9(E)-O-(1-methoxy-1-methylethyl)-oxime

### a) 2′-O-Acetyl-erythromycin A-9(E)-O-(1-methoxy-1-methylethyl)-oxime

To a solution of erythromycin A-9(E)-O(1-methoxy-1-methylethyl)-oxime (0.86 g) in dichloromethane (15 mL) sodium hydrogen carbonate (0.13 g) was added followed by acetic anhydride (0.15 mL). After stirring overnight at room temperature the mixture was diluted with dichloromethane and washed with 5% aqueous sodium hydrogen carbonate The organic layer was separated, dried and evaporated to yield the title product (0.90 g) as a solid; ESMS m/z 863.4 [M+H]⁺.

### b) 2′-O-Acetyl-4″-O-(imidazol-1-yl-carbonyl)-erythromycin A-9(E)-O-(1-methoxy-1-methylethyl)-oxime

To a solution of Intermediate 23a (0.90 g) in dry THF (25 mL) 4-dimethylaminopyridine (0.70 g) and carbonyl diimidazole (0.90 g) were added. The resulting mixture was stirred under nitrogen at room temperature for 6 h. To the solution ethyl acetate and 5% aqueous potassium dihydrogen phosphate were added (100 mL each) and the phases were separated. The organic phase was dried and evaporated in vacuo to give the title compound (0.95 g) as white foam; ESMS m/z 957.4 [M+H]⁺.

### Intermediate 24: 2′-O-Acetyl-4″-O-(imidazol-1-VI-carbonyl)-erythromycin A-9(E)-O-cyanomethyloxime

### a) Erythromycin A-9(E)-O-cyanomethyloxime

To a solution of erythromycin A-9-oxime (3.00 g) in dry THF (120 mL) a solution of tetrabutylammonium hydroxide 1M in methanol (4.4 mL) was added at room temperature. After 5 min chloroacetonitrile (0.38 mL) was added dropwise and the resulting mixture was heated at 50° C. for 1 h. The solvent was removed in vacuo and the residue was dissolved in diethyl ether and washed with brine (3×75 mL). The organic phase was dried and evaporated in vacuo. The residue was purified by flash chromatography (silica gel, 0-3% of 9/1 methanol/20M ammonia in dichloromethane) to give the title compound (2.27 g) as a solid; ESMS m/z 788.4 [M+H]⁺.

### b) 2′-O-Acetyl-erythromycin A-9(E)-O-cyanomethyloxime

To a solution of Intermediate 24a (2.24 g) in dichloromethane (50 mL) sodium hydrogen carbonate (0.36 g) was added followed by acetic anhydride (0.40 mL). After stirring overnight at room temperature the mixture was diluted with dichloromethane and washed with 5% aqueous sodium hydrogen carbonate. The organic layer was separated, dried and evaporated to yield the title product (2.35 g) as a solid; ESMS m/z 830.4 [M+H]⁺.

### c) 2′-O-Acetyl-4″-O-(imidazol-1-yl-carbonyl)-erythromycin A-9(E)-O-cyanomethyloxime

To a solution of Intermediate 24b (2.35 g) in dry THF (90 mL) 4-dimethylaminopyridine (1.73 g) and carbonyl diimidazole (2.30 g) were added; the resulting mixture was stirred under nitrogen at room temperature for 6 h. The solvent was removed in vacuo and the residue was dissolved in ethyl acetate (150 mL) and washed with 5% aqueous potassium dihydrogen phosphate (2×100 mL) and brine (100 mL). The organic phase was dried and evaporated in vacuo to give the title compound (2.65 g) as white foam; ESMS m/z 924.4 [M+H]⁺.

### Intermediate 25: 2′-O-Acetyl-4″-O-(imidazol-1-yl-carbonyl)-erythromycin A-9(E)-O-benzyloxime

### a) 2′-O-Acetyl-erythromycin A-9(E)-O-benzyloxime

To a solution of erythromycin A-9(E)-O-benzyloxime (0.83 g) in dichloromethane (23 mL) sodium hydrogen carbonate (0.125 g) was added followed by acetic anhydride (0.140 mL). After stirring overnight at room temperature the mixture was diluted with dichloromethane and washed with 5% aqueous sodium hydrogen carbonate. The organic layer was separated, dried and evaporated to yield the title product (0.85 g) as a solid; ESMS m/z 882.2 [M+H]⁺.

### b) 2′-O-Acetyl-4″-O-(imidazol-1-yl-carbonyl)-erythromycin A-9(E)-O-benzyloxime

To a solution of Intermediate 25a (0.86 g) in dry THF (30 mL) 4-dimethylaminopyridine (0.59 g) and carbonyl diimidazole (0.79 g) were added; the resulting mixture was stirred under nitrogen at room temperature for 6 h. The solvent was removed in vacuo and the residue was dissolved in ethyl acetate (150 mL) and washed with 5% aqueous potassium dihydrogen phosphate (2×100 mL) and brine (100 mL). The organic phase was dried and evaporated in vacuo to give the title compound (0.86 g) as white foam; ESMS m/z 976.0 [M+H]⁺.

### Intermediate 26: 2′-O-Acetyl-4″-O-(imidazol-1-yl-carbonyl)-erythromycin A-9-dihydro-9,11-ethylidene acetal

### a) 2′-O-Acetyl-erythromycin A-9-dihydro-9,11-ethylidene acetal

To a solution of erythromycin A-9-dihydro-9,11-ethylidene acetal (0.95 g) in dichloromethane (30 mL) sodium hydrogen carbonate (0.16 g) was added followed by acetic anhydride (0.18 mL). After stirring overnight at room temperature the mixture was diluted with dichloromethane and washed with 5% aqueous sodium hydrogen carbonate. The organic layer was separated, dried and evaporated to yield the title product (1.00 g) as a solid; ESMS m/z 804.4 [M+H]⁺.

### b) 2′-O-Acetyl-4″-O-(imidazol-1-yl-carbonyl)-erythromycin A-9-dihydro-9,11-ethylidene acetal

To a solution of Intermediate 26a (1.0 g) in dry THF (30 mL) 4-dimethylaminopyridine (0.80 g) and carbonyl diimidazole (1.0 g) were added; the resulting mixture was stirred under nitrogen at room temperature for 6 h. To the solution ethyl acetate and 5% aqueous potassium dihydrogen phosphate were added (100 mL each) and the phases were separated. The organic phase was dried and evaporated in vacuo to give the title compound (1.10 g) as white foam; ESMS m/z 898.6 [M+H]⁺.

### Intermediate 27: 4″-O-(1-Imidazolyl-carbonyl)-6-O-methyl-erythromycin A

6-O-Methyl-erythromycin A (30 g, 40.1 mmol) in THF (100 mL) was treated portionwise with carbonyldiimidazole (16 g, 97 mmol) with ice bath cooling. After 1 h the cooling bath was removed. After a further 48 h, THF (100 mL) and water (200 mL) were added causing slow precipitation of the title compound, which was collected by filtration and dried to give the title compound (24.7 g). Extraction of the mother liquors with diethyl ether gave a further 8.5 g of material which was precipitated from THF solution with water to give a further portion of the title compound as a white solid (3.92 g, total 28.64 g); ESMS m/z 842 [M+H]⁺.

Michael Reaction—General Procedure

Typically a solution of Intermediate 1-5 (0.3 mmol) and Intermediate 6-11 (0.15 mmol) in dimethyl sulfoxide (1.5 mL), triethylamine (0.15 mL) and water (1 drop) was heated at 80° C. for 8-24 h. The mixture was cooled and the resultant solution subjected to mass directed automatic preparative HPLC purification (eluant acetonitrile/water/formic acid) to yield the Michael adduct as the formate salt. The free base was prepared by chromatography over silica gel eluting with 0-10% (9:1 MeOH/20M ammonia) in dichlormethane.

| | Inter- | | | |
|---|---|---|---|---|
| | mediates | Compound Name | Structure | m/z |
| 1 | 1 + 8 | 4″-O-{[(2-{[3-(6-carboxy-3- (R,S)-methyl-7-oxo-1H,7H- [1,3]oxazino[5,4,3- ij]quinolin-9- yl)propyl]amino}propionyl]- 6-O-methyl erythromycin A | 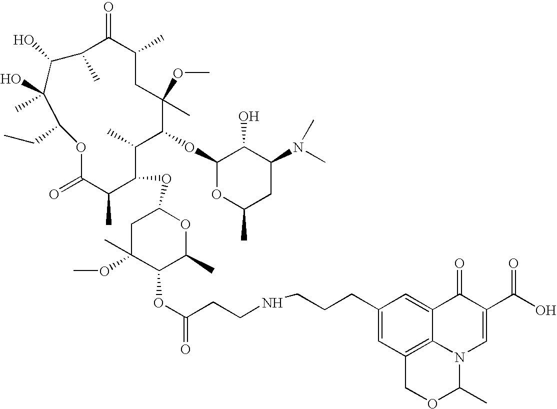 | 1104.6 |
| 2 | 1 + 10 | 4″-O-{[(2-{[3-(6-carboxy-3- (R,S)-methyl-7-oxo-1H,7H- [1,3]oxazino[5,4,3- ij]quinolin-9- yl)propyl]amino}propionyl]- azithromycin | 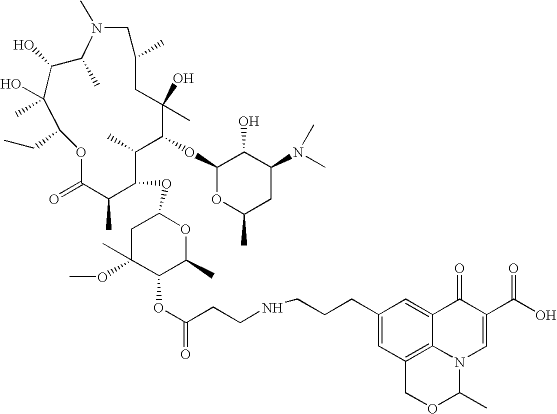 | 1105.7 |
| 3 | 1 + 9 | 4″-O-{[(2-{[3-(6-carboxy-3- (R,S)-methyl-7-oxo-1H,7H- [1,3]oxazino[5,4,3- ij]quinolin-9- yl)propyl]amino}propionyl]- azithromycin-11,12- carbonate | 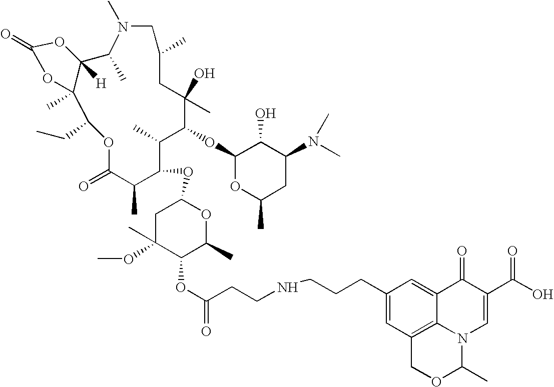 | 1131.8 |
| 4 | 3 + 10 | 4″-O-[(2-{[3-(6-carboxy-3,3- dimethyl-7-oxo-1H,7H- [1,3]oxazino[5,4,3- ij]quinolin-9- yl)propyl]amino}propionyll]- azithromycin | 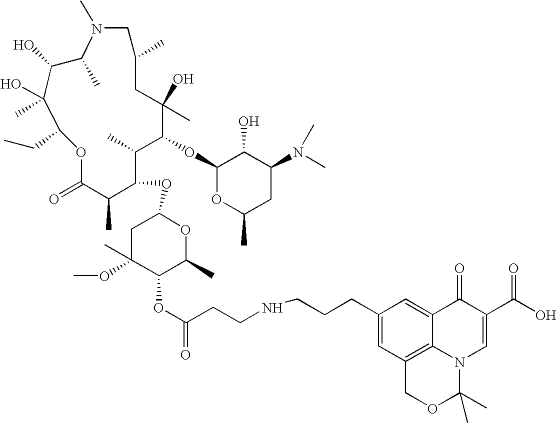 | 1119.8 |
| 5 | 3 + 8 | 4″-O-[(2-{[3-(6-carboxy-3,3- dimethyl-7-oxo-1H,7H- [1,3]oxazino[5,4,3- ij]quinolin-9- yl)propyl]amino}propionyll]- 6-O-methyl erythromycin A | 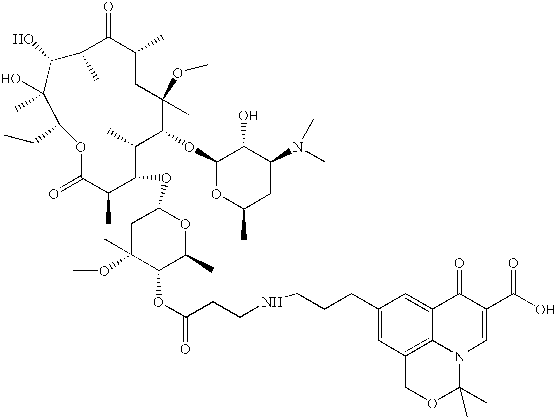 | 1118.7 |
| 6 | 3 + 9 | 4″-O-[(2-{[3-(6-carboxy-3,3- dimethyl-7-oxo-1H,7H- [1,3]oxazino[5,4,3- ij]quinolin-9- yl)propyl]amino}propionyll]- azithromycin-11,12- carbonate | 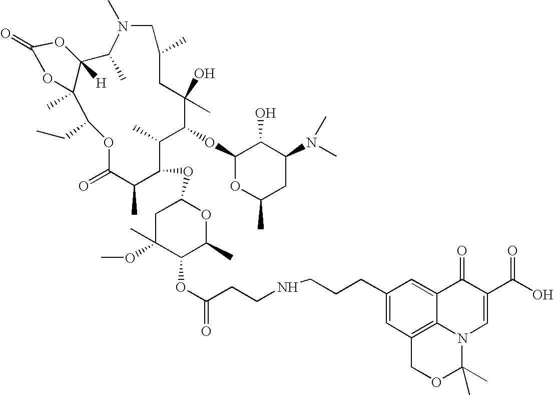 | 1145.8 |
| 7 | 4 + 8 | 4″-O-[(2-{[3-(6-carboxy-7- oxo-1H,7H- [1,3]oxazino[5,4,3- ij]quinolin-9- yl)propyl]amino}propionyll]- 6-O-methyl erythromycin A | 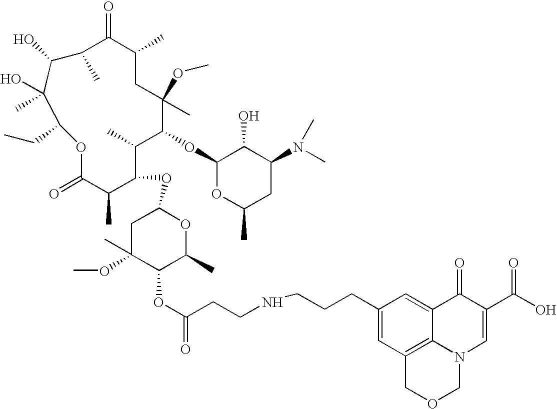 | 1090.7 |
| 8 | 4 + 10 | 4″-O-[(2-{[3-(6-carboxy-7- oxo-1H,7H- [1,3]oxazino[5,4,3- ij]quinolin-9- yl)propyl]amino}propionyll]- azithromycin | 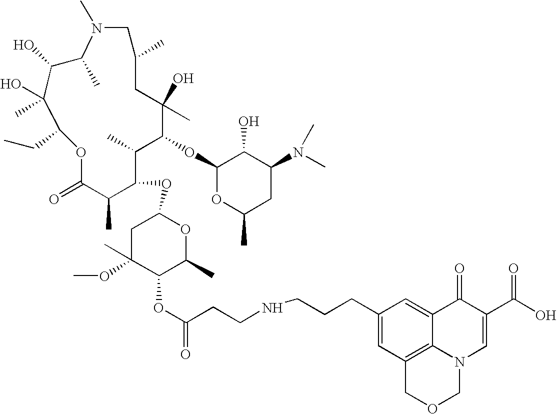 | 1091 .7 |
| 9 | 4 + 9 | 4″-O-[(2-{[3-(6-carboxy-7- oxo-1H,7H- [1,3]oxazino[5,4,3- ij]quinolin-9- yl)propyl]amino}propionyll]- azithromycin-11,12- carbonate | 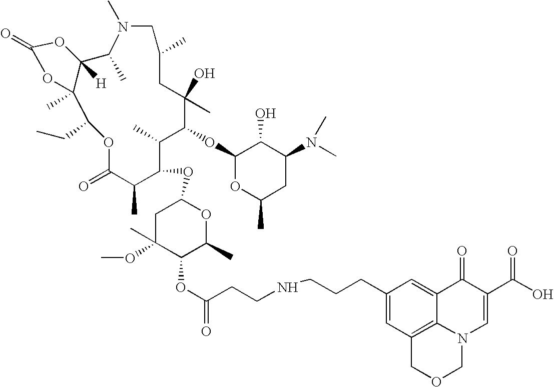 | 1117.8 |
| 10 | 3 + 6 | 4″-O-[(2-{[3-(6-carboxy-3,3- dimethyl-7-oxo-1H,7H- [1,3]oxazino[5,4,3- ij]quinolin-9- yl)propyl]amino}propionyll]- erythromycin A (9E)-oxime | 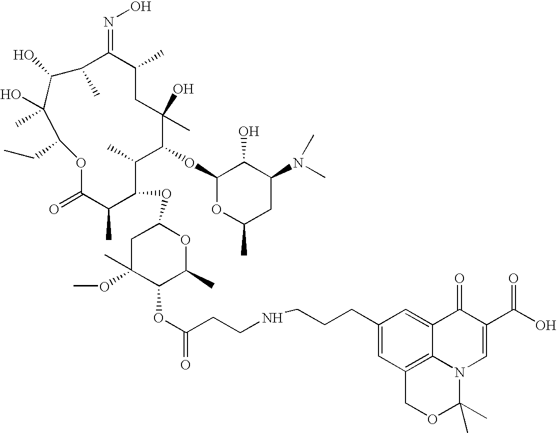 | 1120.0 |
| 11 | 3 + 11 | 4″-O-[(2-{[3-(6-carboxy-3,3- dimethyl-7-oxo-1H,7H- [1,3]oxazino[5,4,3- ij]quinolin-9- yl)propyl]amino}propionyll]- erythromycin A (9E)- methoxymethyl oxime | 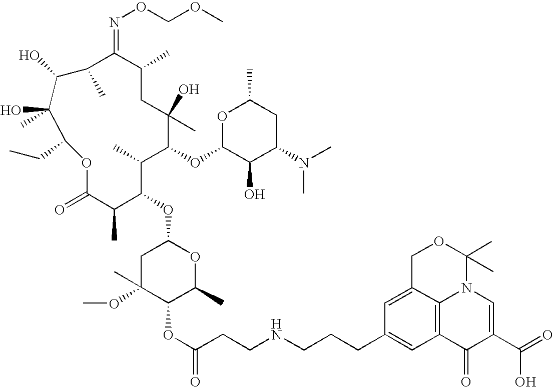 | 1164.1 |
| 12 | 2 + 8 | 4″-O-[(2-{[3-(7-carboxy-8- oxo-3,4-dihydro-2H,8H- [1,4]oxazepino[2,3,4- ij]quinolin-10- yl)propyl]amino}propionyl]- 6-O-methyl erythromycin A | 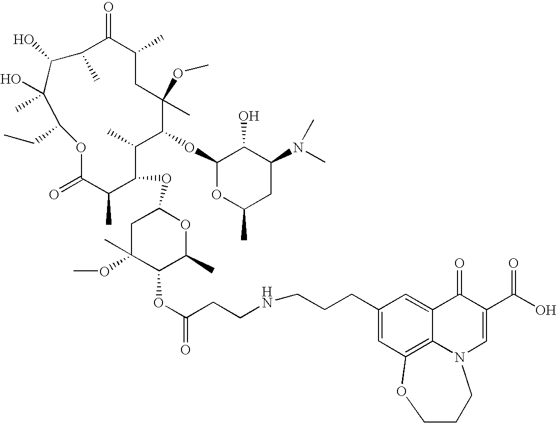 | 1104.9 |
| 13 | 2 + 7 | 4″-O-[(2-{[3-(7-carboxy-8- oxo-3,4-dihydro-2H,8H- [1,4]oxazepino[2,3,4- ij]quinolin-10- yl)propyl]amino}propionyl]- erythromycin (9E)- methoxime | 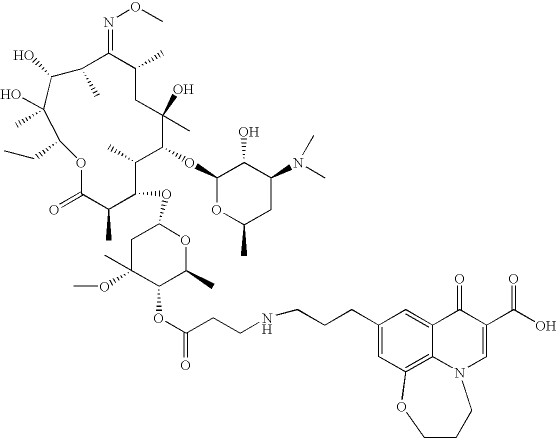 | 1120.0 |
| 14 | 2 + 8 | 4″-O-[(2-{[3-(7-carboxy-8- oxo-3,4-dihydro-2H,8H- [1,4]oxazepino[2,3,4- ij]quinolin-10- yl)propyl]amino}propionyl]- azithromycin-11,12- carbonate | 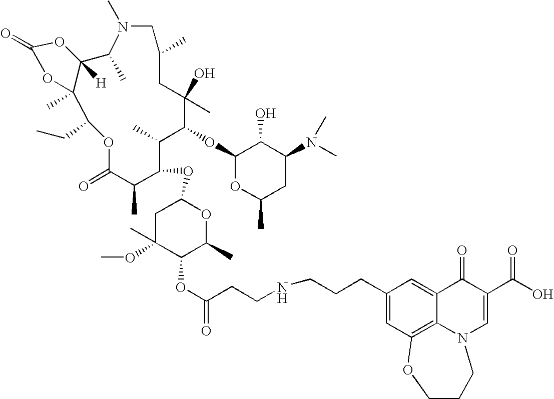 | 1132.0 |
| 15 | 2 + 10 | 4″-O-[(2-{[3-(7-carboxy-8- oxo-3,4-dihydro-2H,8H- [1,4]oxazepino[2,3,4- ij]quinolin-10- yl)propyl]amino}propionyl]- azithromycin | 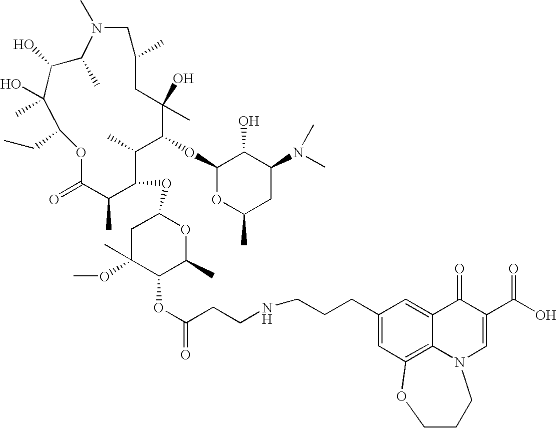 | 1105.9 |
| 16 | 2 + 6 | 4″-O-[(2-{[3-(7-carboxy-8- oxo-3,4-dihydro-2H,8H- [1,4]oxazepino[2,3,4- ij]quinolin-10- yl)propyl]amino}propionyl]- erythromycin (9E)-oxime | 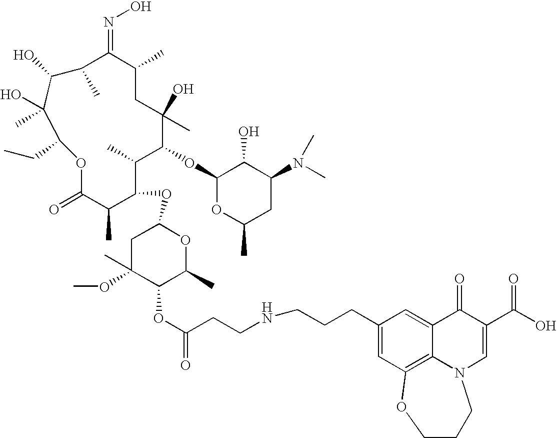 | 1105.9 |
| 17 | 2 + 11 | 4″-O-[(2-{[3-(7-carboxy-8- oxo-3,4-dihydro-2H,8H- [1,4]oxazepino[2,3,4- ij]quinolin-10- yl)propyl]amino}propionyl]- erythromycin (9E)- methoxymethyl xime | 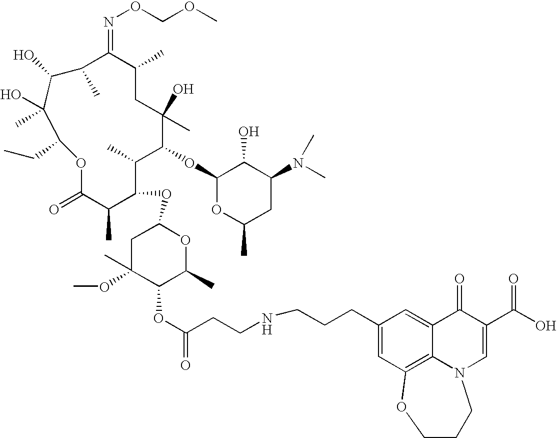 | 1150.0 |
| 18 | 5 + 11 | 4″-O-[(2-{[3-(6-carboxy-3,3- dimethyl-7-oxo-2,3-dihydro- 1H,7H-[1,4]oxazino[2,3,4- ij]quinolin-9- yl)propyl]amino}propionyll]- erythromycin (9E)- methoxymethyl oxime | 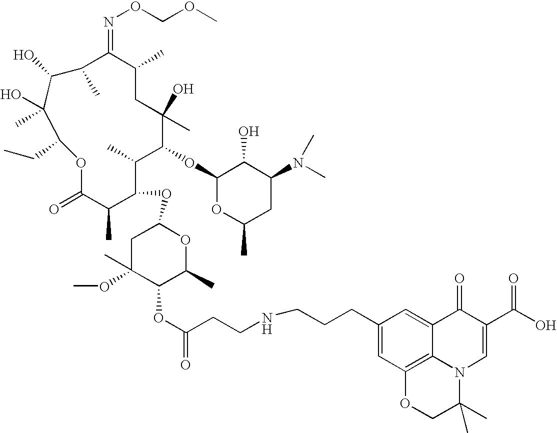 | 1164.0 |
| 19 | 5 + 6 | 4″-O-[(2-{[3-(6-carboxy-3,3- dimethyl-7-oxo-2,3-dihydro- 1H,7H-[1,4]oxazino[2,3,4- ij]quinolin-9- yl)propyl]amino}propionyll]- erythromycin (9E)- 6-O-methyl erythromycin A | 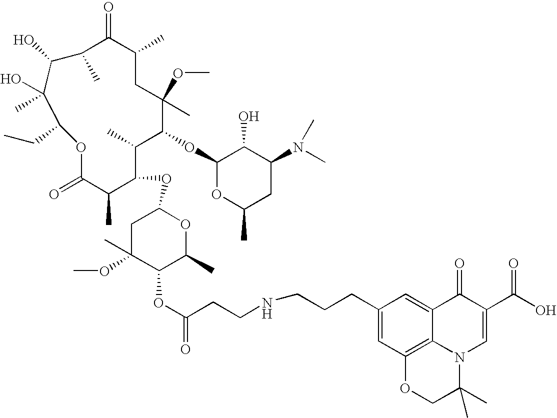 | 1119.0 |
| 20 | 5 + 7 | 4″-O-[(2-{[3-(6-carboxy-3,3- dimethyl-7-oxo-2,3-dihydro- 1H,7H-[1,4]oxazino[2,3,4- ij]quinolin-9- yl)propyl]amino}propionyll]- erythromycin (9E)- erythromycin A (9E)-methoxime | 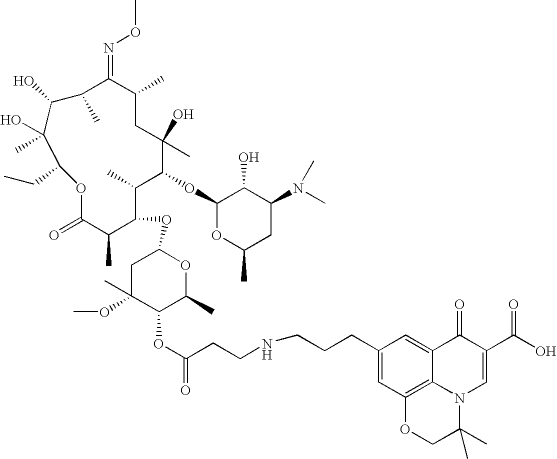 | 1134.0 |
| 21 | 5 + 9 | 4″-O-[(2-{[3-(6-carboxy-3,3- dimethyl-7-oxo-2,3-dihydro- 1H,7H-[1,4]oxazino[2,3,4- ij]quinolin-9- yl)propyl]amino}propionyll]- azithromycin | 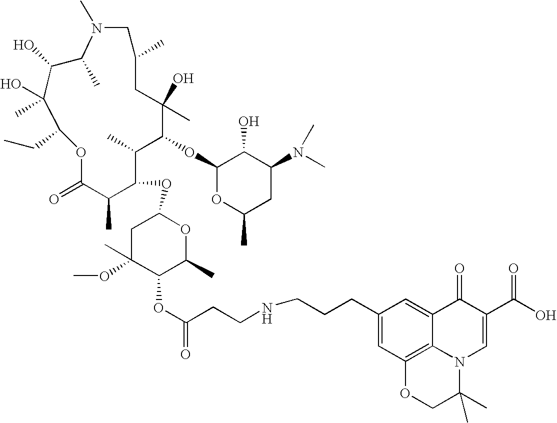 | 1120.0 |

### Example 22

### 4″-O-{2-[3-(6-Carboxy-3,3-dimethyl-7-oxo-1H,7H-2-[1,3]oxazino[5,4,3-ij]quinolin-9-yl)thio)ethoxy]ethylcarbamoyl}-erythromycin A 9(E)-methyloxime trifluoroacetic acid salt

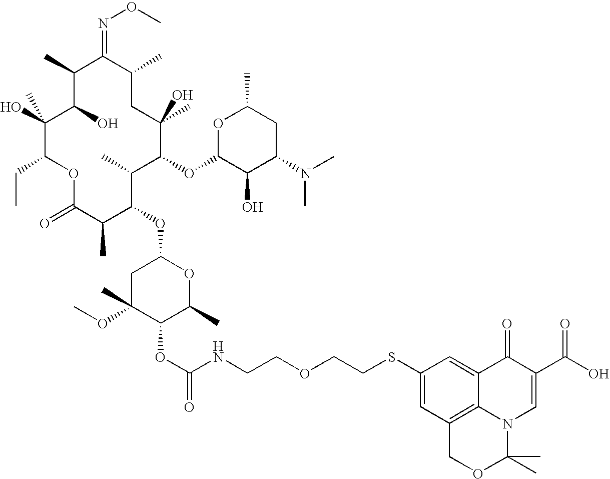

### a) 2′-O-Acetyl-4″-O-{2-[3-(6-Carboxy-3,3-dimethyl-7-oxo-1H,7H-2-[1,3]oxazino[5,4,3-ij]quinolin-9-yl)thio)ethoxy]ethylcarbamoyl}-erythromycin A 9(E)-methyloxime

To a solution of Intermediate 22 (0.225 g) in dry DMF (5 mL) a solution of Intermediate 17 (0.123 g) and DBU (0.150 mL) in dry DMF (5 mL) was added dropwise at room temperature. The resulting mixture was stirred at room temperature for 72 h. Solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 5-10% of 9/1 methanol/0.880 ammonia in dichloromethane) to give the title compound (0.076 g) as white foam; ESMS m/z 1209.3 [M+H]⁺.

### b) 4″-O-{2-[3-(6-Carboxy-3,3-dimethyl-7-oxo-1H,7H-2-[1,3]oxazino[5,4,3-ij]quinolin-9-yl)thio)ethoxy]ethylcarbamoyl}-erythromycin A 9(E)-methyloxime trifluoroacetic acid salt

A solution of Example 22a (0.076 g) in MeOH (5 mL) was stirred at 50° C. overnight. The solvent was removed in vacuo. The residue was purified by preparative reverse phase HPLC to obtain the title compound (0.040 g); ESMS m/z 1167.5 [M+H]⁺.

### Example 23

### 4″-O-[(2-{[3-(6-carboxy-3,3-dimethyl-7-oxo-1H,7H-[1,3]oxazino[5,4,3-ij]quinolin-9-yl)propyl]methylamino}propionyl]-6-O-methyl erythromycin A

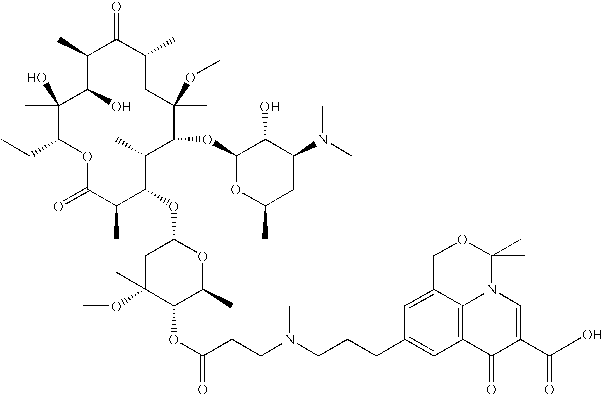

A mixture of Example 5 (0.112 g, 0.1 mmol) in chloroform (1.5 mL), formaldehyde (37% in water, 0.015 mL) and formic acid (0.014 mL) was heated at 60° C. for 2 h. The mixture was concentrated and the residue chromatographed on silica gel eluting with 0-15% (9:1 methanol/0.880 ammonia) in dichloromethane to give the title compound (0.077 g); ESMS m/z 1131.6 [M−H]⁻.

### Example 24

### 4″-O-[(2-{[3-(6-carboxy-3,3-dimethyl-7-oxo-1H,7H-[1,3]oxazino[5,4,3-ij]quinolin-9-yl)propyl]methylamino}ethyl]-6-O-methyl erythromycin A

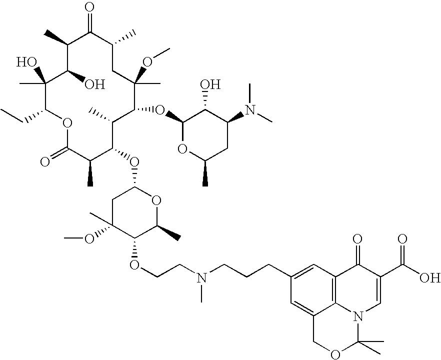

A solution of Intermediate 13 (474 mg, 0.6 mmol) in methanol (10 mL) was added to a solution of the Intermediate 3 (344 mg, 0.8 mmol) and sodium acetate (0.148 g, 1.8 mmol). Acetic acid (0.09 mL) and sodium cyanoborohydride (0.075 g, 1.2 mmol) were added to the resulting solution and the mixture was stirred for 16 h. The reaction was then filtered and concentrated in vacuo to give a residue which was chromatographed on silica gel eluting with 0-15% (9:1 methanol/0.880 ammonia) in dichloromethane to give a mixture of the title compound plus 4″-O-[(2-{[3-(6-carboxy-3,3-dimethyl-7-oxo-1H,7H-[1,3]oxazino[5,4,3-ij]quinolin-9-yl)propyl]amino}ethyl]-6-O-methyl erythromycin A. This mixture (0.1 g, 0.9 mmol) in chloroform (3 mL) was treated with formaldehyde (37% in water, 0.010 mL) and formic acid (0.010 mL) was heated at 60° C. for 4 h. The mixture was concentrated and the residue purified by mass directed automatic preparative HPLC followed by freeze-drying from acetonitrile, water and 0.880 ammonia to give the title compound (0.075 g); ESMS m/z 1104.9 [M+H]⁺

### Example 25

### 4″-O-{3-[2-{[6-carboxy-7-oxo-2,3-dihydro-7H-[1,4]oxazino[2,3,4-ij]quinolin-9-yl]thio}ethylamino]propionyl}-erythromycin A 9(E)-oxime

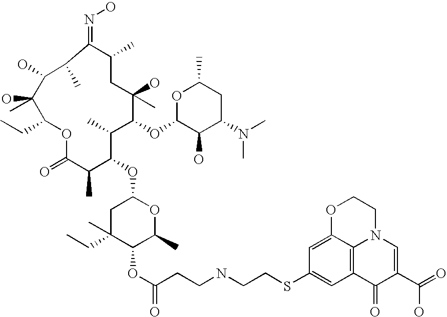

A mixture of Intermediate 6 (0.15 g, 0.187 mmol), Intermediate 14 (0.103 g, 0.299 mmol) and triethylamine (0.4 mL) in DMSO (3 mL) were stirred together at 80° C. for 16 hours. The reaction was concentrated and the residue purified by chromatography (silica gel, 0 to 15% [9:1 methanol/0.880 ammonia] in dichloromethane) to give the title compound (0.062 g) as a white solid; ESMS m/z 1109.9 [M+H]⁺.

### Example 26

### 4″-O-[3-{3-(7-Carboxy-3,4-dihydro-8-oxo-1H,8H-[1,4]oxazepino[6,5,4,-ij]quinolin-10-yl)propylamino}propionyl]-6-O-methyl erythromycin A

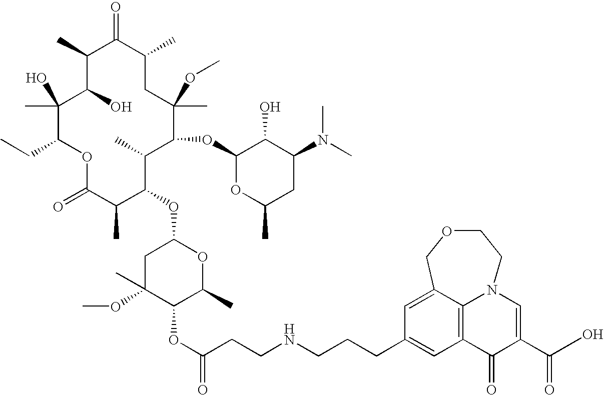

Intermediate 15 (0.105 g, 0.25 mmol), Intermediate 8 (0.160 g, 0.2 mmol) and triethylamine (0.1 mL, 0.7 mmol) in DMSO (0.8 mL) were heated at 80° C. After 16 h the reaction mixture was concentrated and chromatographed over silica gel eluting with 0-15% (9:1 MeOH/0.880M ammonia) in dichloromethane to yield the title compound (0.09 g) as a white solid; ESMS m/z 1104.8 [M+H]⁺.

### Example 27

### 4″-O-[3-{3-(7-Carboxy-3,4-dihydro-8-oxo-1H,8H-[1,4]oxazepino[6,5,4,-ij]quinolin-10-yl)propylamino}propionyl]erythromycin A (9E)-methoxymethyl oxime

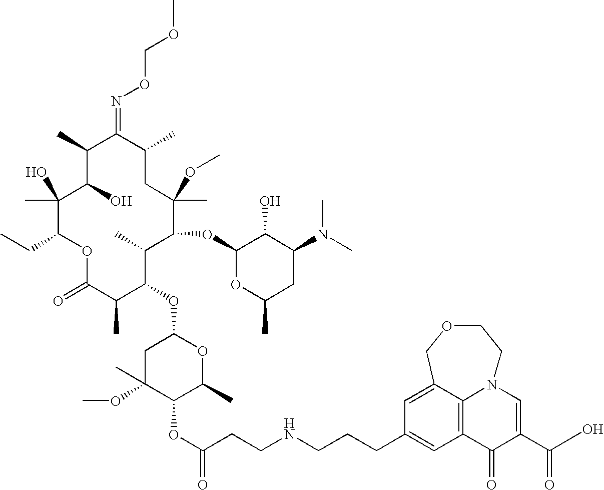

Intermediate 15 (0.105 g, 0.25 mmol), Intermediate 11 (0.170 g, 0.2 mmol) and triethylamine (0.1 mL, 0.7 mmol) in DMSO (0.8 mL) were heated at 80° C. After 16 h the reaction mixture was concentrated and chromatographed over silica gel eluting with 0-15% (9:1 MeOH/0.880M ammonia) in dichloromethane to yield the title compound (0.1 g) as a white solid; ESMS m/z 1149.8 [M+H]⁺.

### Example 28

### 4″-O-{3-[2-{[7-carboxy-8-oxo-3,4-dihydro-2H,8H-[1,4]oxazepino[2,3,4-ij]quinolin-10-yl]thio}ethylamino]propionyl}-erythromycin A (9E)-methoxymethyloxime

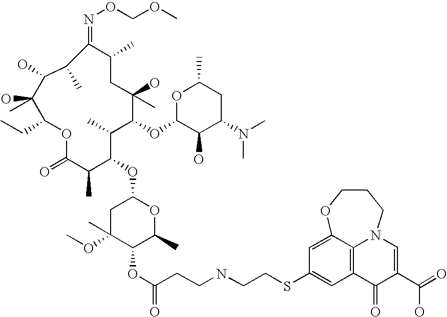

Using a similar procedure to that described in Example 25, Intermediate 11 (0.261 g, 0.308 mmol) and Intermediate 16 (0.187 g, 0.431 mmol) gave the title compound as a white solid (0.028 g). ESMS m/z 1168.2 [M+H]⁺.

### Example 29

### 4″-O-{3-[2-{[7-carboxy-8-oxo-3,4-dihydro-2H,8H-[1,4]oxazepino[2,3,4-ij]quinolin-10-yl]thio}ethylamino]propionyl}-erythromycin A (9E)-oxime

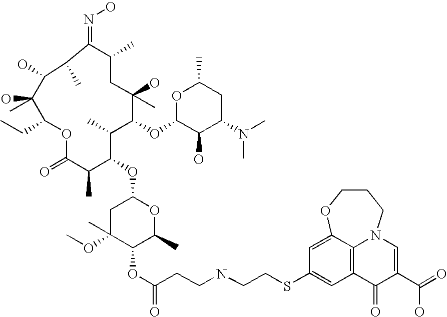

Using a similar procedure to that described in Example 25, Intermediate 6 (0.32 g, 0.402 mmol) and Intermediate 16 (0.20 g, 0.563 mmol) gave the title compound as a white solid (0.028 g). ESMS m/z 1124.0 [M+H]⁺.

### Example 30

### 4″-O-{2-[2-(6-Carboxy-3,3-dimethyl-7-oxo-1H,7H-2-[1,3]oxazino[5,4,3-ij]quinolin-9-yl)thio)ethoxy]ethylcarbamoyl}-erythromycin A 9(E)-(2-diethylaminoethyl)oxime

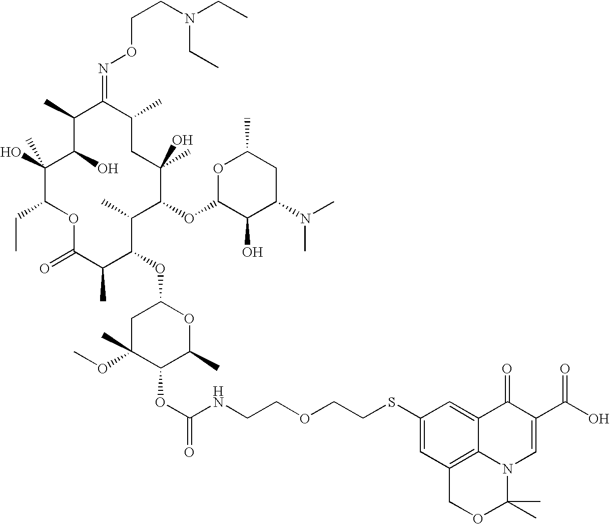

### a) 2′-O-Acetyl-4″-O-{2-[2-(6-carboxy-3,3-dimethyl-7-oxo-1H,7H-2-[1,3]oxazino[5,4,3-ij]quinolin-9-yl)thio)ethoxy]ethylcarbamoyl}-erythromycin A 9(E)-(2-diethylaminoethyl)oxime

Intermediate 20 (0.30 g) was dissolved in dry DMF (5 mL) under nitrogen. To this solution a solution of Intermediate 17 (0.15 g) and DBU (0.230 mL) in dry DMF (5 mL) was added dropwise at room temperature. The resulting mixture was stirred at room temperature for 72 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 5-10% of 9/1 methanol/0.880 ammonia in dichloromethane) to give the title compound (0.170 g) as white foam; ESMS m/z 1294.2 [M+H]⁺.

### b) 4″-O-{2-[2-(6-Carboxy-3,3-dimethyl-7-oxo-1H,7H-2-[1,3]oxazino[5,4,3-ij]quinolin-9-yl)thio)ethoxy]ethylcarbamoyl}-erythromycin A 9(E)-(2-diethylaminoethyl)oxime

A solution of Example 30a (0.170 g) in MeOH (5 mL) was stirred at 50° C. overnight. The solvent was removed in vacuo and the residue was purified by preparative reverse phase HPLC to obtain the title compound as trifluoroacetate salt. The residue was converted to the free base on a silica gel cartridge (eluent 10% of 9/1 methanol/0.880 ammonia in dichloromethane) to give the title compound (0.120 g); ESMS m/z 1257.7 [M+H]⁺.

### Example 31

### 4″-O-{2-[3-(6-Carboxy-3,3-dimethyl-7-oxo-1H,7H-2-[1,3]oxazino[5,4,3-ij]quinolin-9-yl)propyloxy]ethylcarbamoyl}-erythromycin A 9(E)-cyanomethyloxime

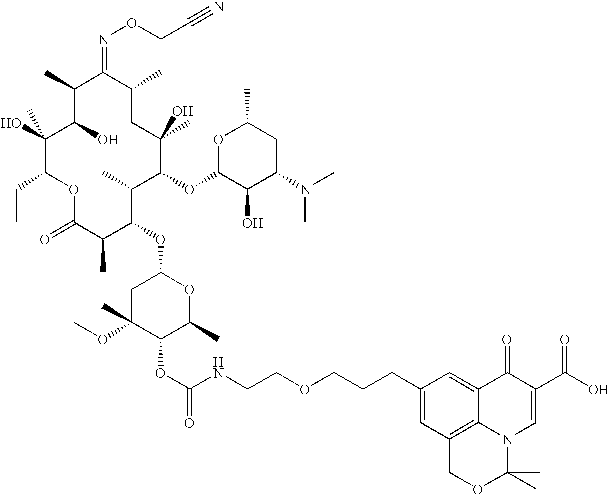

### a) 2′-O-Acetyl-4″-O-{2-[3-(6-Carboxy-3,3-dimethyl-7-oxo-1H,7H-2-[1,3]oxazino[5,4,3-ij]quinolin-9-yl)propyloxy]ethylcarbamoyl}-erythromycin A 9(E)-cyanomethyloxime

To a solution of Intermediate 24 (0.300 g) in dry DMF (4 mL) a solution of Intermediate 18 (0.139 g) and DBU (0.146 mL) in dry DMF (4 mL) was added dropwise at room temperature. The resulting mixture was stirred at 40° C. for 20 h. The crude product was purified using reverse phase chromatography (10 g C18 silica gel, reaction mixture charged as such on the cartridge, eluent 150 mL water/acetonitrile 95/5 with 0.5% formic acid; then 100 mL water/acetonitrile 5/95 with 0.5% formic acid); the acetonitrile fraction was evaporated in vacuo and the resulting residue was purified by flash chromatography (silica gel, eluent 0-10% of 9/1 methanol/0.880 ammonia in dichloromethane) to give the title compound (0.110 g) as white foam; ESMS m/z 1216.5 [M+H]⁺.

### b) 4″-O-{2-[3-(6-Carboxy-3,3-dimethyl-7-oxo-1H,7H-2-[1,3]oxazino[5,4,3-ij]quinolin-9-yl)propyloxy]ethylcarbamoyl}-erythromycin A 9(E)-cyanomethyl oxime

A solution of Example 31a (0.110 g) in MeOH (5 mL) was stirred at 50° C. overnight. The solvent was removed in vacuo and the residue was purified by preparative reverse phase HPLC to obtain the title compound as trifluoroacetate salt; the residue was converted to the free base on silica gel cartridge (eluent 10% of 9/1 methanol/0.880 ammonia in dichloromethane) to give the title compound (0.065 g); ESMS m/z 1174.9 [M+H]⁺.

### Example 32

### 4″-O-{2-[3-(6-Carboxy-7-oxo-1H,7H-2-[1,3]oxazino[5,4,3-ij]quinolin-9-yl)propyloxy]ethylcarbamoyl}-erythromycin A 9(E)-(diethylaminoethyl) oxime

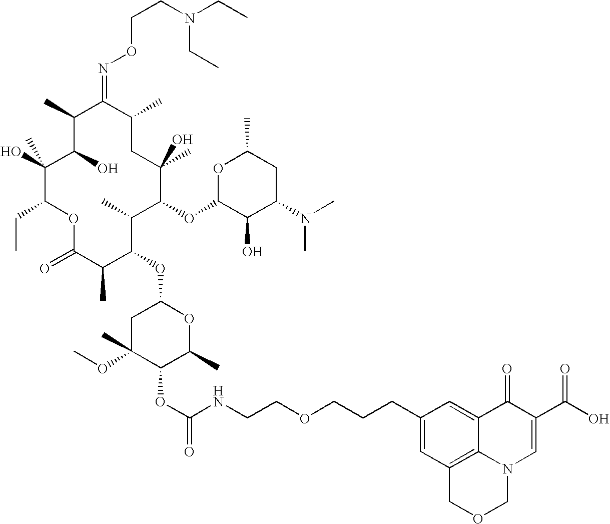

### a) 2′-O-Acetyl-4″-O-{2-[3-(6-carboxy-7-oxo-1H,7H-2-[1,3]oxazino[5,4,3-ij]quinolin-9-yl)propyloxy]ethylcarbamoyl}-erythromycin A 9(E)-(diethylamino ethyl)oxime

To a solution of Intermediate 20 (0.300 g) in dry DMF (4 mL) a solution of Intermediate 19 (0.123 g) and DBU (0.135 mL) in dry DMF (4 mL) was added dropwise at room temperature. The resulting mixture was stirred at 40° C. for 20 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid); the acetonitrile fraction was evaporated in vacuo and the resulting residue was purified by flash chromatography (silica gel, eluent 0-10% of 9/1 methanol/20M ammonia in dichloromethane) to give the title compound (0.150 g) as white foam; ESMS m/z 1248.3 [M+H]⁺.

### b) 4″-O-{2-[3-(6-Carboxy-7-oxo-1H,7H-2-[1,3]oxazino[5,4,3-ij]quinolin-9-yl)propyloxy]ethylcarbamoyl}-erythromycin A 9(E)-(diethylaminoethyl)oxime

A solution of Example 32a (0.167 g) in MeOH (5 mL) was stirred at 50° C. overnight. The solvent was removed in vacuo to give the title compound (0.152 g); ESMS m/z 1207.0 [M+H]⁺.

### Example 33

### 4″-O-{2-[3-(6-Carboxy-7-oxo-1H,7H-2-[1,3]oxazino[5,4,3-ij]quinolin-9-yl)propyloxy]ethylcarbamoyl}-erythromycin A 9(E)-methoxymethyloxime

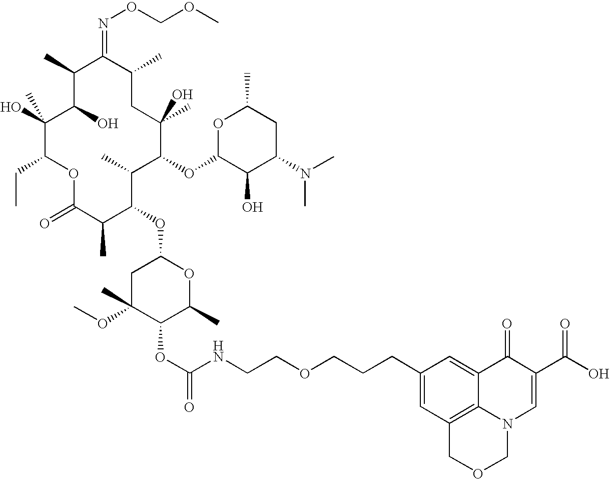

### a) 2′-O-Acetyl-4″-O-{2-[3-(6-Carboxy-7-oxo-1H,7H-2-[1,3]oxazino[5,4,3-ij]quinolin-9-yl)propyloxy]ethylcarbamoyl}-erythromycin A 9(E)-methoxymethyl oxime

To a solution of Intermediate 21 (0.300 g) in dry DMF (3 mL) a solution of Intermediate 19 (0.130 g) and DBU (0.145 mL) in dry DMF (3 mL) was added dropwise at room temperature. The resulting mixture was stirred at 40° C. for 20 h. Solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 5-10% of 9/1 methanol/0.880M ammonia in dichloromethane) to give the title compound (0.180 g) as white foam; ESMS m/z 1193.3 [M+H]⁺.

### b) 4″-O-{2-[3-(6-Carboxy-7-oxo-1H,7H-2-[1,3]oxazino[5,4,3-ij]quinolin-9-yl)propyloxy]ethylcarbamoyl}-erythromycin A 9(E)-methoxymethyloxime

A solution of Example 33a (0.180 g) in MeOH (5 mL) was stirred at 50° C. overnight. The solvent was removed in vacuo to give the title compound (0.165 g); ESMS m/z 1151.8 [M+H]⁺.

### Example 34

### 4″-O-{2-[3-(6-Carboxy-7-oxo-1H,7H-2-[1,3]oxazino[5,4,3-ij]quinolin-9-yl)propyloxy]ethylcarbamoyl}-erythromycin A 9(E)-methyloxime

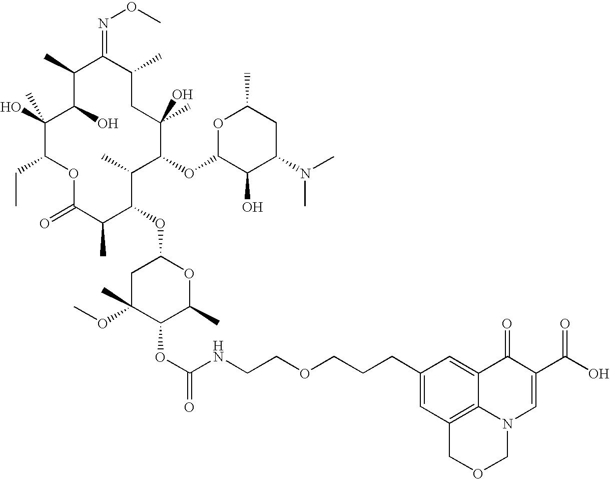

### a) 2′-O-Acetyl-4″-O-{2-[3-(6-carboxy-7-oxo-1H,7H-2-[1,3]oxazino[5,4,3-ij]quinolin-9-yl)propyloxy]ethylcarbamoyl}-erythromycin A 9(E)-methyloxime

To a solution of Intermediate 22 (0.300 g) in dry DMF (3 mL) a solution of Intermediate 19 (0.134 g) and DBU (0.150 mL) in dry DMF (3 mL) was added dropwise at room temperature. The resulting mixture was stirred at 40° C. for 20 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid); the acetonitrile fraction was evaporated in vacuo and the resulting residue was purified by flash chromatography (silica gel, eluent 5-10% of 9/1 methanol/0.880M ammonia in dichloromethane) to give the title compound (0.143 g) as white foam; ESMS m/z 1177.2 [M+H]⁺.

### b) 4″-O-{2-[3-(6-carboxy-7-oxo-1H,7H-2-[1,3]oxazino[5,4,3-ij]quinolin-9-yl)propyloxy]ethylcarbamoyl}-erythromycin A 9(E)-methyloxime

A solution of Example 34a (0.143 g) in MeOH (5 mL) was stirred at 50° C. overnight. The solvent was removed in vacuo to give the title compound (0.130 g); ESMS m/z 1121.9 [M+H]⁺.

### Example 35

### 4″-O-{2-[2-(6-Carboxy-7-oxo-1H,7H-2-[1,3]oxazino[5,4,3-ij]quinolin-9-yl)-propyloxy]ethylcarbamoyl}-erythromycin A 9(E)-oxime

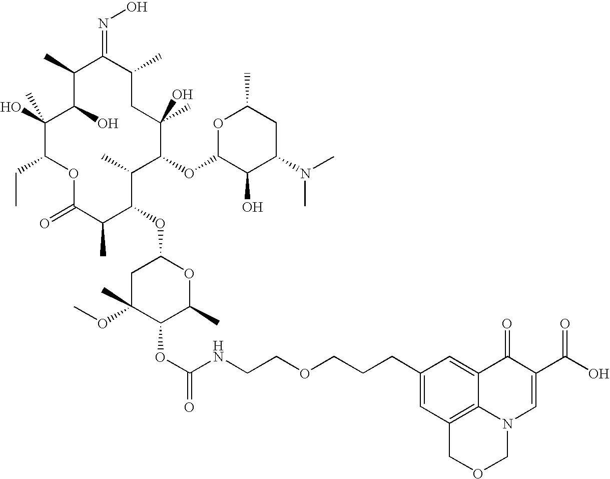

### a) 2′-O-Acetyl-4″-O-{2-[2-(6-Carboxy-7-oxo-1H,7H-2-[1,3]oxazino[5,4,3-ij]quinolin-9-yl)-propyloxy]ethylcarbamoyl}-erythromycin A 9(E)-oxime

To a solution of Intermediate 23 (0.300 g) in dry DMF (4 mL) a solution of Intermediate 19 (0.153 g) and DBU (0.140 mL) in dry DMF (4 mL) was added dropwise at room temperature. The resulting mixture was stirred at 40° C. for 20 h. Solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 0-10% of 9/1 methanol/0.880 ammonia in dichloromethane) to give the title compound (0.130 g) as white foam; ESMS m/z 1149.3 [M+H]⁺.

### b) 4″-O-{2-[2-(6-Carboxy-7-oxo-1H,7H-2-[1,3]oxazino[5,4,3-ij]quinolin-9-yl)-propyloxy]ethylcarbamoyl}-erythromycin A 9(E)-oxime

A solution of Example 35a (0.130 g) in MeOH (5 mL) was stirred at 50° C. overnight. The solvent was removed in vacuo to give the title compound (0.115 g); ESMS m/z 1107.8 [M+H]⁺.

### Example 36

### 4″-O-{2-[3-(6-Carboxy-7-oxo-1H,7H-2-[1,3]oxazino[5,4,3-ij]quinolin-9-yl)propyloxy]ethylcarbamoyl}-erythromycin A 9(E)-(cyanomethyl)oxime

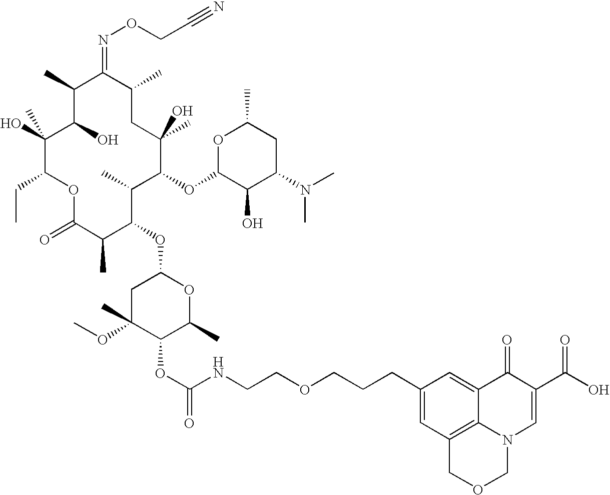

### a) 2′-O-Acetyl-4″-O-{2-[3-(6-carboxy-7-oxo-1H,7H-2-[1,3]oxazino[5,4,3-ij]quinolin-9-yl)propyloxy]ethylcarbamoyl}-erythromycin A 9(E)-(cyanomethyl)oxime

To a solution of Intermediate 24 (0.250 g) in dry DMF (4 mL) a solution of Intermediate 19 (0.100 g) and DBU (0.102 mL) in dry DMF (4 mL) was added dropwise at room temperature. The resulting mixture was stirred at 40° C. for 24 h. The mixture was purified using reverse phase chromatography (10 g C18 silica gel, reaction mixture charged as such on the cartridge, eluent 150 mL water/acetonitrile 95/5 with 0.5% formic acid; then 100 mL water/acetonitrile 5/95 with 0.5% formic acid); the acetonitrile fraction was evaporated in vacuo and the resulting residue was purified by flash chromatography (silica gel, eluent 5-15% of 9/1 methanol/0.880M ammonia in dichloromethane) to give the title compound (0.095 g) as white foam; ESMS m/z 1188.6 [M+H]⁺.

### b) 4″-O-{2-[3-(6-carboxy-7-oxo-1H,7H-2-[1,3]oxazino[5,4,3-ij]quinolin-9-yl)propyloxy]ethylcarbamoyl}-erythromycin A 9(E)-(cyanomethyl)oxime

A solution of Example 36a (0.095 g) in MeOH (5 mL) was stirred at 50° C. overnight. The solvent was removed in vacuo to give the title compound (0.092 g); ESMS m/z 1146.8 [M+H]⁺.

### Example 37

### 4″-O-{2-[3-(6-Carboxy-3,3-dimethyl-7-oxo-1H,7H-2-[1,3]oxazino[5,4,3-ij]quinolin-9-yl)propyloxy]ethylcarbamoyl}-erythromycin A 9(E)-methoxymethyloxime

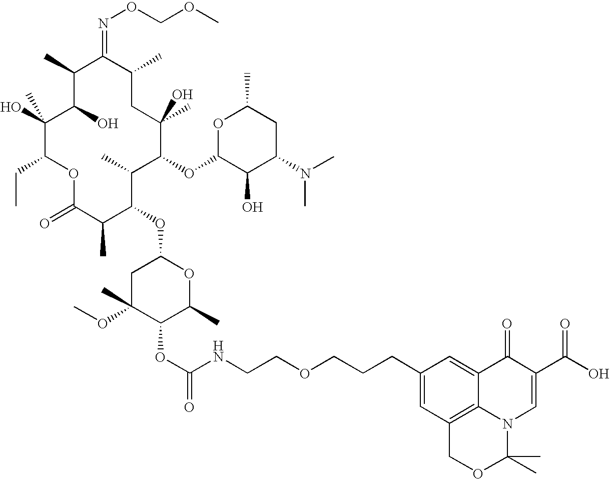

### a) 2′-O-Acetyl-4″-O-{2-[3-(6-Carboxy-3,3-dimethyl-7-oxo-1H,7H-2-[1,3]oxazino[5,4,3-ij]quinolin-9-yl)propyloxy]ethylcarbamoyl}-erythromycin A 9(E)-methoxymethyloxime

To a solution of Intermediate 21 (0.395 g) in dry DMF (6 mL) a solution of Intermediate 18 (0.128 g) and DBU (0.160 mL) in dry DMF (4 mL) was added dropwise at room temperature. The resulting mixture was stirred at room temperature for 72 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid); the acetonitrile fraction was evaporated in vacuo and the resulting residue was purified by flash chromatography (silica gel, eluent 5-10% of 9/1 methanol/0.880M ammonia in dichloromethane) to give the title compound (0.125 g) as white foam; ESMS m/z 1221.6 [M+H]⁺.

### b) 4″-O-{2-[3-(6-Carboxy-3,3-dimethyl-7-oxo-1H,7H-2-[1,3]oxazino[5,4,3-ij]quinolin-9-yl)propyloxy]ethylcarbamoyl}-erythromycin A 9(E)-methoxymethyl oxime

A solution of Example 37a (0.125 g) in MeOH (5 mL) was stirred at 50° C. overnight. The solvent was removed in vacuo to give the title compound (0.112 g); ESMS m/z 1179.9 [M+H]⁺.

### Example 38

### 4″-O-{2-[3-(6-Carboxy-3,3-dimethyl-7-oxo-1H,7H-2-[1,3]oxazino[5,4,3-ij]quinolin-9-yl)propyloxy]ethylcarbamoyl}-erythromycin A 9(E)-methyloxime

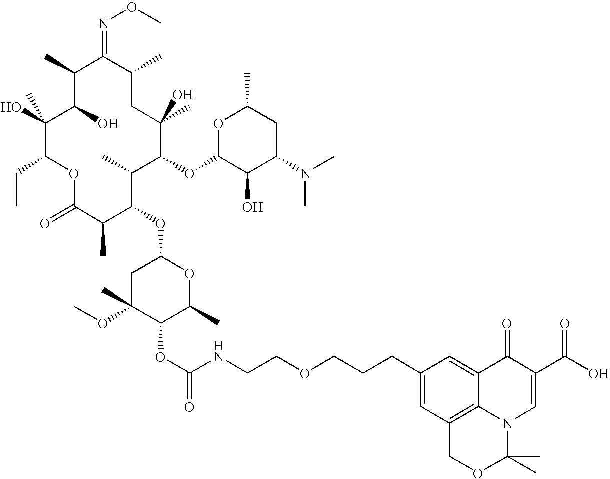

### a) 2′-O-Acetyl-4″-{2-[3-(6-Carboxy-3,3-dimethyl-7-oxo-1H,7H-2-[1,3]oxazino-[5,4,3-ij]quinolin-9-yl)propyloxy]ethylcarbamoyl}-erythromycin A 9(E)-methyloxime

To a solution of Intermediate 22 (0.400 g) in dry DMF (6 mL) a solution of Intermediate 18 (0.133 g) and DBU (0.168 mL) in dry DMF (6 mL) was added dropwise at room temperature. The resulting mixture was stirred at room temperature for 72 h. Solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 5-10% of 9/1 methanol/0.880M ammonia in dichloromethane) to give the title compound (0.177 g) as white foam; ESMS m/z 1191.7 [M+H]⁺.

### b) 4″-O-{2-[3-(6-Carboxy-3,3-dimethyl-7-oxo-1H,7H-2-[1,3]oxazino[5,4,3-ij]quinolin-9-yl)propyloxy]ethylcarbamoyl}-erythromycin A 9(E)-methyloxime

A solution of Example 38a (0.170 g) in MeOH (5 mL) was stirred overnight at 50° C. The solvent was removed in vacuo to give the title compound (0.159 g); ESMS m/z 1150.0 [M+H]⁺.

### Example 39

### 4″-O-{2-[3-(6-Carboxy-3,3-dimethyl-7-oxo-1H,7H-2-[1,3]oxazino[5,4,3-ij]quinolin-9-yl)propyloxy]ethylcarbamoyl}-6-O-methyl-erythromycin A

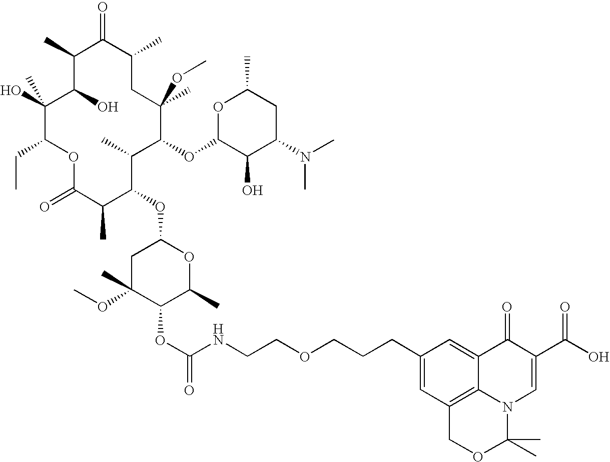

To a solution of Intermediate 27 (0.293 g) in DMF (5 mL) a solution of Intermediate 18 (0.165 g) and DBU (0.157 mL) in DMF (5 mL) was added dropwise at room temperature. The resulting mixture was stirred at 40° C. for 28 h. The solvent was removed in vacuo and the residue was dissolved in MeOH and purified using a reverse phase chromatography (10 g C18 silica gel, eluent 150 mL water/acetonitrile 95/5 with 0.5% formic acid; then 100 mL water/acetonitrile 5/95 with 0.5% formic acid); the acetonitrile fraction was evaporated in vacuo and the resulting residue was purified by flash chromatography (silica gel, eluent 2-8% of 9/1 methanol/0.880M ammonia in dichloromethane) to give the title compound (0.145 g); ESMS m/z 1134.7 [M+H]⁺.

### Example 40

### 4″-O-{2-[3-(6-Carboxy-3,3-dimethyl-7-oxo-1H,7H-2-[1,3]oxazino[5,4,3-ij]quinolin-9-yl)propyloxy]ethylcarbamoyl}-erythromycin A 9(E)-(2-diethylaminoethyl)oxime

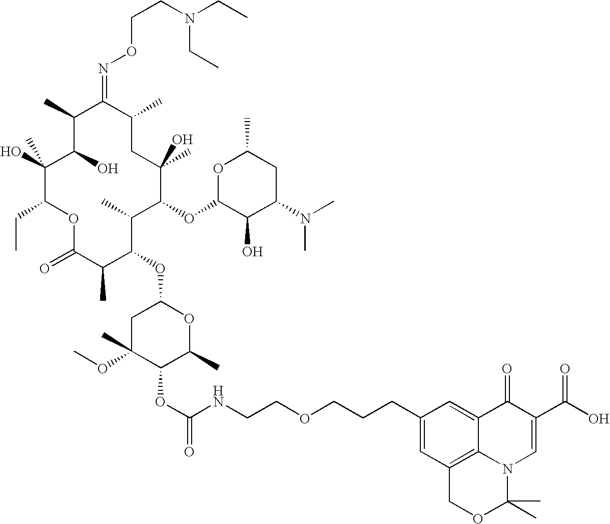

### a) 2′-O-Acetyl-4″-O-{2-[3-(6-Carboxy-3,3-dimethyl-7-oxo-1H,7H-2-[1,3]oxazino[5,4,3-ij]quinolin-9-yl)propyloxy]ethylcarbamoyl}-erythromycin A 9(E)-(2-diethylaminoethyl)oxime

To a solution of Intermediate 20 (0.300 g) in dry DMF (5 mL) a solution of Intermediate 18 (0.145 g) and DBU (0.138 mL) in dry DMF (6 mL) was added dropwise at room temperature. The resulting mixture was stirred at room temperature for 72 h. Solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid); the acetonitrile fraction was evaporated in vacuo and the resulting residue was purified by flash chromatography (silica gel, eluent 5-10% of 9/1 methanol/0.880M ammonia in dichloromethane) to give the title compound (0.205 g) as white foam; ESMS m/z 1276.7 [M+H]⁺.

### b) 4″-O-{2-[3-(6-Carboxy-3,3-dimethyl-7-oxo-1H,7H-2-[1,3]oxazino[5,4,3-ij]quinolin-9-yl)propyloxy]ethylcarbamoyl}-erythromycin A 9(E)-(2-diethylamino ethyl)oxime

A solution of Example 40a (0.205 g) in MeOH (8 mL) was stirred at 50° C. overnight. The solvent was removed in vacuo to give the title compound (0.184 g); ESMS m/z 1234.5 [M+H]⁺.

### Example 41

### 4″-O-{2-[3-(6-Carboxy-3,3-dimethyl-7-oxo-1H,7H-2-[1,3]oxazino[5,4,3-ij]quinolin-9-yl)propyloxy]ethylcarbamoyl}-erythromycin A 9(E)-oxime

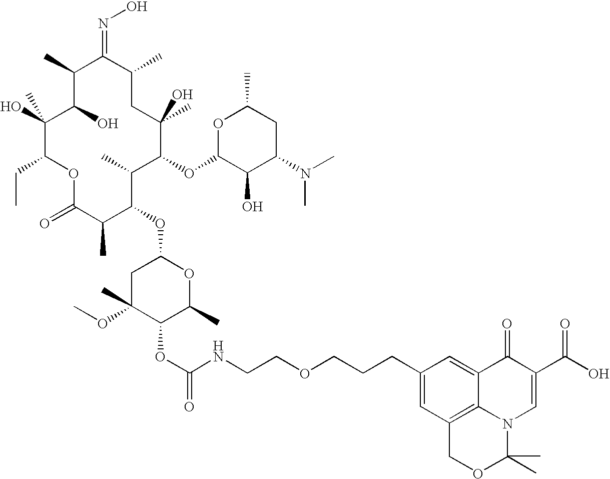

### a) 2′-O-Acetyl-4″-O-{2-[3-(6-Carboxy-3,3-dimethyl-7-oxo-1H,7H-2-[1,3]oxazino-[5,4,3-ij]quinolin-9-yl)propyloxy]ethylcarbamoyl}-erythromycin A 9(E)-oxime

To a solution of Intermediate 25 (0.384 g) in dry DMF (6 mL) a solution of Intermediate 18 (0.187 g) and DBU (0.178 mL) in dry DMF (6 mL) was added dropwise at room temperature. The resulting mixture was stirred at room temperature for 72 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 5-10% of 9/1 methanol/0.880M ammonia in dichloromethane) to give the title compound (0.309 g) as white foam; ESMS m/z 1267.5 [M+H]⁺.

### b) 4″-O-{2-[3-(6-Carboxy-3,3-dimethyl-7-oxo-1H,7H-2-[1,3]oxazino[5,4,3-ij]quinolin-9-yl)propyloxy]ethylcarbamoyl}-erythromycin A 9(E)-oxime

Intermediate 41a (0.309 g) was dissolved in MeOH (24 mL) and to this solution ammonium formate (0.245 g) and 10% palladium on carbon (0.122 g) were added. The resulting mixture was heated under reflux under nitrogen for 7 h. Catalyst was filtered off and the solvent removed in vacuo. The residue was purified by flash chromatography (silica gel, eluent 5-12% of 9/1 methanol/0.880 ammonia in dichloromethane) to give the title compound (0.075 g) as white foam; ESMS m/z 1135.6 [M+H]⁺.

### Example 42

### 4″-O-{2-[3-(6-Carboxy-3,3-dimethyl-7-oxo-1H,7H-2-[1,3]oxazino[5,4,3-ij]quinolin-9-yl-propyloxy]-ethylcarbamoyl}-(9S)-9-O-11-O-ethylidene 9-dihydroerythromycin A

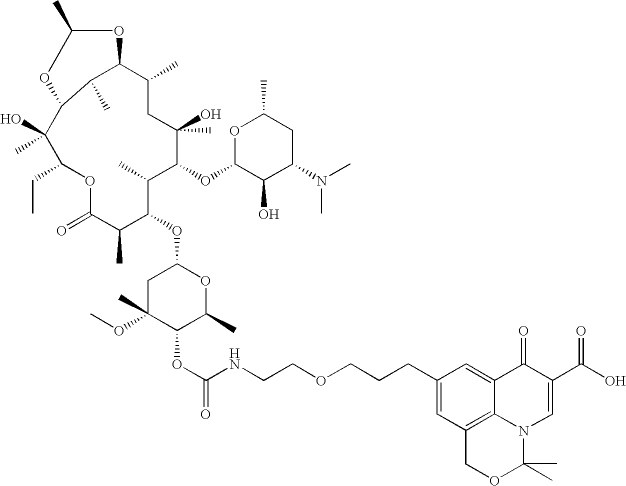

### a) 2′-O-Acetyl-4″-O-{2-[3-(6-Carboxy-3,3-dimethyl-7-oxo-1H,7H-2-[1,3]oxazino[5,4,3-j]quinolin-9-yl-propyloxy]-ethylcarbamoyl}-(9S)-9-O,11-O-ethylidene 9-dihydroerythromycin A

To a solution of Intermediate 26 (0.300 g) in dry DMF (5 mL) a solution of Intermediate 18 (0.158 g) and DBU (0.151 mL) in dry DMF (5 mL) was added dropwise at room temperature. The resulting mixture was stirred at room temperature for 72 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid); the acetonitrile fraction was evaporated in vacuo and the resulting residue was purified by flash chromatography (silica gel, eluent 5-10% of 9/1 methanol/0.880M ammonia in dichloromethane) to give the title compound (0.201 g) as white foam; ESMS m/z 1190.5 [M+H]⁺.

### b) 4″-O-{2-[3-(6-Carboxy-3,3-dimethyl-7-oxo-1H,7H-2-[1,3]oxazino[5,4,3-ij]quinolin-9-yl-propyloxy]-ethylcarbamoyl}-19S)-9-O,11-O-ethylidene 9-dihydro erythromycin A

A solution of Example 42a (0.201 g) in MeOH (8 mL) was stirred at 50° C. overnight. The solvent was removed in vacuo to give the title compound (0.182 g); ESMS m/z 1148.7 [M+H]⁺.

### Example 43

### 4″-O-{2-[2-(6-Carboxy-3,3-dimethyl-7-oxo-1H,7H-2-[1,3]oxazino[5,4,3-ij]quinolin-9-yl)thio)ethoxy]ethylcarbamoyl}-erythromycin A 9(E)-oxime trifluoroacetic acid salt

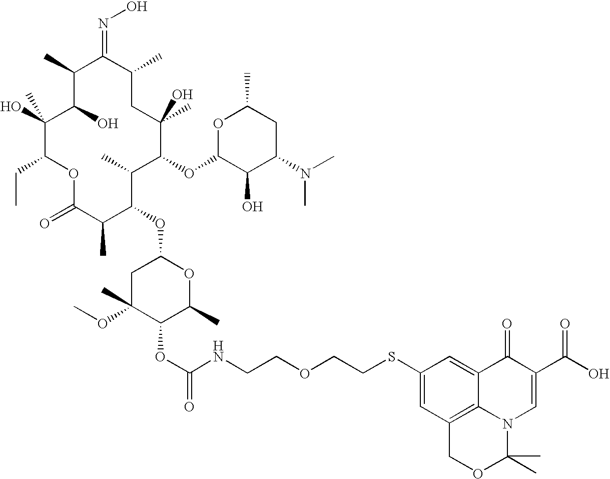

### a) 2′-O-Acetyl-4″-O-{2-[2-(6-Carboxy-3,3-dimethyl-7-oxo-1H,7H-2-[1,3]oxazino-[5,4,3-ij]quinolin-9-yl)thio)ethoxy]ethylcarbamoyl}-erythromycin A 9(E)-oxime

To a solution of Intermediate 23 (0.239 g) in dry DMF (5 mL) a solution of Intermediate 17 (0.123 g) and DBU (0.150 mL) in dry DMF (5 mL) was added dropwise at room temperature. The resulting mixture was stirred at room temperature for 72 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 5-13% of 9/1 methanol/20M ammonia in DCM) to give the title compound (0.113 g) as white foam; ESMS m/z 1195.2 [M+H]⁺.

### b) 4″-O-{2-[2-(5-Carboxy-3,3-dimethyl-6-oxo-1H,6H-2-oxa-3a-aza-phenalen-8-sulfanyl)-ethoxy]-ethylcarbamoyl}-erythromycin A 9(E)-oxime TFA salt

A solution of Example 43a (0.113 g) in MeOH (6 mL) was stirred at 50° C. overnight. The solvent was removed in vacuo. The residue was purified by preparative reverse phase HPLC to obtain the title compound (0.068 g); ESMS m/z 1153.5 [M+H]⁺.

### Example 44

### 4″-O-{2-[3-(6-Carboxy-3,3-dimethyl-7-oxo-1H,7H-2-[1,3]oxazino[5,4,3-ij]quinolin-9-yl)thio)-ethoxy]-ethylcarbamoyl}-erythromycin A 9(E)-methoxymethyloxime trifluoroacetic acid salt

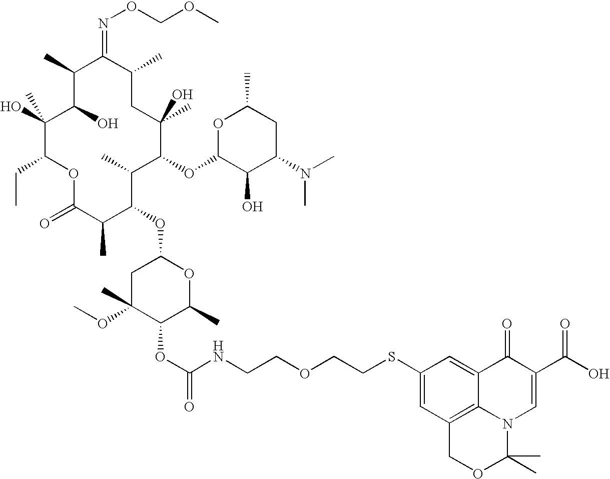

### a) 2′-O-Acetyl-4″-O-{2-[3-(6-Carboxy-3,3-dimethyl-7-oxo-1H,7H-2-[1,3]oxazino[5,4,3-ij]quinolin-9-yl)thio)-ethoxy]-ethylcarbamoyl}-erythromycin A 9(E)-methoxymethyloxime

To a solution of Intermediate 21 (0.232 g) in dry DMF (5 mL) a solution of Intermediate 17 (0.123 g) and DBU (0.150 mL) in dry DMF (5 mL) was added dropwise at room temperature. The resulting mixture was stirred at room temperature for 72 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 5-10% of 9/1 methanol/0.880M ammonia in DCM) to give the title compound (0.098 g) as white foam; ESMS m/z 1239.3 [M+H]⁺.

### b) 4″-O-{2-[3-(6-Carboxy-3,3-dimethyl-7-oxo-1H,7H-2-[1,3]oxazino[5,4,3-ij]quinolin-9-yl)thio)-ethoxy]-ethylcarbamoyl}-erythromycin A 9-methoxymethyl oxime TFA salt

A solution of Example 44a (0.113 g) in MeOH (6 mL) was stirred at 50° C. overnight. The solvent was removed in vacuo. The residue was purified by preparative reverse phase HPLC to obtain the title compound (0.055 g); ESMS m/z 1197.5 [M+H]⁺.

Biological Data

Using a standard broth dilution method in microtitre, compounds were tested for antibacterial activity. The compounds in the above examples gave minimum inhibitory concentrations (MICs) less than 1 microgram per millilitre against erythromycin-sensitive and erythromycin-resistant strains of Streptococcus pneumoniae and Streptococcus pyogenes.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps. The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A compound of formula (I) 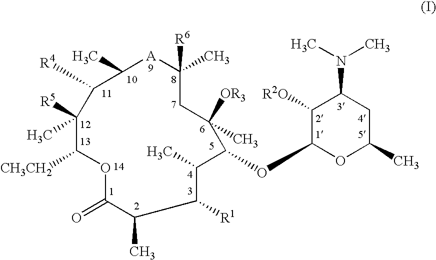
wherein
A is a bivalent radical —C(O)—, —N(R⁷)—CH₂—, —CH(NR⁸R⁹)— or —C(═NR¹⁰)—, or A and R⁴ taken together with the intervening atoms form a cyclic group having the following formula: 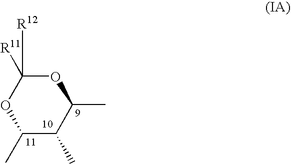
and R¹ is a group having the following formula: 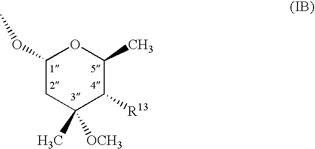
wherein R¹³ is —OC(O)(CH₂)_{d}U¹R¹⁴, —OC(O)N(R¹⁵)(CH₂)_{d}U¹R¹⁴, —O(CH₂)_{d}U¹R¹⁴, 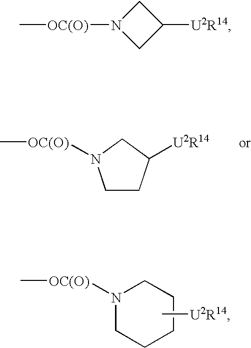
or
A is the bivalent radical —N(R⁷)—CH₂— and R¹ is a group having the following formula: 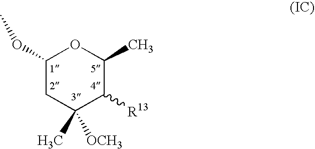
wherein R¹³ is —NHC(O)(CH₂)_{d}U¹R¹⁴;
R² is hydrogen or a hydroxyl protecting group;
R³ is hydrogen, C₁₋₄alkyl, or C₃₋₆alkenyl optionally substituted by 9- or 10-membered fused bicyclic heteroaryl;
R⁴ is hydroxy, C₃₋₆alkenyloxy optionally substituted by 9- or 10-membered fused bicyclic heteroaryl, or C₁₋₆alkoxy optionally substituted by C₁₋₆alkoxy or —O(CH₂)ₑNR⁷R¹⁶, or R⁴ and A taken together with the intervening atoms form a cyclic group of formula (IA),
R⁵ is hydroxy, or
R⁴ and R⁵ taken together with the intervening atoms form a cyclic group having the following formula: 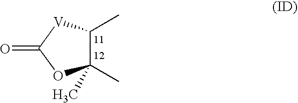
wherein V is a bivalent radical —CH₂—, —CH(CN)—, —O—, —N(R¹⁷)— or —CH(SR¹⁷)—, with the proviso that when R¹ is a group of formula (IC), V is —O—;
R⁶ is hydrogen or fluorine;
R⁷ is hydrogen or C₁₋₆alkyl;
R⁸ and R⁹ are each independently hydrogen, C₁₋₆alkyl or —C(O)R¹⁸, or
R⁸ and R⁹ together form ═CH(CR¹⁸R¹⁹)_{f}aryl, ═CH(CR¹⁸R¹⁹)_{f}heterocyclyl, ═CR¹⁸R¹⁹ or ═C(R¹⁸)C(O)OR¹⁸, wherein the alkyl, aryl and heterocyclyl groups are optionally substituted by up to three groups independently selected from R²⁰;
R¹⁰ is —OR²¹;
R¹¹ and R¹² are each independently hydrogen, C₁₋₆alkyl, heteroaryl, or aryl optionally substituted by one or two groups independently selected from hydroxyl and C₁₋₆alkoxy;
R¹⁴ is a heterocyclic group having the following formula: 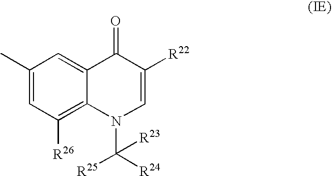
R¹⁵, R¹⁶, R¹⁸ and R¹⁹ are each independently hydrogen or C₁₋₆alkyl;
R¹⁷ is hydrogen or C₁₋₄alkyl optionally substituted by a group selected from optionally substituted phenyl, optionally substituted 5- or 6-membered heteroaryl and optionally substituted 9- or 10-membered fused bicyclic heteroaryl;
R²⁰ is halogen, cyano, nitro, trifluoromethyl, azido, —C(O)R²⁷, —C(O)OR²⁷, —OC(O)R²⁷—OC(O)OR²⁷, —NR²⁸C(O)R²⁹, —C(O)NR²⁸R²⁹, —NR²⁸R²⁹, hydroxy, C₁₋₆alkyl, —S(O)_{g}C₁₋₆alkyl, C₁₋₆alkoxy, —(CH₂)ₕaryl or —(CH₂)ₕheteroaryl, wherein the alkoxy group is optionally substituted by up to three groups independently selected from —NR¹⁸R¹⁹ halogen and
—OR¹⁸, and the aryl and heteroaryl groups are optionally substituted by up to five groups independently selected from halogen, cyano, nitro, trifluoromethyl, azido, —C(O)R³⁰,
—C(O)OR³⁰, —OC(O)OR³⁰, —NR³¹C(O)R³², —C(O)NR³¹R³²NR³¹R³², hydroxy, C₁₋₆alkyl and C₁₋₆alkoxy;
R²¹ is hydrogen, C₁₋₆alkyl, C₃₋₇cycloalkyl, C₃₋₆alkenyl or a 5- or 6-membered heterocyclic group, wherein the alkyl, cycloalkyl, alkenyl and heterocyclic groups are optionally substituted by up to three groups independently selected from optionally substituted 5- or 6-membered heterocyclic group, optionally substituted 5- or 6-membered heteroaryl,
—OR³³, —S(O)ᵢR³³, —NR³³R³⁴, —CONR³³R³⁴, halogen and cyano;
R²² is —C(O)OR³⁵, —C(O)NHR³⁵, —C(O)CH₂NO₂ or —C(O)CH₂SO₂R⁷;
R²³ and R²⁴ are each independently hydrogen or methyl;
R²⁵ and R²⁶ are linked to form a bivalent radical —OCH₂—, —CH₂O—, —O(CH₂)₂—, —CH₂OCH₂— or —(CH₂)₂O—;
R²⁷ is hydrogen, C₁₋₁₀alkyl, —(CH₂)ⱼaryl or —(CH₂)ⱼheteroaryl;
R²⁸ and R²⁹ are each independently hydrogen, —OR¹⁸, C₁₋₆alkyl, —(CH₂)ₖaryl or —(CH₂)ₖheterocyclyl;
R³⁰ is hydrogen, C₁₋₁₀alkyl, —(CH₂)ₘaryl or —(CH₂)ₘheteroaryl;
R³¹ and R³² are each independently hydrogen, —OR¹⁸, C₁₋₆alkyl, —(CH₂)ₙaryl or —CH₂)ₙheterocyclyl;
R³³ and R³⁴ are each independently hydrogen, C₁₋₄alkyl or C₁₋₄alkoxyC₁₋₄alkyl;
R³⁵ is hydrogen,
C₁₋₆alkyl optionally substituted by up to three groups independently selected from halogen, cyano, C₁₋₄alkoxy optionally substituted by phenyl or C₁₋₄alkoxy, —C(O)C₁₋₆alkyl, —C(O)OC₁₋₆alkyl, —OC(O)C₁₋₆alkyl, —OC(O)OC₁₋₆alkyl, —C(O)NR³⁶R³⁷,
—NR³⁶R³⁷ and phenyl optionally substituted by nitro or —C(O)OC₁₋₆alkyl,
—(CH₂)ₚC₃₋₇cycloalkyl,
—(CH₂)ₚheterocyclyl,
—(CH₂)ₚheteroaryl,
—(CH₂)ₚaryl,
C₃₋₆alkenyl, or
C₃₋₆alkynyl;
R³⁶ and R³⁷ are each independently hydrogen or C₁₋₆alkyl optionally substituted by phenyl or —C(O)OC₁₋₆alkyl, or
R³⁶ and R³⁷, together with the nitrogen atom to which they are bound, form a 5- or 6-membered heterocyclic group optionally containing one additional heteroatom selected from oxygen, sulfur and N—R³⁸;
R³⁸ is hydrogen or methyl;
R³⁹ is hydrogen, C₁₋₄alkyl, C₃₋₇cycloalkyl, optionally substituted phenyl or benzyl, acetyl or benzoyl;
U¹ is a bivalent radical —W(CH₂)_{q}X—, W(CH₂)_{q}—, —W(CH₂)_{q}X(CH₂)ᵣY—, —W(CH₂)_{q}X(CH₂)ᵣ—,
—W(CH₂)_{q}X(CH₂)ᵣY(CH₂)ₛZ- or —W(CH₂)_{q}X(CH₂)ᵣY(CH₂)ₛ—;
U² is U¹ or a bivalent radical —O—, —N(R³⁹)—, —S(O)ₜ— or —CH₂—;
W, X, Y and Z are each independently a bivalent radical —N(R³⁹)—, —O—, —S(O)ₜ—, —N(R³⁹)C(O)—, —C(O)N(R³⁹)— or —N[C(O)R³⁹]—;
d is an integer from 2 to 5;
e is an integer from 2 to 4;
f, h, j, k, m, n and p are each independently integers from 0 to 4;
g, i and t are each independently integers from 0 to 2; and
q, r and s are each independently integers from 2 to 5;
with the proviso that when R²³ and R²⁴ are each hydrogen, R²⁵ and R²⁶ are not linked to form the bivalent radical —CH₂O—, and
when R²³ and R²⁴ are each hydrogen and U¹ is —W(CH₂)_{q}X(CH₂)ᵣY—, —W(CH₂)_{q}X(CH₂)ᵣ—,
—W(CH₂)_{q}X(CH₂)ᵣY(CH₂)ₛZ- or —W(CH₂)_{q}X(CH₂)ᵣY(CH₂)ₛ, R²⁵ and R²⁶ are not linked to form the bivalent radical —OCH₂—;
or a pharmaceutically acceptable derivative thereof.

2. A compound according to claim 1 wherein A is —C(O)—, —N(R⁷)—CH₂— or —C(═NR¹⁰)—.

3. A compound according to claim 1 wherein R¹ is

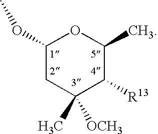

4. A compound according to claim 1 wherein U¹ is —W(CH₂)_{q}—.

5. A compound according to claim 1 wherein q is 3.

6. A compound according to claim 1 wherein R¹⁴ is a heterocyclic group having one of the following formulae:

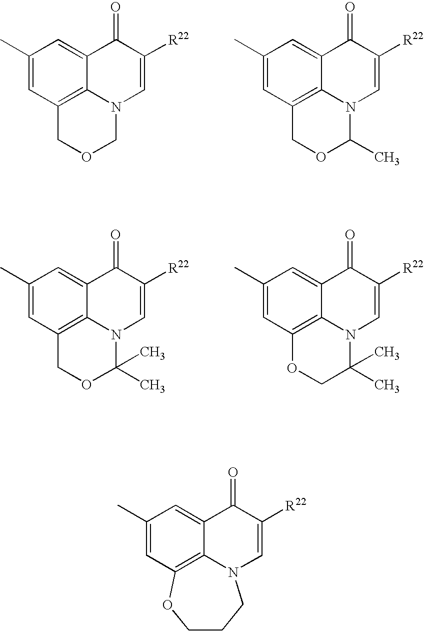

7. A compound according to claim 1 as defined in any one of Examples 1 to 21, or a pharmaceutically acceptable derivative thereof.

8. A compound selected from:
4″-O-{[(2-{[3-(6-carboxy-3-(R,S)-methyl-7-oxo-1H,7H-[1,3]oxazino[5,4,3-ij]quinolin-9-yl)propyl]amino}propionyl]-6-O-methyl erythromycin A,
4″-O-{[(2-{[3-(6-carboxy-3-(R,S)-methyl-7-oxo-1H,7H-[1,3]oxazino[5,4,3-ij]quinolin-9-yl)propyl]amino}propionyl]-azithromycin,
4″-O-{[(2-{[3-(6-carboxy-3-(R,S)-methyl-7-oxo-1H,7H-[1,3]oxazino[5,4,3-ij]quinolin-9-yl)propyl]amino}propionyl]-azithromycin-11,12-carbonate,
4″-O-[(2-{[3-(6-carboxy-3,3-dimethyl-7-oxo-1H,7H-[1,3]oxazino[5,4,3-ij]quinolin-9-yl)propyl]amino}propionyl]-azithromycin,
4″-O-[(2-{[3-(6-carboxy-3,3-dimethyl-7-oxo-1H,7H-[1,3]oxazino[5,4,3-ij]quinolin-9-yl)propyl]amino}propionyl]-6-O-methyl erythromycin A,
4″-O-[(2-{[3-(6-carboxy-3,3-dimethyl-7-oxo-1H,7H-[1,3]oxazino[5,4,3-ij]quinolin-9-yl)propyl]amino}propionyl]-azithromycin-11,12-carbonate,
4″-O-[(2-{[3-(6-carboxy-7-oxo-1H,7H-[1,3]oxazino[5,4,3-ij]quinolin-9-yl)propyl]amino}propionyl]-6-O-methyl erythromycin A,
4″-O-[(2-{[3-(6-carboxy-7-oxo-1H,7H-[1,3]oxazino[5,4,3-ij]quinolin-9-yl)propyl]amino}propionyl]-azithromycin,
4″-O-[(2-{[3-(6-carboxy-7-oxo-1H,7H-[1,3]oxazino[5,4,3-ij]quinolin-9-yl)propyl]amino}propionyl]-azithromycin-11,12-carbonate,
4″-O-[(2-{[3-(6-carboxy-3,3-dimethyl-7-oxo-1H,7H-[1,3]oxazino[5,4,3-ij]quinolin-9-yl)propyl]amino}propionyl]-erythromycin A (9E)-oxime,
4″-O-[(2-{[3-(6-carboxy-3,3-dimethyl-7-oxo-1H,7H-[1,3]oxazino[5,4,3-ij]quinolin-9-yl)propyl]amino}propionyl]-erythromycin A (9E)-methoxymethyl oxime,
4″-O-[(2-{[3-(7-carboxy-8-oxo-3,4-dihydro-2H,8H-[1,4]oxazepino[2,3,4-ij]quinolin-10-yl)propyl]amino}propionyl]-6-O-methyl erythromycin A,
4″-O-[(2-{[3-(7-carboxy-8-oxo-3,4-dihydro-2H,8H-[1,4]oxazepino[2,3,4-ij]quinolin-10-yl)propyl]amino}propionyl]-erythromycin (9E)-methoxime,
4″-O-[(2-{[3-(7-carboxy-8-oxo-3,4-dihydro-2H,8H-[1,4]oxazepino[2,3,4-ij]quinolin-10-yl)propyl]amino}propionyl]-azithromycin-11,12-carbonate,
4″-O-[(2-{[3-(7-carboxy-8-oxo-3,4-dihydro-2H,8H-[1,4]oxazepino[2,3,4-ij]quinolin-10-yl)propyl]amino}propionyl]-azithromycin,
4″-O-[(2-{[3-(7-carboxy-8-oxo-3,4-dihydro-2H,8H-[1,4]oxazepino[2,3,4-ij]quinolin-10-yl)propyl]amino}propionyl]-erythromycin (9E)-oxime,
4″-O-[(2-{[3-(7-carboxy-8-oxo-3,4-dihydro-2H,8H-[1,4]oxazepino[2,3,4-ij]quinolin-10-yl)propyl]amino}propionyl]-erythromycin (9E)-methoxymethyl-oxime,
4″-O-[(2-{[3-(6-carboxy-3,3-dimethyl-7-oxo-2,3-dihydro-7H-[1,4]oxazino[2,3,4-ij]quinolin-9-yl)propyl]amino}propionyl]-erythromycin (9E)-methoxymethyl-oxime,
4″-O-[(2-{[3-(6-carboxy-3,3-dimethyl-7-oxo-2,3-dihydro-7H-[1,4]oxazino[2,3,4-ij]quinolin-9-yl)propyl]amino}propionyl]-6-O-methyl erythromycin A,
4″-O-[(2-{[3-(6-carboxy-3,3-dimethyl-7-oxo-2,3-dihydro-7H-[1,4]oxazino[2,3,4-ij]quinolin-9-yl)propyl]amino}propionyl]-erythromycin A (9E)-methoxime,
4″-O-[(2-{[3-(6-carboxy-3,3-dimethyl-7-oxo-2,3-dihydro-7H-[1,4]oxazino[2,3,4-ij]quinolin-9-yl)propyl]amino}propionyl]-azithromycin,
4″-O-{2-[3-(6-carboxy-3,3-dimethyl-7-oxo-1H,7H-2-[1,3]oxazino[5,4,3-ij]quinolin-9-yl)thio)ethoxy]ethylcarbamoyl}-erythromycin A 9(E)-methyloxime trifluoroacetic acid salt,
O-[(2-{[3-(6-carboxy-3,3-dimethyl-7-oxo-1H,7H-[1,3]oxazino[5,4,3-ij]quinolin-9-yl)propyl]methylamino}propionyl]-6-O-methyl erythromycin A,
4″-O-[(2-{[3-(6-carboxy-3,3-dimethyl-7-oxo-1H,7H-[1,3]oxazino[5,4,3-ij]quinolin-9-yl)propyl]methylamino}ethyl]-6-O-methyl erythromycin A,
4″-O {3-[2-{[6-carboxy-7-oxo-2,3-dihydro-7H-[1,4]oxazino[2,3,4-ij]quinolin-9-yl]thio}ethylamino]propionyl}-erythromycin A 9(E)-oxime,
4″-O-[3-{3-(7-carboxy-3,4-dihydro-8-oxo-1H,8H-[1,4]oxazepino[6,5,4,-ij]quinolin-10-yl)propylamino}propionyl]-6-O-methyl erythromycin A,
4″-O-[3-{3-(7-carboxy-3,4-dihydro-8-oxo-1H,8H-[1,4]oxazepino[6,5,4,-ij]quinolin-10-yl)propylamino}propionyl]erythromycin A (9E)-methoxymethyl oxime,
4″-O-{3-[2-{[7-carboxy-8-oxo-3,4-dihydro-2H,8H-[1,4]oxazepino[2,3,4-ij]quinolin-10-yl]thio}ethylamino]propionyl}-erythromycin A (9E)-methoxymethyloxime,
4″-O {3-[2-{[7-carboxy-8-oxo-3,4-dihydro-2H,8H-[1,4]oxazepino[2,3,4-ij]quinolin-10-yl]thio}ethylamino]propionyl}-erythromycin A (9E)-oxime,
4″-O-{2-[2-(6-carboxy-3,3-dimethyl-7-oxo-1H,7H-2-[1,3]oxazino[5,4,3-ij]quinolin-9-yl)thio)ethoxy]ethylcarbamoyl}-erythromycin A 9(E)-(2-diethylaminoethyl)oxime,
4″-O-{2-[3-(6-carboxy-3,3-dimethyl-7-oxo-1H,7H-2-[1,3]oxazino[5,4,3-ij]quinolin-9-yl)propyloxy]ethylcarbamoyl}-erythromycin A 9(E)-cyanomethyloxime,
4″-O-{2-[3-(6-carboxy-7-oxo-1H,7H-2-[1,3]oxazino[5,4,3-ij]quinolin-9-yl)propyloxy]ethylcarbamoyl}-erythromycin A 9(E)-(diethylaminoethyl)oxime,
4″-O-{2-[3-(6-carboxy-7-oxo-1H,7H-2-[1,3]oxazino[5,4,3-ij]quinolin-9-yl)propyloxy]ethylcarbamoyl}-erythromycin A 9(E)-methoxymethyloxime,
4″-O-{2-[3-(6-carboxy-7-oxo-1H,7H-2-[1,3]oxazino[5,4,3-ij]quinolin-9-yl)propyloxy]ethylcarbamoyl}-erythromycin A 9(E)-methyloxime,
4″-O-{2-[2-(6-carboxy-7-oxo-1H,7H-2-[1,3]oxazino[5,4,3-ij]quinolin-9-yl)-propyloxy]ethylcarbamoyl}-erythromycin A 9(E)-oxime,
4″-O-{2-[3-(6-carboxy-7-oxo-1H,7H-2-[1,3]oxazino[5,4,3-ij]quinolin-9-yl)propyloxy]ethylcarbamoyl}-erythromycin A 9(E)-(cyanomethyl)oxime,
4″-O-{2-[3-(6-carboxy-3,3-dimethyl-7-oxo-1H,7H-2-[1,3]oxazino[5,4,3-ij]quinolin-9-yl)propyloxy]ethylcarbamoyl}-erythromycin A 9(E)-methoxymethyloxime,
4″-O-{2-[3-(6-carboxy-3,3-dimethyl-7-oxo-1H,7H-2-[1,3]oxazino[5,4,3-ij]quinolin-9-yl)propyloxy]ethylcarbamoyl}-erythromycin A 9(E)-methyloxime,
4″-O-{2-[3-(6-carboxy-3,3-dimethyl-7-oxo-1H,7H-2-[1,3]oxazino[5,4,3-ij]quinolin-9-yl)propyloxy]ethylcarbamoyl}-6-O-methyl-erythromycin A,
4″-O-{2-[3-(6-carboxy-3,3-dimethyl-7-oxo-1H,7H-2-[1,3]oxazino[5,4,3-ij]quinolin-9-yl)propyloxy]ethylcarbamoyl}-erythromycin A 9(E)-(2-diethylaminoethyl)oxime,
4″-O-{2-[3-(6-carboxy-3,3-dimethyl-7-oxo-1H,7H-2-[1,3]oxazino[5,4,3-ij]quinolin-9-yl)propyloxy]ethylcarbamoyl}-erythromycin A 9(E)-oxime,
4″-O-{2-[3-(6-carboxy-3,3-dimethyl-7-oxo-1H,7H-2-[1,3]oxazino[5,4,3-ij]quinolin-9-yl-propyloxy]-ethylcarbamoyl}-(9S)-9-O-11-O-ethylidene-9-dihydroerythromycin A,
4″-O-{2-[2-(6-carboxy-3,3-dimethyl-7-oxo-1H,7H-2-[1,3]oxazino[5,4,3-ij]quinolin-9-yl)thio)ethoxy]ethylcarbamoyl}-erythromycin A 9(E)-oxime trifluoroacetic acid salt, and
4″-O-{2-[3-(6-carboxy-3,3-dimethyl-7-oxo-1H,7H-2-[1,3]oxazino[5,4,3-ij]quinolin-9-yl)thio)-ethoxy]-ethylcarbamoyl}-erythromycin A 9(E)-methoxymethyloxime trifluoroacetic acid salt, and pharmaceutically acceptable derivatives thereof.

9. A process for the preparation of a compound as claimed in claim 1, or a pharmaceutically acceptable derivative thereof, which comprises reacting a compound of formula (XII), wherein R² is optionally a hydroxyl protecting group, 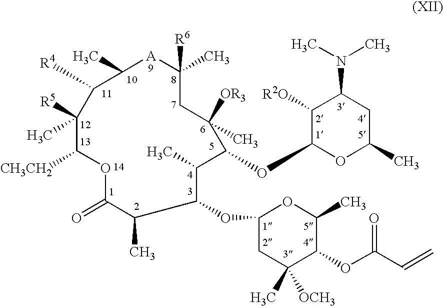
with a compound of formula HU^{1z}R^{14z} (VIII) wherein R^{14z} is R¹⁴ as defined in claim 1 or a group convertible to R¹⁴ and U^{1z} is as defined in claim 1 or a group convertible to U^{1z} in which W is —N(R³⁹)— or —S—, to produce a compound of formula (I) wherein d is 2 and W is
—N(R³⁹)— or —S—,
and thereafter, if required, subjecting the resulting compound to one or more of the following operations:
i) removal of the protecting group R²,
ii) conversion of U^{1z}R^{14z} to U¹R¹⁴, and
iii) conversion of the resultant compound of formula (I) into a pharmaceutically acceptable derivative thereof.

10-12. (canceled)

13. A method for the treatment of the human or non-human animal body to combat microbial infection comprising administration to a body in need of such treatment of an effective amount of a compound as claimed in claim 1, or a pharmaceutically acceptable derivative thereof.

14. A pharmaceutical composition comprising a compound as claimed in claim 1, or a pharmaceutically acceptable derivative thereof, in association with a pharmaceutically acceptable excipient, diluent and/or carrier.
